(19) United States
(12) Patent Application Publication
(10) Pub. No.: US 2024/0423961 A1
(43) Pub. Date: Dec. 26, 2024

(54) 1H-1,2,3-TRIAZOLE-4-CARBOXYLIC ACIDS FOR TREATMENT OF HYPEROXALURIA AND KIDNEY STONES

(71) Applicants: CANTERO THERAPEUTICS, INC., Palo Alto, CA (US); BRIDGEBIO SERVICES INC., Palo Alto, CA (US)

(72) Inventors: Jonathan C. FOX, Palo Alto, CA (US); Uma SINHA, Palo Alto, CA (US); Satish RAO, Palo Alto, CA (US)

(21) Appl. No.: 18/700,172

(22) PCT Filed: Oct. 11, 2022
(86) PCT No.: PCT/US2022/046253

§ 371 Date: Apr. 10, 2024

Related U.S. Application Data
(60) Provisional application No. 63/254,376, filed on Oct. 11, 2021.
(60) Provisional application No. 63/356,002, filed on Jun. 27, 2022.

(51) Int. Cl. A61K 31/4192

(52) U.S. Cl. CPC A61K 31/4192

(57) ABSTRACT

Described herein are methods for treating hyperoxaluria in a subject with Compound 1, represented by the formula: (Compound 1), a tautomer, a pharmaceutically acceptable salt, a hydrate, a solvate, or a combination thereof. In particular, Compound 1 is Compound 1a or a tautomer thereof in a monosodium and monohydrate form. Also described are methods for treating or preventing kidney stones. The methods include specific dosing regimens that have great efficacy in treating the subjects and that are well tolerated in subjects.

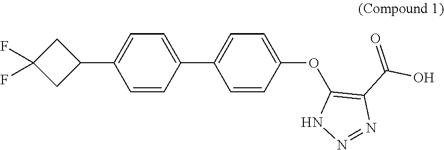 [SMILES: O=C(O)c1nn[nH]c1Oc1ccc(-c2ccc(C3CC(F)(F)C3)cc2)cc1]

### CROSS-REFERENCES TO RELATED APPLICATIONS

This application claims priority to U.S. Provisional Application No. 63/254,376 filed Oct. 11, 2021 and U.S. Provisional Application No. 63/356,002 filed Jun. 27, 2022, each of which is incorporated herein in its entirety for all purposes.

### STATEMENT AS TO RIGHTS TO INVENTIONS MADE UNDER FEDERALLY SPONSORED RESEARCH AND DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK

Not Applicable

### BACKGROUND

Kidney stone disease (KSD) has a prevalence of approximately 10% in developed countries with lifetime recurrence rates of up to 50% [Johri, et al. (2010) Nephron Clin Pract. 116: c159]. KSD patients present with hematuria and renal colic and medical treatment is essentially symptomatic. The administration of drugs to facilitate stone passage is effective for small stones (<5 millimeters (mm)). For bigger stones, extracorporeal sound waves or minimally invasive surgery are used to break the stone into small pieces that can more easily pass the urinary tract [Coe et al. (2005) J. Clin. Invest. 115: 2598].

Approximately 75% of kidney stones contain primarily calcium oxalate and elevated levels of urinary oxalate are found in up to 50% of KSD patients. Furthermore, increased levels of urinary oxalate increase the risk of forming kidney stones [Moe (2006) Lancet 367: 333; Sakhaee (2009) Kidney Int. 75: 585; Kaufman et al. (2008) J Am Soc Nephrol. 19: 1197]. In mammals, calcium has vital physiological roles in many processes, requiring maintenance of blood concentrations within a very narrow range that is achieved through tight regulation of intestinal absorption, uptake or release by bone, and reabsorption or elimination by the kidney. Oxalate is a metabolic end-product with no known physiological role. Oxalate is a divalent anion eliminated in urine and can combine with calcium and precipitate as insoluble calcium oxalate (CaOx) crystals. These crystals can aggregate as stones within the urinary tract as described above or be deposited directly in soft tissues, causing direct damage and impairing organ function.

Hyperoxaluria refers to a metabolic state characterized by an increased urinary excretion of oxalate. Depending on diet, daily urinary oxalate excretion in healthy individuals ranges from 10 milligrams (mg) to 40 mg per 24 hours (0.1-0.45 millimoles per 1.73 square meters per 24 hours (mmol/1.73 m²/24 hr)). The urinary excretion of oxalate at a concentration exceeding 40 to 50 mg per 24 hr (0.45-0.56 mmol/1.73 m²/24 hr) generally defines hyperoxaluria. Supersaturation of urine and other bodily fluids with oxalate can result in the crystallization and accumulation of CaOx crystals. This process most readily occurs in the kidney where it can lead to nephrolithiasis, nephrocalcinosis, renal impairment, and end stage renal disease (ESRD). At very high levels, excess oxalate can lead to systemic oxalosis, in which widespread deposition of CaOx in other organ systems such as the heart, joints, and skin lead to organ dysfunction.

Hyperoxalurias are generally grouped into primary and secondary hyperoxalurias. Secondary hyperoxlaurias are generally caused by increased dietary ingestion of oxalate or its precursors and/or alteration of intestinal microflora. Secondary hyperoxalurias are associated with recurrent kidney stones, nephrocalcinosis, urinary tract infections, chronic kidney disease, and even ESRD. Primary hyperoxalurias (PH) are a group of rare metabolic diseases with autosomal recessive inheritance affecting the glycine/glyoxylate salvage or the hydroxyproline catabolic pathways. To date, three distinct forms of primary hyperoxaluria (primary hyperoxaluria types 1, 2, and 3) have been identified. Primary hyperoxaluria type 1 (PH1) is caused by mutation of liver-specific enzyme alanine-glyoxylate aminotransferase (AGT) and accounts for approximately 80% of all PH patients [Salido et al. (2012) Biochim Biophys Acta. 1822: 1453]. Primary hyperoxaluria type 2 (PH2) is caused by mutation of glyoxylate reductase-hydroxypyruvate reductase (GRHPR). Primary hyperoxaluria type 3 (PH3) is caused by mutation of 4-hydroxy-2-oxoglutarate aldolase (HOGA1). All three forms of PH result have in common an overproduction of oxalate. PH1 displays the most severe clinical phenotype with high penetrance, early age of onset, and rapid progression of renal damage leading to end-stage renal disease (ESRD). PH2 and PH3 display a less severe phenotype and milder clinical course.

Minor increases in urinary oxalate can produce a large effect on calcium oxalate crystal formation, and elevated levels of urinary oxalate are a major risk factor for the formation of calcium oxalate kidney stones [Pak, et al. (2004) Kidney Int. 66: 2032]. Consequently, minor reductions in urinary oxalate concentration could lower combined calcium and oxalate concentrations below saturation (the condition in which the concentrations of calcium and oxalate exceed the solubility of calcium oxalate, favoring spontaneous crystallization and precipitation or aggregation). Lowering urinary calcium and oxalate below saturation could reduce or prevent calcium oxalate stone formation and/or tissue deposition. Irrespective of urinary oxalate levels in individuals with kidney stone disease, lowering urinary oxalate (UOx) levels will decrease the likelihood of calcium oxalate crystallization and potentially lower the probability of stone formation and/or alleviate the severity of excessive calcium oxalate deposition-related conditions [Marengo et al. (2008) Nat Clin Pract Nephrol. 4: 368].

An effective drug that reduces urinary oxalate levels could be a valuable therapeutic option in the prophylaxis and treatment of conditions relating to abnormal levels of oxalate (e.g., calcium oxalate). Common approaches for treatment of urolithiasis due to calcium oxalate include surgical or ureteroscopic removal of stones, extracorporeal lithotripsy, dietary management to increase fluid intake and to restrict oxalate intake, urine alkalization, thiazide diuretics, and crystallization inhibitors such as citrate, bicarbonate, and magnesium [Moe, supra]. However, none of these therapeutic approaches tackles the origin of these conditions. No drug that specifically inhibits the endogenous biosynthetic formation of oxalate is currently available for the prophylaxis and treatment of calcium oxalate deposition-related conditions.

In humans, dietary oxalate contributes 10-50% to excreted urinary oxalate [Holmes, et al. (2001) Kidney Int. 59: 270]. The majority of urinary oxalate is derived from endogenous metabolism, mainly in liver. In humans, the major precursor of oxalate is glyoxylate. Therefore, approaches to reduce the production of oxalate may inhibit the conversion of glyoxylate to oxalate or the production of glyoxylate from its precursors. In humans, glyoxylate is produced predominantly from glycolate in a reaction catalyzed by the peroxisomal liver enzyme glycolate oxidase (GO), also termed hydroxyacid oxidase 1 (HAOX1). Pharmacological inhibition of GO activity is expected to reduce endogenous oxalate production leading to a reduction in calcium oxalate levels in the urine, thus providing a targeted approach for prophylaxis and treatment of calcium oxalate stone formation and tissue deposition-related conditions. Inhibition of GO is hypothesized to be a safe therapeutic target in humans given the observation that an individual harboring a defective splice variant of human GO leading to complete inactivity (spontaneous human null) caused isolated, asymptomatic glycolic aciduria with no apparent ill effects [Frishberg, et al. (2014) J Med Genet. 51: 526]. Kinetic studies have suggested a structural similarity between the substrate binding sites in HAOX1 and lactate dehydrogenase (LDH) (Murray et al, 2008), since both enzymes can oxidize glyoxylate to oxalate (Duncan and Tipton, 1969). Although LDH catalyzes the conversion efficiently, in hyperoxaluria, the formation of oxalate might still be mostly attributed to HAOX1. The impermeability of the peroxisome to glyoxylate makes the substrate inaccessible to LDH, which is present in the cytoplasm. Glyoxylate must be transported out of the peroxisome to be oxidized to oxalate by LDH. High concentration of glyoxylate may have a saturating effect on transporters and the substrate for LDH may remain in the peroxisome (Jones et al, 2000). Thus, GO may be a better target for inhibition of oxalate formation in hyperoxaluria.

Despite ongoing research, there exists a need in the art to provide methods for appropriately dosing pharmacological agents that can treat primary hyperoxaluria, calcium oxalate kidney stones, and calcium oxalate crystal deposition-related ailments. The present disclosure addresses these needs and provides related advantages as well.

### SUMMARY

The present disclosure provides compositions and methods for use in the treatment of hyperoxaluria (e.g., primary hyperoxaluia type I) and frequent or recurrent stone formation. The present disclosure also provides methods of treating one or more symptoms of hyperoxaluria (e.g., primary hyperoxaluria type I) and/or frequent or recurrent stone formation, as well as preventing the development, worsening, or recurrence of kidney stones and/or reducing the size of kidney stones. The methods provided herein comprise administration of a therapeutically effective amount of a compound (e.g., Compound 1, as described herein), or a form thereof, to a subject in need thereof.

In a first aspect, provided herein is a method of treating hyperoxaluria in a subject in need thereof, the method including administering to the subject a therapeutically effective amount of Compound 1, having the formula:

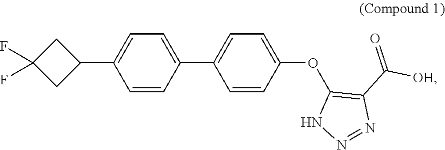 [SMILES: O=C(O)c1nn[nH]c1Oc1ccc(-c2ccc(C3CC(F)(F)C3)cc2)cc1]

a tautomer, a pharmaceutically acceptable salt, a hydrate, a solvate, or a combination thereof, wherein the therapeutically effective amount reduces a maximal urinary oxalate (uOxalate) excretion: i) to less than about 0.46 millimoles per 1.73 square meters per 24 hours (mmol/1.73 m²/24 hr), and/or ii) by at least about 50% relative to a uOxalate excretion prior to the treating.

In a second aspect, provided herein is a method of treating hyperoxaluria in a subject in need thereof, the method including administering to the subject a therapeutically effective amount of Compound 1, having the formula:

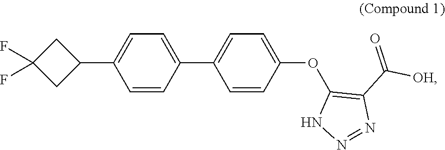 [SMILES: O=C(O)c1nn[nH]c1Oc1ccc(-c2ccc(C3CC(F)(F)C3)cc2)cc1]

a tautomer, a pharmaceutically acceptable salt, a hydrate, a solvate, or a combination thereof, wherein the therapeutically effective amount is a total daily dosage of about 20 milligrams (mg) to about 3,500 mg.

In a third aspect, provided herein is a method of preventing, delaying the onset of, ameliorating, preventing the progression of, alleviating, reducing, improving, eliminating, or curing a symptom and/or complication of hyperoxaluria in a subject in need thereof, the method including administering to the subject a therapeutically effective amount of Compound 1, having the formula:

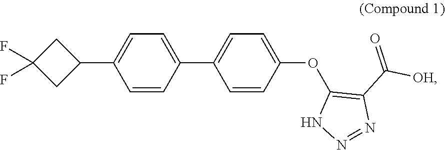 [SMILES: O=C(O)c1nn[nH]c1Oc1ccc(-c2ccc(C3CC(F)(F)C3)cc2)cc1]

a tautomer, a pharmaceutically acceptable salt, a hydrate, a solvate, or a combination thereof, wherein the therapeutically effective amount is a total daily dosage of about 20 milligrams (mg) to about 3,500 mg.

In another aspect, provided herein is a method of lowering a urinary oxalate level in a subject, the method including administering to the subject in need thereof a therapeutically effective amount of Compound 1, having the formula:

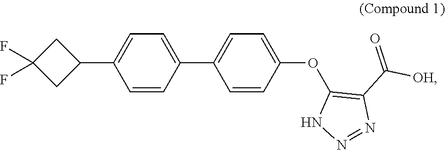 [SMILES: O=C(O)c1nn[nH]c1Oc1ccc(-c2ccc(C3CC(F)(F)C3)cc2)cc1]

a tautomer, a pharmaceutically acceptable salt, a hydrate, a solvate, or a combination thereof, wherein the therapeutically effective amount is a total daily dosage of about 20 milligrams (mg) to about 3,500 mg.

In another aspect, provided herein is a method of lowering a plasma oxalate level in a subject, the method including administering to the subject in need thereof a therapeutically effective amount of Compound 1, having the formula:

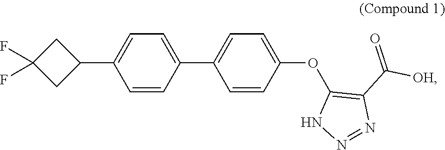 [SMILES: O=C(O)c1nn[nH]c1Oc1ccc(-c2ccc(C3CC(F)(F)C3)cc2)cc1]

a tautomer, a pharmaceutically acceptable salt, a hydrate, a solvate, or a combination thereof, wherein the therapeutically effective amount is a total daily dosage of about 20 milligrams (mg) to about 3,500 mg.

In a further aspect, provided herein is a method of treating kidney stones in a subject in need thereof, the method including administering to the subject a therapeutically effective amount of Compound 1, having the formula:

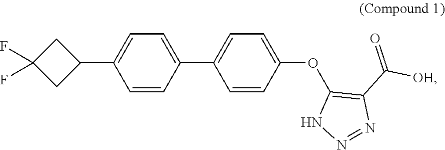 [SMILES: O=C(O)c1nn[nH]c1Oc1ccc(-c2ccc(C3CC(F)(F)C3)cc2)cc1]

a tautomer, a pharmaceutically acceptable salt, a hydrate, a solvate, or a combination thereof, wherein the therapeutically effective amount is a total daily dosage of about 20 mg to about 3,500 mg.

In another aspect, provided herein is a method of preventing the development, worsening, or recurrence of kidney stones in a subject in need thereof, the method including administering to the subject a therapeutically effective amount of Compound 1, having the formula:

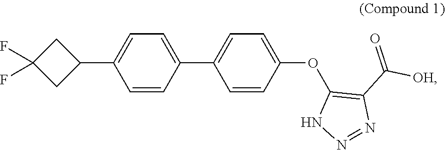 [SMILES: O=C(O)c1nn[nH]c1Oc1ccc(-c2ccc(C3CC(F)(F)C3)cc2)cc1]

or a pharmaceutically acceptable salt, hydrate, solvate, or combination thereof, wherein the therapeutically effective amount is a total daily dosage of about 20 mg to about 3,500 mg.

In another aspect, provided herein is a method of reducing the size of kidney stones in a subject in need thereof, the method comprising administering to the subject a therapeutically effective amount of Compound 1, having the formula:

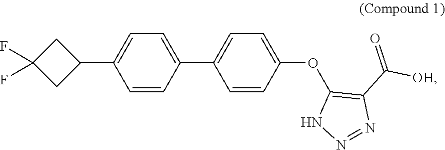 [SMILES: O=C(O)c1nn[nH]c1Oc1ccc(-c2ccc(C3CC(F)(F)C3)cc2)cc1]

a tautomer, a pharmaceutically acceptable salt, a hydrate, a solvate, or a combination thereof, wherein the therapeutically effective amount is a total daily dosage of about 20 mg to about 3,500 mg.

In some embodiments of the preceding aspects, Compound 1 is Compound 1a

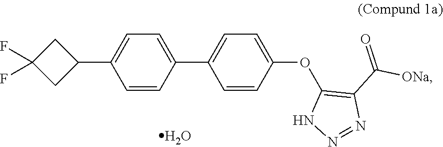

or a tautomer thereof.

Other objects, features, and advantages of the present disclosure will be apparent to one of skill in the art from the following detailed description and figures.

### BRIEF DESCRIPTION OF THE DRAWINGS

FIG. 1 summarizes the details of Phase 1 trial described in Example 1. The trial is a 2-part, randomized, double-blind, placebo-controlled first-in-human study of Compound 1a in healthy adult volunteers. Part A was a single ascending dose (SAD) design consisting of 5 cohorts of 6-8 subjects each randomized in a 3:1 ratio (active:placebo). One cohort participated in 2 sequential dose periods to test for a food effect on the pharmacokinetics of Compound 1a. Part B was a multiple ascending dose (MAD) design consisting of 4 cohorts of 8 subjects each randomized in a 3:1 ratio (active:placebo).

FIGS. 2A-2C show Compound 1a plasma concentration-time profiles following administration of (FIG. 2A) single oral doses of Compound 1a and (FIGS. 2B and 2C) multiple oral doses of Compound 1a every 12 to 24 hours for 7 days. Prior to and following the administration of single oral doses of Compound 1a in the amounts of 40 mg, 120 mg, 360 mg, 1,000 mg, 2,000 mg, and 3,000 mg and multiple oral doses of 75 mg qd, 125 mg q12 h, 500 mg q12 h, 500 mg qd, 750 mg qd, and 1000 mg qd, serial timed blood samples were analyzed for Compound 1a plasma concentration. Values represent arithmetic mean±standard deviation (SD) at each dose.

FIGS. 3A and 3B show plasma glycolate levels after administration of (FIG. 3A) single oral doses of Compound 1a and (FIG. 3B) multiple oral doses of Compound 1a. FIG. 3C shows the mean plasma glycolate concentration in micromolar (M) for multiple dose cohorts. As shown in the figure, Compound 1a inhibited glycolate oxidase (GO) within 2 hours with increased plasma glycolate concentrations. HAO1=hydroxyacid oxidase 1, the gene encoding GO; FDA=US Food and Drug Agency; EMA=European Medicines Agency. Error bars are not shown for visual clarity.

FIG. 4 shows plasma-concentration time profiles of Compound 1a following administration of a single oral dose of Compound 1a in fasted and fed states.

FIGS. 5A and 5B shows Study Schemes of a Phase 2/3 Clinical Study. FIG. 5A schematically illustrates a study scheme for a Phase 2/3 Clinical Study including a dose-finding trial (Part A) and a randomized withdrawal trial (Part B) while FIG. 5B schematically illustrates a study scheme for a Phase 2/3 Clinical Study including a dose-finding trial (Part A) and a parallel group, placebo-controlled trial (Part B), as described in Example 2.

FIGS. 6A and 6B show pharmacokinetic-pharmacodynamic modeling for Phase 2/3 dose selection. FIG. 6A: Predicted plasma Compound 1a concentration (ng/mL); and FIG. 6B: Predicated plasma glycolate concentration (μM).

### DETAILED DESCRIPTION

### I. General

The present disclosure provides methods for treating hyperoxaluria (e.g., primary hyperoxaluria (PH) such as PH type 1, also written as “PH1”) and/or kidney stones in a subject. In some embodiments, the subject has PH1. In some embodiments, the subject has kidney stones (nephrolithiasis) and/or has had kidney stones one or more times. In some embodiments, the subject is at high-risk for developing kidney stones. High risk subjects can include those with a hyperoxaluria (e.g., a primary or secondary hyperoxaluria), as well as those having elevated levels of oxalate (e.g., urinary oxalate), such as those who experience oxalate overproduction that is not directly attributable to germ line genetic variants. The methods include specific dosing regimens that may be generally well tolerated and efficacious for treating the subjects.

Hyperoxaluria is characterized by increased urinary excretion of oxalate (e.g., excretion exceeding 40 milligrams (mg) per 24 hour (hr).

Hyperoxalurias are generally grouped into primary and secondary hyperoxalurias. Secondary hyperoxalurias are generally caused by increased dietary ingestion of oxalate or its precursors and/or alteration of intestinal microflora. Secondary hyperoxalurias are associated with recurrent kidney stones, nephrocalcinosis, urinary tract infections, chronic kidney disease, and even end-stage renal disease (ESRD). In contrast, the primary hyperoxalurias describes a group of genetic disorders characterized by enzymatic abnormalities which lead to an overproduction of oxalate. Primary hyperoxaluria type 1 (PH1), caused by mutations in the alanine-glyoxylate aminotransferase gene (AGXT), is the most severe and prevalent form of primary hyperoxaluria. Mutations in AGXT impair the recycling of glyoxylate to glycine in hepatic peroxisomes and the accumulated glyoxylate is subsequently metabolized to oxalate. The excess oxalate can precipitate as calcium oxalate in the renal tubules leading to nephrocalcinosis, interstitial fibrosis, and chronic kidney disease that can progress to end stage renal disease (ESRD). When the glomerular filtration rate (GFR) drops to 30 to 45 milliliters (mL) per minute per 1.73 square meters (m²) of body surface area, the kidney is unable to effectively excrete the filtered oxalate load and plasma levels rise further, promoting systemic oxalosis. The clinical course of PH1 is variable and based on genotype, age, and extent of disease at diagnosis. Approximately 25% of patients present within the first few months of life, half present in late childhood or early adolescence and the remainder present in adulthood. The disease is relentlessly progressive, and in most patients results in ESRD necessitating transplantation, as dialysis is not particularly effective at removing plasma oxalate.

In individuals without Primary Hyperoxaluria, such as those with secondary hyperoxaluria, elevated excretion of oxalate can be caused by increased dietary absorption of oxalate, endogenous overproduction of oxalate, or a combination of the two. Hyperoxaluria from increased dietary or endogenous production of oxalate also increases the risk for recurrent CaOx nephrolithiasis and is associated with progressive CKD. The prevalence of nephrolithiasis is high (e.g., ˜9% in the United States general population) and has been increasing globally. Clinical stone events cause substantial pain, often require surgical or ureteroscopic intervention, and may be associated with severe complications including infections and renal failure, ultimately resulting in substantial medical resource utilization, reduced job productivity, and adverse effects on lifestyle and quality of life. Presentation typically occurs in working-age adults, resulting in substantial impact to quality of life and functional status. Though increased fluid consumption and thiazide diuretic medications can mildly decrease the risk of recurrent nephrolithiasis, the rate or recurrence following an initial stone event remains high (30% within 10 years), and there are few pharmaceutical options for recurrent stone formers.

### II. Definitions

While various embodiments and aspects of the present disclosure are shown and described herein, it will be obvious to those skilled in the art that such embodiments and aspects are provided by way of example only. Numerous variations, changes, and substitutions will now occur to those skilled in the art without departing from the disclosure. It should be understood that various alternatives to the embodiments of the disclosure described herein may be employed in practicing the disclosure.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY, 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of the invention(s). The following definitions are provided to facilitate understanding of certain terms used frequently herein and are not meant to limit the scope of the present disclosure.

The terms “a” or “an,” as used in herein means one or more.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

“Compound 1” refers to the chemical 5-((4′-(3,3-difluorocyclobutyl)-[1,1′-biphenyl]-4-yl)oxy)-1H-1,2,3-triazole-4-carboxylic acid, having the formula:

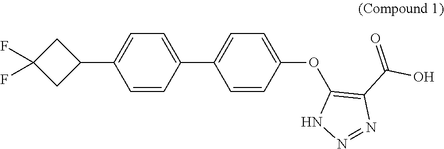 [SMILES: O=C(O)c1nn[nH]c1Oc1ccc(-c2ccc(C3CC(F)(F)C3)cc2)cc1]

a tautomer, a pharmaceutically acceptable salt, hydrate, solvate, or a combination thereof. Compound 1 and related compounds are disclosed in International Patent Application No. PCT/US2019/040690, which is herein incorporated by reference in its entirety.

It will be apparent to one skilled in the art that Compound 1 of this disclosure may exist in tautomeric forms, all such tautomeric forms of Compound 1 being within the scope of the disclosure. Tautomer refers to one of two or more structural isomers which exist in equilibrium and which are readily converted from one isomeric form to another. Generally, this interconversion is sufficiently fast so that an individual tautomer is not isolated in the absence of another tautomer. For example, Compound 1 of the following formulae can exist in equilibrium:

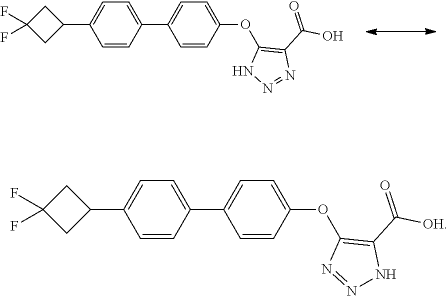 [SMILES: O=C(O)c1nn[nH]c1Oc1ccc(-c2ccc(C3CC(F)(F)C3)cc2)cc1 ; O=C(O)c1[nH]nnc1Oc1ccc(-c2ccc(C3CC(F)(F)C3)cc2)cc1]

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 1 milligram (mg) to 3 mg,” which is intended to include 1 mg, 3 mg, and everything in between to any number of significant figures (e.g., 1.255 mg, 2.1 mg, 2.9999 mg, etc.).

“About” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

“Salt” refers to acid or base salts of the compounds of the present disclosure. Illustrative examples of pharmaceutically acceptable acid addition salts are mineral acid (hydrochloric acid, hydrobromic acid, phosphoric acid, and the like) salts and organic acid (acetic acid, propionic acid, glutamic acid, citric acid and the like) salts. Examples of pharmaceutically acceptable base addition salts include sodium, potassium, calcium, ammonium, organic amino, or magnesium salt, or a similar salt. It is understood that the pharmaceutically acceptable salts are non-toxic. Additional information on suitable pharmaceutically acceptable salts can be found in Remington's Pharmaceutical Sciences, 23rd Edition, 2020, which is incorporated herein by reference.

“Solvate” refers to a compound provided herein or a salt thereof, that further includes a stoichiometric or non-stoichiometric amount of solvent bound by non-covalent intermolecular forces.

“Hydrate” refers to a compound that is complexed to a water molecule. The compounds of the present disclosure can be complexed with 1/2 water molecule or from 1 to 10 water molecules.

“Pharmaceutically acceptable” refers to those compounds (salts, hydrates, solvates, stereoisomers, conformational isomers, tautomers, etc.) which are suitable for use in contact with the tissues of patients without undue toxicity, irritation, and allergic response, are commensurate with a reasonable benefit/risk ratio, and are effective for their intended use. The compounds disclosed herein can exist as pharmaceutically acceptable salts, as defined and described herein.

“Composition,” as used herein, is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present disclosure include, but are not limited to, binders, fillers, glidants, disintegrants, surfactants, lubricants, coatings, sweeteners, flavors, and colors. One of skill in the art will recognize that other pharmaceutical excipients are useful in the present disclosure.

“Tablet” refers to solid pharmaceutical formulations with and without a coating. The term “tablet” also refers to tablets having one, two, three or even more layers, wherein each of the before mentioned types of tablets may be without or with one or more coatings. In some embodiments, tablets of the present disclosure can be prepared by roller compaction or other suitable means known in the art. The term “tablet” also comprises mini, melt, chewable, effervescent, and orally disintegrating tablets. Tablets include Compound 1 or Compound 1a and one or more pharmaceutical excipients (e.g., fillers, binders, glidants, disintegrants, surfactants, binders, lubricants, and the like). Optionally, a coating agent can be also included. For the purposes of calculating percent weight of the tablet formulation, the amount of coating agent is not included in the calculation. That is, percent weights reported herein are of the uncoated tablet unless otherwise specified.

“Administering” refers to therapeutic provision of the compound or a form thereof to a subject, such as by oral administration.

“Treat”, “treatment”, and “treating” refer to an approach for obtaining beneficial or desired results including but not limited to a therapeutic benefit. By therapeutic benefit is meant eradication, elimination, reduction, remission, alleviation, improvement, or amelioration of the underlying disorder being treated, or one or more symptoms or complications thereof. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. Treatment includes causing the clinical symptoms of the disease to slow in development by administration of a composition; suppressing the disease, that is, causing a reduction in the clinical symptoms of the disease; inhibiting the disease, that is, arresting the development of clinical symptoms by administration of a composition after the initial appearance of symptoms; and/or relieving the disease, that is, causing the regression of one or more clinical symptoms by administration of a composition after their initial appearance. For example, certain methods described herein treat kidney stones by decreasing or reducing the occurrence, frequency, or progression of kidney stone formation; or treat kidney stones by decreasing the size of a kidney stone.

An “effective amount,” “therapeutically effective amount,” or a “pharmaceutically effective amount” is an amount sufficient to accomplish a stated purpose (e.g., achieve the effect for which it is administered, treat a disease, reduce enzyme activity, reduce one or more symptoms of a disease or condition). An example of an “effective amount” is an amount sufficient to contribute to the treatment, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). Efficacy can also be expressed as “-fold” increase or decrease. For example, a therapeutically effective amount can have at least a 1.2-fold, 1.5-fold, 2-fold, 5-fold, or more effect over a control. In some examples, an effective amount may be an amount effective to prevent kidney stone development or progression, reduce or decrease kidney stone occurrence or frequency, treat kidney stone disease, treat PH1, or reduce, ameliorate, delay the progression of, delay the onset of, eliminate, or eradicate one or more symptoms of PH1.

“Patient” or “subject” or “subject in need thereof” refers to a living organism suffering from or prone to a disease or condition that can be treated or improved by using the methods provided herein. The term does not necessarily indicate that the subject has been diagnosed with a particular disease, but typically refers to an individual under medical supervision. Non-limiting examples include humans, other mammals, rats, mice, dogs, non-human primates, monkeys, goat, sheep, cows, deer, and other non-mammalian animals. In some embodiments, a patient, subject, or subject in need thereof is human. In some embodiments, a human subject is an adult (e.g., at least 18 years of age). In some embodiments, a human subject is less than 18 years of age, such as between 13-18, 12-18, 10-18, 6-18, 10-13, 6-13, 2-13, 10-12, 6-12, 2-12, 6-10, 2-10, or 2-6 years of age, or any range therein. In some embodiments, a human subject is less than 12 years of age. In some embodiments, a human subject is less than 6 years of age. In some embodiments, a human subject is less than 2 years of age. In some embodiments, a human subject is an infant or neonate. In some embodiments, a human subject is an adolescent of at least 12 years old. In some embodiments, a human subject is an adolescent of at least 12 years old with a body weight of at least 50 kg. In some embodiments, a human subject is a child from 6 to 12 years old. In some embodiments, a human subject is a child from 6 to 12 years old or having a body weight of less than 50 kg.

Unless specifically indicated otherwise, the content of Compound 1a in, e.g., a tablet formulation, is calculated based on the weight of Compound 1a, including the salt and water content. Compound 1 has a molecular weight of 371.34 g/mol and Compound 1a (a monosodium, monohydrate of Compound 1) has a molecular weight of 411.34 g/mol. A tablet containing 75 mg of Compound 1a is equivalent to a table containing 67.7 mg of Compound 1, on a salt-free and anhydrous basis, while a tablet containing 300 mg of Compound 1a is equivalent to a table containing 270.8 mg of Compound 1, on a salt-free and anhydrous basis.

### Iii. Methods

In a first aspect, provided herein is a method of treating hyperoxaluria in a subject in need thereof, the method including administering to the subject a therapeutically effective amount of Compound 1, having the formula:

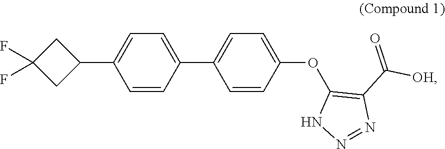 [SMILES: O=C(O)c1nn[nH]c1Oc1ccc(-c2ccc(C3CC(F)(F)C3)cc2)cc1]

a tautomer, a pharmaceutically acceptable salt, a hydrate, a solvate, or a combination thereof, wherein the therapeutically effective amount reduces a maximal urinary oxalate (uOxalate) excretion: i) to less than about 0.46 millimoles per 1.73 square meters per 24 hours (mmol/1.73 m²/24 hr), and/or ii) by at least about 50% relative to a uOxalate excretion prior to the treating.

In some embodiments, administration of the therapeutically effective amount reduces uOxalate excretion: i) to less than about 0.46 millimoles per 1.73 square meters per 24 hours (mmol/1.73 m²/24 hr), or ii) by at least about 50% relative to a uOxalate excretion prior to the treating. In some embodiments, administration of the therapeutically effective amount of Compound 1 reduces uOxalate excretion to less than about 0.46 mmol/1.73 m²/24 hr. In some embodiments, administration of the therapeutically effective amount of Compound 1 reduces uOxalate excretion by at least about 50% relative to a uOxalate excretion prior to the treating. In some embodiments, administration of the therapeutically effective amount reduces uOxalate excretion: i) to less than about 0.46 millimoles per 1.73 square meters per 24 hours (mmol/1.73 m²/24 hr), or ii) by at least about 50% relative to a uOxalate excretion prior to the treating. In some embodiments, administration of the therapeutically effective amount of Compound 1 reduces uOxalate excretion (i) to less than about 0.46 mmol/1.73 m²/24 hr and (ii) by at least about 50% relative to a uOxalate excretion prior to the treating.

In a second aspect, provided herein is a method of treating hyperoxaluria in a subject in need thereof, the method including administering to the subject a therapeutically effective amount of Compound 1, having the formula:

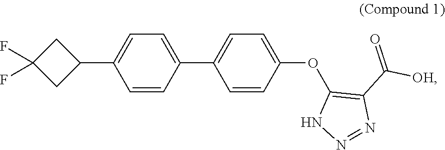 [SMILES: O=C(O)c1nn[nH]c1Oc1ccc(-c2ccc(C3CC(F)(F)C3)cc2)cc1]

a tautomer, a pharmaceutically acceptable salt, a hydrate, a solvate, or a combination thereof, wherein the therapeutically effective amount is a total daily dosage of about 20 milligrams (mg) to about 3,500 mg. In some embodiments, the therapeutically effective amount is a total daily dosage of about 20 milligrams (mg) to about 2,500 mg.

In a third aspect, provided herein is a method of preventing, delaying the onset of, ameliorating, preventing the progression of, alleviating, reducing, improving, eliminating, or curing a symptom and/or complication of hyperoxaluria in a subject in need thereof, the method including administering to the subject a therapeutically effective amount of Compound 1, having the formula:

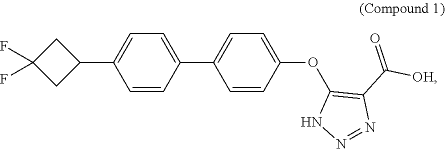 [SMILES: O=C(O)c1nn[nH]c1Oc1ccc(-c2ccc(C3CC(F)(F)C3)cc2)cc1]

a tautomer, a pharmaceutically acceptable salt, a hydrate, a solvate, or a combination thereof, wherein the therapeutically effective amount is a total daily dosage of about 20 milligrams (mg) to about 3,500 mg.

In some embodiments, a symptom and/or complication of hyperoxaluria (e.g., primary hyperoxaluria, such as PH1) is urolithiasis, nephrolithiasis, nephrocalcinosis, recurrent stone formation, chronic kidney disease, kidney failure, end stage renal disease, failure to gain weight, failure to thrive, lethargy, tiredness, loss of appetite, nausea, vomiting, swelling of hands and feet, pale skin color (e.g., relating to anemia), calcium oxalate deposits in kidneys, stones in bladder or urethra, hematuria, dysuria, reduced urine output, urge to urinate often, abdominal pain, renal colic, blockage of urinary tract, repeated urinary tract infections, bedwetting (enuresis), difficulty controlling urine, systemic oxalosis (oxalate accumulation in various organ systems including bone, skin, retinas, myocardium, blood vessels, and/or central nervous system), bone pain, bone fractures, osteosclerosis, abnormal hardening and density of bone, erythropoietin-resistant anemia, optic atrophy, retinopathy, dental pain, tooth mobility, pulp exposure, root resorption, peripheral neuropathy, heart block, arrhythmias, myocarditis, cardioembolic stroke, vasospasm, arthropathy, hepatosplenomegaly, livedo reticularis, necrosis of hands and feet (peripheral gangrene), or calcinosis cutis metastatica.

In another aspect, provided herein is a method of lowering a urinary oxalate level in a subject, the method including administering to the subject in need thereof a therapeutically effective amount of Compound 1, having the formula:

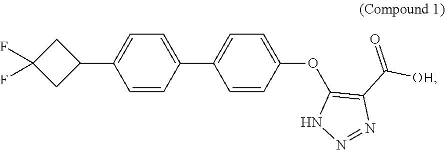 [SMILES: O=C(O)c1nn[nH]c1Oc1ccc(-c2ccc(C3CC(F)(F)C3)cc2)cc1]

a tautomer, a pharmaceutically acceptable salt, a hydrate, a solvate, or a combination thereof, wherein the therapeutically effective amount is a total daily dosage of about 20 milligrams (mg) to about 3,500 mg.

In another aspect, provided herein is a method of lowering a plasma oxalate level in a subject, the method including administering to the subject in need thereof a therapeutically effective amount of Compound 1, having the formula:

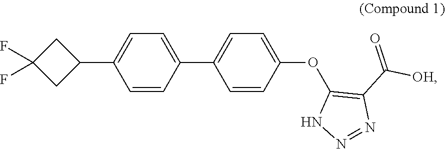 [SMILES: O=C(O)c1nn[nH]c1Oc1ccc(-c2ccc(C3CC(F)(F)C3)cc2)cc1]

a tautomer, a pharmaceutically acceptable salt, a hydrate, a solvate, or a combination thereof, wherein the therapeutically effective amount is a total daily dosage of about 20 milligrams (mg) to about 3,500 mg.

In a further aspect, provided herein is a method of treating kidney stones in a subject in need thereof, the method including administering to the subject a therapeutically effective amount of Compound 1, having the formula:

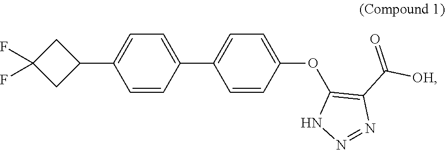 [SMILES: O=C(O)c1nn[nH]c1Oc1ccc(-c2ccc(C3CC(F)(F)C3)cc2)cc1]

a tautomer, a pharmaceutically acceptable salt, a hydrate, a solvate, or a combination thereof, wherein the therapeutically effective amount is a total daily dosage of about 20 mg to about 3,500 mg. In some embodiments, the therapeutically effective amount is a total daily dosage of about 20 mg to about 2,500 mg.

In another aspect, provided herein is a method of preventing the development, worsening, or recurrence of kidney stones in a subject in need thereof, the method including administering to the subject a therapeutically effective amount of Compound 1, having the formula:

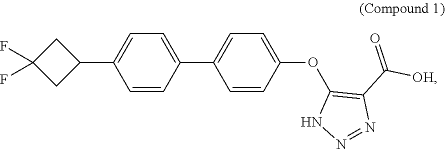 [SMILES: O=C(O)c1nn[nH]c1Oc1ccc(-c2ccc(C3CC(F)(F)C3)cc2)cc1]

or a pharmaceutically acceptable salt, hydrate, solvate, or combination thereof, wherein the therapeutically effective amount is a total daily dosage of about 20 mg to about 3,500 mg. In some embodiments, the therapeutically effective amount is a total daily dosage of about 20 mg to about 2,500 mg.

In another aspect, provided herein is a method of reducing the size of kidney stones in a subject in need thereof, the method comprising administering to the subject a therapeutically effective amount of Compound 1, having the formula:

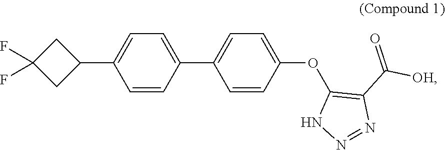 [SMILES: O=C(O)c1nn[nH]c1Oc1ccc(-c2ccc(C3CC(F)(F)C3)cc2)cc1]

a tautomer, a pharmaceutically acceptable salt, a hydrate, a solvate, or a combination thereof, wherein the therapeutically effective amount is a total daily dosage of about 20 mg to about 3,500 mg. In some embodiments, the therapeutically effective amount is a total daily dosage of about 20 mg to about 2,500 mg.

### III-1: Compound 1

Compound 1 or a tautomer thereof can be in a pharmaceutically acceptable salt form or in an acid form (i.e., the acid form as represented by the formula of Compound 1), each of which is optionally in a solvate or a hydrate form.

In some embodiments, Compound 1 is represented by the formula:

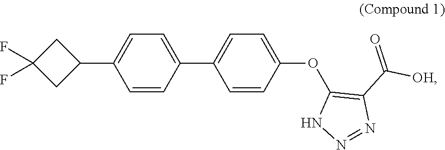 [SMILES: O=C(O)c1nn[nH]c1Oc1ccc(-c2ccc(C3CC(F)(F)C3)cc2)cc1]

or a tautomer thereof, having the name of 5-((4′-(3,3-difluorocyclobutyl)-[1,1′-biphenyl]-4-yl)oxy)-1H-1,2,3-triazole-4-carboxylic acid or 4-((4′-(3,3-difluorocyclobutyl)-[1,1′-biphenyl]-4-yl)oxy)-1H-1,2,3-triazole-5-carboxylic acid.

In some embodiments, Compound 1 or a tautomer thereof is in an acid form represented by the formula. In some embodiments, Compound 1 or a tautomer thereof includes the acid form.

In some embodiments, Compound 1 or a tautomer thereof is in a pharmaceutically acceptable salt form. In some embodiments, a pharmaceutically acceptable base addition salt of Compound 1 is represented by the formula selected from:

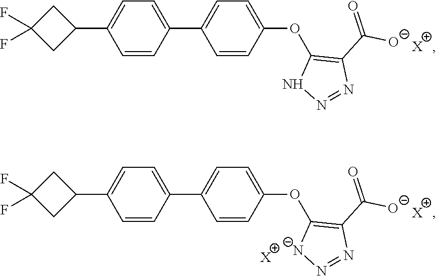

a tautomer thereof, and a combination thereof, wherein each X⁺ is a pharmaceutically acceptable base addition. In some embodiments, Compound 1 is in a mono-salt form represented by the formula:

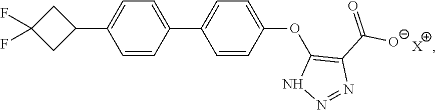

or a tautomer thereof, wherein X⁺ is a pharmaceutically acceptable base addition.

Examples of pharmaceutically acceptable base addition salts include sodium, potassium, calcium, ammonium, organic amino, or magnesium salt, or a similar salt.

In some embodiments, Compound 1 or a tautomer thereof is in a sodium salt form. In some embodiments, Compound 1 or a tautomer thereof is in a disodium salt form, a monosodium salt form, or a combination thereof. In some embodiments, Compound 1 or a tautomer thereof includes a monosodium salt form. In some embodiments, Compound 1 or a tautomer thereof is in a monosodium salt form.

In some embodiments, Compound 1 or a tautomer thereof includes a pharmaceutically acceptable salt thereof and an acid form represented by the formula, each of which is described herein. In some embodiments, Compound 1 or a tautomer thereof includes a monosodium salt form and the acid form.

In some embodiments, Compound 1 or a tautomer thereof is in a hydrate form. In some embodiments, Compound 1 or a tautomer thereof is in a monohydrate form.

In some embodiments, Compound 1 or a tautomer thereof is in a sodium salt, hydrate form. In some embodiments, Compound 1 or a tautomer thereof is in a monosodium salt, monohydrate form.

In some embodiments, Compound 1 is Compound 1a:

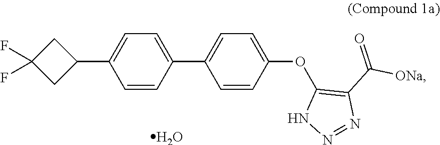

or a tautomer thereof.

In some embodiments, Compound 1a has the name of sodium 5-((4′-(3,3-difluorocyclobutyl)-[1,1′-biphenyl]-4-yl) oxy)-1H-1,2,3-triazole-4-carboxylate monohydrate or sodium 4-((4′-(3,3-difluorocyclobutyl)-[1,1′-biphenyl]-4-yl)oxy)-1H-1,2,3-triazole-5-carboxylate monohydrate.

In some embodiments, Compound 1 or 1a has a purity of at least about 95 area % determined by a high-performance liquid chromatography (HPLC). In some embodiments, Compound 1 or 1a has a purity of from about 95 area % to about 100 area %, from about 96 area % to about 100 area %, from about 97 area % to about 100 area %, from about 98 area % to about 100 area %, or from about 99 area % to about 100 area %, determined by a high-performance liquid chromatography (HPLC).

In some embodiments, Compound 1a contains a disodium salt form in an amount of less than about 10%, about 9%, about 8%, about 7%, about 6%, about 5%, about 4%, about 3%, about 2%, or about 1%. In some embodiments, Compound 1a contains a disodium salt form in an amount of less than about 5%, about 4%, about 3%, about 2%, or about 1%. In some embodiments, Compound 1a contains a disodium salt form in an amount of less than about 5%. In some embodiments, Compound 1a contains a disodium salt form in an amount of less than about 4%. In some embodiments, Compound 1a contains a disodium salt form in an amount of less than about 3%. In some embodiments, Compound 1a contains a disodium salt form in an amount of less than about 2%. In some embodiments, Compound 1a contains a disodium salt form in an amount of less than about 1%. In some embodiments, Compound 1a contains a disodium salt form in an amount of from about 0% to 5%, from about 0% to about 4%, from about 0% to about 3%, from about 0% to about 2%, or from about 0% to about 1%.

In some embodiments, Compound 1a contains an acid form (e.g., represented by the formula of Compound 1) in an amount of less than about 10%, about 9%, about 8%, about 7%, about 6%, about 5%, about 4%, about 3%, about 2%, or about 1%. In some embodiments, Compound 1a contains an acid form in an amount of less than about 5%, about 4%, about 3%, about 2%, or about 1%. In some embodiments, Compound 1a contains an acid form in an amount of less than about 5%. In some embodiments, Compound 1a contains an acid form in an amount of less than about 4%. In some embodiments, Compound 1a contains an acid form in an amount of less than about 3%. In some embodiments, Compound 1a contains an acid form in an amount of less than about 2%. In some embodiments, Compound 1a contains an acid form in an amount of less than about 1%. In some embodiments, Compound 1a contains an acid form in an amount of from about 0% to 5%, from about 0% to about 4%, from about 0% to about 3%, from about 0% to about 2%, or from about 0% to about 1%.

In some embodiments, Compound 1a contains a disodium salt form and an acid form in a combined amount of less than about 10%, about 9%, about 8%, about 7%, about 6%, about 5%, about 4%, about 3%, about 2%, or about 1%. In some embodiments, Compound 1a contains a disodium salt form and an acid form in a combined amount of less than about 5%, about 4%, about 3%, about 2%, or about 1%. In some embodiments, Compound 1a contains a disodium salt form and an acid form in a combined amount of less than about 5%. In some embodiments, Compound 1a contains a disodium salt form and an acid form in a combined amount of less than about 4%. In some embodiments, Compound 1a contains a disodium salt form and an acid form in a combined amount of less than about 3%. In some embodiments, Compound 1a contains a disodium salt form and an acid form in a combined amount of less than about 2%. In some embodiments, Compound 1a contains a disodium salt form and an acid form in a combined amount of less than about 1%. In some embodiments, Compound 1a contains a disodium salt form and an acid form in a combined amount of from about 0% to 5%, from about 0% to about 4%, from about 0% to about 3%, from about 0% to about 2%, or from about 0% to about 1%.

In some embodiments, Compound 1a is substantially free of a disodium salt form and/or an acid form. In some embodiments, a disodium salt form and/or an acid form is absent in Compound 1a. In some embodiments, Compound 1a is substantially free of a disodium salt form. In some embodiments, a disodium salt form is absent in Compound 1a. In some embodiments, Compound 1a is substantially free of an acid form. In some embodiments, an acid form is absent in Compound 1a. In some embodiments, Compound 1a is substantially free of both a disodium salt form and an acid form. In some embodiments, a disodium salt form and an acid form are each absent in Compound 1a.

### III-2: Subject

The subject can have hyperoxaluria and/or kidney stones.

In some embodiments, the subject has hyperoxaluria. In some embodiments, the hyperoxaluria is caused by overproduction of oxalate. In some embodiments, the hyperoxaluria is a primary hyperoxaluria. In some embodiments, the primary hyperoxaluria is primary hyperoxaluria type 1 (PH1). In some embodiments, the subject has primary hyperoxaluria type 1 (PH1).

In some embodiments, the primary hyperoxaluria is primary hyperoxaluria type 2 (PH2) or primary hyperoxaluria type 3 (PH3). In some embodiments, the hyperoxaluria is not caused by a primary hyperoxaluria.

In some embodiments, the subject has primary hyperoxaluria type 1 (PH1) resulting in nephrolithiasis (kidney stones), nephrocalcinosis (deposition of CaOx in the renal parenchyma), and/or progressive chronic kidney disease. In some embodiments, the subject has kidney stones. In some embodiments, the subject has previously had kidney stones. In some embodiments, the subject is at high risk of developing kidney stones.

In some embodiments, the subject has one or more mutations in an alanine-glyoxylate aminotransferase (AGXT) gene. In some embodiments, the subject has primary hyperoxaluria type 1 (PH1) with one or more mutations in an alanine-glyoxylate aminotransferase (AGXT) gene. In some embodiments, the subject has one or more mutations in the GRHPR gene. In some embodiments, the subject has PH type II with one or more mutations in the GRHPR gene. In some embodiments, the subject has one or more mutations in the HOGA1 gene. In some embodiments, the subject has PH type III with one or more mutations in the HOGA1 gene.

In some embodiments, the subject has a maximal urinary oxalate (uOxalate) excretion of more than about 0.7 mmol/1.73 m² per 24 hours. In some embodiments, the subject has a maximal urinary oxalate (uOxalate) excretion of more than about 0.6 mmol/1.73 m² per 24 hours. In some embodiments, the subject has a maximal urinary oxalate (uOxalate) excretion of more than about 0.46 mmol/1.73 m² per 24 hours. For example, the subject may have a uOxalate excretion of more than about 0.47, 0.48, 0.49, 0.50, 0.55, 0.60, 0.65, 0.70, 0.75, or more mmol/1.73 m² per 24 hours.

In some embodiments, the subject has a plasma glycolate (pGlycolate) concentration of greater than about 100 μmol/L. In some embodiments, the subject has a plasma glycolate (pGlycolate) concentration of less than about 100 μmol/L. In some embodiments, the subject has a plasma glycolate (pGlycolate) concentration of at least about 10 μmol/L, about 20 μmol/L, about 30 μmol/L, about 40 μmol/L, about 50 μmol/L, about 60 μmol/L, about 70 μmol/L, about 80 μmol/L, about 90 μmol/L, about 100 μmol/L, or greater. In some embodiments, the subject has a plasma glycolate (pGlycolate) concentration of less than about 100 μmol/L, about 90 μmol/L, about 80 μmol/L, about 70 μmol/L, about 60 μmol/L, about 50 μmol/L, about 40 μmol/L, about 30 μmol/L, about 20 μmol/L, or about 10 μmol/L. In some embodiments, the subject has a plasma glycolate (pGlycolate) concentration of less than about 20 μmol/L. In some embodiments, the subject has a plasma glycolate (pGlycolate) concentration of less than about 15 μmol/L. In some embodiments, the subject has a plasma glycolate (pGlycolate) concentration of less than about 10 μmol/L. For example, the subject may have a pGlycolate concentration of less than about 20, 19, 18, 17, 16, 15, 14, 13, 12, 10, 9, 8, 7, 6, 5, or less μmol/L.

In some embodiments, the subject has an estimated glomerular filtration rate (eGFR) of at least about 10 mL/min/1.73 m². In some embodiments, the subject has an estimated glomerular filtration rate (eGFR) of at least about 20 mL/min/1.73 m². In some embodiments, the subject has an estimated glomerular filtration rate (eGFR) of at least about 30 mL/min/1.73 m². In some embodiments, the subject has an estimated glomerular filtration rate (eGFR) of at least about 50 mL/min/1.73 m². In some embodiments, the subject has an estimated glomerular filtration rate (eGFR) of at least about 90 mL/min/1.73 m². In some embodiments, the subject has an estimated glomerular filtration rate (eGFR) of at least about 100 mL/min/1.73 m². For example, the subject may have an eGFR of at least about 30, 35, 40, 45, 50, 55, 60, 65, 70, 75, 80, 85, 90, 95, 100, 105, 110, 115, 120, or more mL/min/1.73 m². The eGFR is estimated using Chronic Kidney Disease Epidemiology Collaboration refit without the race variable equation for a subject, wherein the subject is a human of at least 18 years old and the Bedside Schwartz equation for a subject, wherein the subject is a human of at least 6 years old, but less than 18 years old.

In some embodiments, the subject receives pyridoxine (Vitamin B6) prior to the treating and during the treating. In some embodiments, the subject receives pyridoxine (Vitamin B6) at a stable dose for at least three (3) months prior to the treating and maintains the same dose during the treating. In some embodiments, the subject has a plasma pyridoxine level within a normal range. In some embodiments, the subject has a plasma pyridoxine level within a normal range, prior to the treating. In some embodiments, the subject has a plasma pyridoxine level within a normal range, prior to and during the treating. In some embodiments, the subject receives pyridoxine (Vitamin B6) at a stable dose for at least three (3) months prior to the treating and maintains the same dose during the treating; and the subject has a plasma pyridoxine level within a normal range prior to the treating.

In some embodiments, the subject is other than: 1) a recipient of a liver transplant; 2) who receives a renal replacement therapy selected from the group consisting of hemodialysis, peritoneal dialysis, a continuous renal replacement therapy, or a combination thereof; and/or 3) who is treated with a ribonucleic acid interference (RNAi) agent (e.g., lumasiran) for at least nine (9) months prior to the treating. In some embodiments, the subject is not a recipient of a liver transplant. In some embodiments, the subject does not receive a renal replacement therapy selected from the group consisting of hemodialysis, peritoneal dialysis, a continuous renal replacement therapy, or a combination thereof. In some embodiments, the subject is not treated with a ribonucleic acid interference (RNAi) agent (e.g., lumasiran) for at least nine (9) months prior to the treating. In some embodiments, the RNAi agent is lumasiran.

In some embodiments, the subject has not received another treatment for a hyperoxaluria or kidney stones prior to treatment with Compound 1. In some embodiments, the subject has not received another treatment for PH1 prior to treatment with Compound 1. In some embodiments, the subject has not received another treatment for kidney stones prior to treatment with Compound 1. In some embodiments, the subject has not received another treatment for PH1 or kidney stones for at least one (1) month prior to treatment with Compound 1, such as for at least 1, 2, 3, 4, 5, 6, 7, 8, 9, 10, 11, or 12 months or longer. In some embodiments, the subject has not received any investigational medicinal product within 30 days or 5 half-lives, whichever is longer, prior to the initiation of the treating; or is in follow-up for another interventional clinical study prior to the treating; and/or 5) who is treated with an experimental RNAi agent (e.g., nedosiran) for at least nine (9) months prior to the treating

In some embodiments, the subject meets all of inclusion criteria as described in Example 2. In some embodiments, the subject meets all of inclusion criteria as described in Example 2, provided that the subject does not meet any one of exclusion criteria as described in Example 2.

In some embodiments, the subject is a mammal. In some embodiments, the subject is human. In some embodiments, a human subject is an adult (e.g., at least 18 years of age). In some embodiments, a human subject is less than 18 years of age, such as between 13-18, 12-18, 10-18, 6-18, 10-13, 6-13, 2-13, 10-12, 6-12, 2-12, 6-10, 2-10, or 2-6 years of age, or any range therein. In some embodiments, a human subject is less than 12 years of age. In some embodiments, a human subject is less than 6 years of age. In some embodiments, a human subject is less than 2 years of age. In some embodiments, a human subject is an infant or neonate. In some embodiments, a human subject is an adolescent of at least 12 years old. In some embodiments, a human subject is an adolescent of at least 12 years old with a body weight of at least 50 kg. In some embodiments, a human subject is a child from 6 to 12 years old. In some embodiments, a human subject is a child from 6 to 12 years old or having a body weight of less than 50 kg.

### III-3: Treatment Period

Treatment with Compound 1 or 1a can include Part A treatment (e.g., various groups with a fixed daily dosage of Compound 1 or 1a), Part B treatment (e.g., with a daily dosage selected and including an optional withdrawal period), and/or a long-term extension (LTE) treatment (e.g., with a daily dosage of Compound 1 or 1a of Part B). In some embodiments, treatment with Compound 1 or 1a is chronic (e.g., lasting at least 1 month, 3 months, 6 months, 9 months, 12 months, 18 months, 2 years, 3 years, or longer, such as the duration of the subject's life).

With reference to Part A treatment, in some embodiments, the treatment includes a single treatment period of Part A (e.g., 28 days), optionally an extension period of Part A (e.g., up to 12 months). In some embodiments, the treatment includes a single treatment period of Part A (e.g., 28 days) and an extension period of Part A (e.g., up to 12 months) after the single treatment period. In some embodiments, the treatment includes an extension period of Part A (e.g., up to 12 months).

In some embodiments, the treatment includes a single treatment period of Part A (e.g., 28 days), optionally an extension period of Part A (e.g., up to 12 months), while a daily dosage of Compound 1 or 1a is maintained. In some embodiments, the treatment includes a single treatment period of Part A (e.g., 28 days) and an extension period of Part A (e.g., up to 12 months) after the single treatment period, while a daily dosage of Compound 1 or 1a is maintained.

In some embodiments, the single treatment period of Part A has a duration of about 28 days. In some embodiments, the extension period of Part A has a duration of no more than 12 months. In some embodiments, the extension period of Part A has a duration of from 1 to 12 months.

With reference to Part B treatment, in some embodiments, the treatment includes a single treatment period of Part B (e.g., 28 days or 6 months), an optional withdrawal period of Part B (e.g., 28 days), and a long-term extension period of Part B (e.g., up to 24 months), wherein, during the optional withdrawal period, no compound 1 or 1a is administered. In some embodiments, the treatment includes a single treatment period of Part B (e.g., 28 days or 6 months), an optional withdrawal period of Part B (e.g., 28 days) after the single treatment period, and a long-term extension period of Part B (e.g., up to 24 months) after the optional withdrawal period.

In some embodiments, during Part B treatment, Compound 1 or 1a is administered at a daily dosage determined by Part A. In some embodiments, during the single treatment and the extension periods of Part B, Compound 1 or 1a is administered at a daily dosage determined by Part A treatment. In some embodiments, during the single treatment and the extension periods of Part B, a daily dosage of Compound 1 or 1a is maintained. In some embodiments, during the single treatment and the extension periods of Part B, Compound 1 or 1a is administered at a daily dosage determined by Part A and the daily dosage of Compound 1 or 1a is maintained.

In some embodiments, the single treatment period of Part B has a duration of about 28 days. In some embodiments, the single treatment period of Part B has a duration of about 6 months. In some embodiments, the optional withdrawal period of Part B has a duration of about 28 days. In some embodiments, the long-term extension period of Part B has a duration of about 28 days. In some embodiments, the long-term extension period of Part B has a duration of up to 24 months.

With reference to the long-term extension (LTE) treatment (e.g., up to 36 months or up to 24 months), in some embodiments, Compound 1 or 1a is administered at a daily dosage determined by Part B. In some embodiments, Compound 1 or 1a is administered at a daily dosage determined by Part B for a duration of from about 1 to about 36 months, from about 1 to about 24 months, from about 6 to about 36 months, from about 6 to about 24 months, from about 12 to about 36 months, from about 12 to about 24 months, or from about 24 to about 36 months. In some embodiments, Compound 1 or 1a is administered at a daily dosage determined by Part B for a duration of about 36 months. In some embodiments, Compound 1 or 1a is administered at a daily dosage determined by Part B for a duration of about 24 months.

With reference to each of Part A, Part B, and LTE treatments, except for the withdrawal period of Part B, Compound 1 or 1a is administered daily (e.g., once, twice, three times, or four times daily). In some embodiments, during Part A treatment, Compound 1 or 1a is administered daily (e.g., once, twice, three times, or four times daily). In some embodiments, during the single treatment and the extension periods of Part B, Compound 1 or 1a is administered daily (e.g., once, twice, three times, or four times daily). In some embodiments, during the LTE treatment, Compound 1 or 1a is administered daily (e.g., once, twice, three times, or four times daily).

In some embodiments, the subject is treated with Compound 1 or 1a for at least 1 month, 3 months, 6 months, 9 months, 12 months, 18 months, 2 years, 3 years, or longer, such as the duration of the subject's life. In some embodiments, treatment of the subject is continuous (e.g., without a withdrawal or holiday period). In some embodiments, treatment of the subject comprises one or more withdrawal or holiday periods (e.g., periods in which Compound 1 or 1a is not administered to the subject).

### III-4: Therapeutically Effective Amount/Administration

In some embodiments, the amount of Compound 1 listed in this application is the amount of Na salt, monohydrate of Compound 1 (Compound 1a) administered. A person of skill in the art would recognize that in order to administer the same amount of Compound 1 in an acid form or in a different salt form, small adjustments in an overall amount administered is necessary. For example, a daily dosage of about 75 mg, 300 mg, or 600 mg of Compound 1a equals to a daily dosage of about 67.7 mg, 270.8 mg, or 541.6 mg of Compound 1 on a salt-free and anhydrous basis, respectively.

Accordingly, a tablet containing 75 mg of Compound 1a is equivalent to a table containing 67.7 mg of Compound 1, on a salt-free and anhydrous basis, while a tablet containing 300 mg of Compound 1a is equivalent to a table containing 270.8 mg of Compound 1, on a salt-free and anhydrous basis.

In some embodiments, the therapeutically effective amount is a total daily dosage of from about 5 mg to about 5,000 mg of Compound 1, a tautomer, a pharmaceutically acceptable salt, a hydrate, a solvate, or a combination thereof. In some embodiments, the therapeutically effective amount is a total daily dosage of from about 5 mg to about 2,500 mg of Compound 1, a tautomer, a pharmaceutically acceptable salt, a hydrate, a solvate, or a combination thereof. In some embodiments, the therapeutically effective amount is a total daily dosage of from about 5 mg to about 5,000 mg, from about 5 mg to about 4,500 mg, from about 5 mg to about 4,000 mg, from about 5 mg to about 3,500 mg, from about 5 mg to about 3,000 mg, from about 5 mg to about 2,500 mg, from about 5 mg to about 2,000 mg, from about 5 mg to about 1,500 mg, from about 5 mg to about 1,400 mg, from about 5 mg to about 1,300 mg, from about 5 mg to about 1,200 mg, from about 5 mg to about 1,100 mg, from about 5 mg to about 1,000 mg, from about 5 mg to about 900 mg, from about 5 mg to about 800 mg, from about 5 mg to about 700 mg, from about 5 mg to about 600 mg, from about 5 mg to about 550 mg, from about 5 mg to about 500 mg, from about 5 mg to about 450 mg, from about 5 mg to about 400 mg, from about 5 mg to about 350 mg, from about 5 mg to about 300 mg, from about 5 mg to about 250 mg, from about 5 mg to about 200 mg, from about 5 mg to about 150 mg, from about 5 mg to about 100 mg, 20 mg to about 5,000 mg, from about 20 mg to about 4,500 mg, from about 20 mg to about 4,000 mg, from about 20 mg to about 3,500 mg, from about 20 mg to about 3,000 mg, from about 20 mg to about 2,500 mg, from about 20 mg to about 2,000 mg, from about 20 mg to about 1,500 mg, from about 20 mg to about 1,400 mg, from about 20 mg to about 1,300 mg, from about 20 mg to about 1,200 mg, from about 20 mg to about 1,100 mg, from about 20 mg to about 1,000 mg, from about 20 mg to about 900 mg, from about 20 mg to about 800 mg, from about 20 mg to about 700 mg, from about 20 mg to about 600 mg, from about 20 mg to about 550 mg, from about 20 mg to about 500 mg, from about 20 mg to about 450 mg, from about 20 mg to about 400 mg, from about 20 mg to about 350 mg, from about 20 mg to about 300 mg, from about 20 mg to about 250 mg, from about 20 mg to about 200 mg, from about 20 mg to about 150 mg, from about 20 mg to about 100 mg, 50 mg to about 5,000 mg, from about 50 mg to about 4,500 mg, from about 50 mg to about 4,000 mg, from about 50 mg to about 3,500 mg, from about 50 mg to about 3,000 mg, from about 50 mg to about 2,500 mg, from about 50 mg to about 2,000 mg, from about 50 mg to about 1,500 mg, from about 50 mg to about 1,400 mg, from about 50 mg to about 1,300 mg, from about 50 mg to about 1,200 mg, from about 50 mg to about 1,100 mg, from about 50 mg to about 1,000 mg, from about 50 mg to about 900 mg, from about 50 mg to about 800 mg, from about 50 mg to about 700 mg, from about 50 mg to about 600 mg, from about 50 mg to about 550 mg, from about 50 mg to about 500 mg, from about 50 mg to about 450 mg, from about 50 mg to about 400 mg, from about 50 mg to about 350 mg, from about 50 mg to about 300 mg, from about 50 mg to about 250 mg, from about 50 mg to about 200 mg, from about 50 mg to about 150 mg, from about 50 mg to about 100 mg, from about 75 mg to about 5,000 mg, from about 75 mg to about 4,500 mg, from about 75 mg to about 4,000 mg, from about 75 mg to about 3,500 mg, from about 75 mg to about 3,000 mg, from about 75 mg to about 2,500 mg, from about 75 mg to about 2,000 mg, from about 75 mg to about 1,500 mg, from about 75 mg to about 1,400 mg, from about 75 mg to about 1,300 mg, from about 75 mg to about 1,200 mg, from about 75 mg to about 1,100 mg, from about 75 mg to about 1,000 mg, from about 75 mg to about 900 mg, from about 75 mg to about 800 mg, from about 75 mg to about 700 mg, from about 75 mg to about 600 mg, from about 75 mg to about 550 mg, from about 75 mg to about 500 mg, from about 75 mg to about 450 mg, from about 75 mg to about 400 mg, from about 75 mg to about 350 mg, from about 75 mg to about 300 mg, from about 75 mg to about 250 mg, from about 75 mg to about 200 mg, from about 75 mg to about 150 mg, from about 75 mg to about 100 mg, from about 300 mg to about 5,000 mg, from about 300 mg to about 4,500 mg, from about 300 mg to about 4,000 mg, from about 300 mg to about 3,500 mg, from about 300 mg to about 3,000 mg, from about 300 mg to about 2,500 mg, from about 300 mg to about 2,000 mg, from about 300 mg to about 1,500 mg, from about 300 mg to about 1,400 mg, from about 300 mg to about 1,300 mg, from about 300 mg to about 1,200 mg, from about 300 mg to about 1,100 mg, from about 300 mg to about 1,000 mg, from about 300 mg to about 900 mg, from about 300 mg to about 800 mg, from about 300 mg to about 700 mg, from about 300 mg to about 600 mg, from about 300 mg to about 550 mg, from about 300 mg to about 500 mg, from about 300 mg to about 450 mg, from about 300 mg to about 400 mg, or from about 300 mg to about 350 mg of Compound 1, a tautomer, a pharmaceutically acceptable salt, a hydrate, a solvate, or a combination thereof.

In some embodiments, the therapeutically effective amount is a total daily dosage of about 75 mg to about 600 mg of Compound 1, a tautomer, a pharmaceutically acceptable salt, a hydrate, a solvate, or a combination thereof. In some embodiments, the therapeutically effective amount is a total daily dosage of about 75 mg to about 300 mg of Compound 1, a tautomer, a pharmaceutically acceptable salt, a hydrate, a solvate, or a combination thereof. In some embodiments, the therapeutically effective amount is a total daily dosage of about 300 mg to about 600 mg of Compound 1, a tautomer, a pharmaceutically acceptable salt, a hydrate, a solvate, or a combination thereof. In some embodiments, the therapeutically effective amount is a total daily dosage of about 5 mg to about 600 mg of Compound 1, a tautomer, a pharmaceutically acceptable salt, a hydrate, a solvate, or a combination thereof. In some embodiments, the therapeutically effective amount is a total daily dosage of about 5 mg to about 300 mg of Compound 1, a tautomer, a pharmaceutically acceptable salt, a hydrate, a solvate, or a combination thereof. In some embodiments, the therapeutically effective amount is a total daily dosage of about 5 mg to about 200 mg of Compound 1, a tautomer, a pharmaceutically acceptable salt, a hydrate, a solvate, or a combination thereof. In some embodiments, the therapeutically effective amount is a total daily dosage of about 5 mg to about 150 mg of Compound 1, a tautomer, a pharmaceutically acceptable salt, a hydrate, a solvate, or a combination thereof. In some embodiments, the therapeutically effective amount is a total daily dosage of about 5 mg to about 100 mg of Compound 1, a tautomer, a pharmaceutically acceptable salt, a hydrate, a solvate, or a combination thereof.

In some embodiments, the total daily dosage of Compound 1, a tautomer, a pharmaceutically acceptable salt, a hydrate, a solvate, or a combination thereof is about 30 mg to about 60 mg. In some embodiments, the total daily dosage of Compound 1, a tautomer, a pharmaceutically acceptable salt, a hydrate, a solvate, or a combination thereof is about 60 mg to about 90 mg. In some embodiments, the total daily dosage of Compound 1, a tautomer, a pharmaceutically acceptable salt, a hydrate, a solvate, or a combination thereof is about 90 mg to about 120 mg. In some embodiments, the total daily dosage of Compound 1, a tautomer, a pharmaceutically acceptable salt, a hydrate, a solvate, or a combination thereof is about 100 mg to about 140 mg. In some embodiments, the total daily dosage of Compound 1, a tautomer, a pharmaceutically acceptable salt, a hydrate, a solvate, or a combination thereof is about 120 mg to about 150 mg. In some embodiments, the total daily dosage of Compound 1, a tautomer, a pharmaceutically acceptable salt, a hydrate, a solvate, or a combination thereof is about 150 mg to about 180 mg. In some embodiments, the total daily dosage of Compound 1, a tautomer, a pharmaceutically acceptable salt, a hydrate, a solvate, or a combination thereof is about 180 mg to about 210 mg. In some embodiments, the total daily dosage of Compound 1, a tautomer, a pharmaceutically acceptable salt, a hydrate, a solvate, or a combination thereof is about 210 mg to about 240 mg. In some embodiments, the total daily dosage of Compound 1, a tautomer, a pharmaceutically acceptable salt, a hydrate, a solvate, or a combination thereof is about 240 mg to about 270 mg. In some embodiments, the total daily dosage of Compound 1, a tautomer, a pharmaceutically acceptable salt, a hydrate, a solvate, or a combination thereof is about 270 mg to about 300 mg. In some embodiments, the total daily dosage of Compound 1, a tautomer, a pharmaceutically acceptable salt, a hydrate, a solvate, or a combination thereof is about 300 mg to about 330 mg. In some embodiments, the total daily dosage of Compound 1, a tautomer, a pharmaceutically acceptable salt, a hydrate, a solvate, or a combination thereof is about 330 mg to about 360 mg. In some embodiments, the total daily dosage of Compound 1, a tautomer, a pharmaceutically acceptable salt, a hydrate, a solvate, or a combination thereof is about 360 mg to about 390 mg. In some embodiments, the total daily dosage of Compound 1, a tautomer, a pharmaceutically acceptable salt, a hydrate, a solvate, or a combination thereof is about 390 mg to about 420 mg. In some embodiments, the total daily dosage of Compound 1, a tautomer, a pharmaceutically acceptable salt, a hydrate, a solvate, or a combination thereof is about 420 mg to about 450 mg. In some embodiments, the total daily dosage of Compound 1, a tautomer, a pharmaceutically acceptable salt, a hydrate, a solvate, or a combination thereof is about 450 mg to about 480 mg. In some embodiments, the total daily dosage of Compound 1, a tautomer, a pharmaceutically acceptable salt, a hydrate, a solvate, or a combination thereof is about 480 mg to about 510 mg. In some embodiments, the total daily dosage of Compound 1, a tautomer, a pharmaceutically acceptable salt, a hydrate, a solvate, or a combination thereof is about 510 mg to about 540 mg. In some embodiments, the total daily dosage of Compound 1, a tautomer, a pharmaceutically acceptable salt, a hydrate, a solvate, or a combination thereof is about 540 mg to about 570 mg. In some embodiments, the total daily dosage of Compound 1, a tautomer, a pharmaceutically acceptable salt, a hydrate, a solvate, or a combination thereof is about 570 mg to about 600 mg. In some embodiments, the total daily dosage of Compound 1, a tautomer, a pharmaceutically acceptable salt, a hydrate, a solvate, or a combination thereof is about 250 mg to about 500 mg. In some embodiments, the total daily dosage of Compound 1, a tautomer, a pharmaceutically acceptable salt, a hydrate, a solvate, or a combination thereof is about 300 mg to about 400 mg. In some embodiments, the total daily dosage of Compound 1, a tautomer, a pharmaceutically acceptable salt, a hydrate, a solvate, or a combination thereof is about 330 mg to about 370 mg. In some embodiments, the total daily dosage of Compound 1, a tautomer, a pharmaceutically acceptable salt, a hydrate, a solvate, or a combination thereof is about 450 mg to about 550 mg. In some embodiments, the total daily dosage of Compound 1, a tautomer, a pharmaceutically acceptable salt, a hydrate, a solvate, or a combination thereof is about 500 mg to about 700 mg. In some embodiments, the total daily dosage of Compound 1, a tautomer, a pharmaceutically acceptable salt, a hydrate, a solvate, or a combination thereof is about 600 mg to about 800 mg. In some embodiments, the total daily dosage of Compound 1, a tautomer, a pharmaceutically acceptable salt, a hydrate, a solvate, or a combination thereof is about 700 mg to about 800 mg. In some embodiments, the total daily dosage of Compound 1, a tautomer, a pharmaceutically acceptable salt, a hydrate, a solvate, or a combination thereof is about 800 mg to about 1000 mg. In some embodiments, the total daily dosage of Compound 1, a tautomer, a pharmaceutically acceptable salt, a hydrate, a solvate, or a combination thereof is about 970 mg to about 1030 mg. In some embodiments, the total daily dosage of Compound 1, a tautomer, a pharmaceutically acceptable salt, a hydrate, a solvate, or a combination thereof is about 1000 mg to about 1500 mg. In some embodiments, the total daily dosage of Compound 1, a tautomer, a pharmaceutically acceptable salt, a hydrate, a solvate, or a combination thereof is about 1500 mg to about 2000 mg. In some embodiments, the total daily dosage of Compound 1, a tautomer, a pharmaceutically acceptable salt, a hydrate, a solvate, or a combination thereof is about 2000 mg to about 2500 mg. In some embodiments, the total daily dosage of Compound 1, a tautomer, a pharmaceutically acceptable salt, a hydrate, a solvate, or a combination thereof is about 1960 mg to about 2040 mg. In some embodiments, the total daily dosage of Compound 1, a tautomer, a pharmaceutically acceptable salt, a hydrate, a solvate, or a combination thereof is about 2500 mg to about 3000 mg. In some embodiments, the total daily dosage of Compound 1, a tautomer, a pharmaceutically acceptable salt, a hydrate, a solvate, or a combination thereof is about 3000 mg to about 3500 mg. In some embodiments, the total daily dosage of Compound 1, a tautomer, a pharmaceutically acceptable salt, a hydrate, a solvate, or a combination thereof is about 2060 mg to about 3040 mg.

In some embodiments, the total daily dosage of Compound 1, a tautomer, a pharmaceutically acceptable salt, a hydrate, a solvate, or a combination thereof is about 230 mg to about 270 mg. In some embodiments, the total daily dosage of Compound 1, a tautomer, a pharmaceutically acceptable salt, a hydrate, a solvate, or a combination thereof is about 470 mg to about 530 mg. In some embodiments, the total daily dosage of Compound 1, a tautomer, a pharmaceutically acceptable salt, a hydrate, a solvate, or a combination thereof is about 730 mg to about 780 mg.

In some embodiments, the total daily dosage of Compound 1, a tautomer, a pharmaceutically acceptable salt, a hydrate, a solvate, or a combination thereof is about 20 mg. In some embodiments, the total daily dosage of Compound 1, a tautomer, a pharmaceutically acceptable salt, a hydrate, a solvate, or a combination thereof is about 40 mg. In some embodiments, the total daily dosage of Compound 1, a tautomer, a pharmaceutically acceptable salt, a hydrate, a solvate, or a combination thereof is about 75 mg. In some embodiments, the total daily dosage of Compound 1, a tautomer, a pharmaceutically acceptable salt, a hydrate, a solvate, or a combination thereof is about 120 mg. In some embodiments, the total daily dosage of Compound 1, a tautomer, a pharmaceutically acceptable salt, a hydrate, a solvate, or a combination thereof is about 125 mg. In some embodiments, the total daily dosage of Compound 1, a tautomer, a pharmaceutically acceptable salt, a hydrate, a solvate, or a combination thereof is about 300 mg. In some embodiments, the total daily dosage of Compound 1, a tautomer, a pharmaceutically acceptable salt, a hydrate, a solvate, or a combination thereof is about 360 mg. In some embodiments, the total daily dosage of Compound 1, a tautomer, a pharmaceutically acceptable salt, a hydrate, a solvate, or a combination thereof is about 1000 mg. In some embodiments, the total daily dosage of Compound 1, a tautomer, a pharmaceutically acceptable salt, a hydrate, a solvate, or a combination thereof is about 2000 mg. In some embodiments, the total daily dosage of Compound 1, a tautomer, a pharmaceutically acceptable salt, a hydrate, a solvate, or a combination thereof is about 250 mg. In some embodiments, the total daily dosage of Compound 1, a tautomer, a pharmaceutically acceptable salt, a hydrate, a solvate, or a combination thereof is about 500 mg. In some embodiments, the total daily dosage of Compound 1, a tautomer, a pharmaceutically acceptable salt, a hydrate, a solvate, or a combination thereof is about 600 mg. In some embodiments, the total daily dosage of Compound 1, a tautomer, a pharmaceutically acceptable salt, a hydrate, a solvate, or a combination thereof is about 750 mg.

In some embodiments, the therapeutically effective amount is a total daily dosage of from about 5 mg to about 5,000 mg, from about 5 mg to about 4,500 mg, from about 5 mg to about 4,000 mg, from about 5 mg to about 3,500 mg, from about 5 mg to about 3,000 mg, from about 5 mg to about 2,500 mg, from about 5 mg to about 2,000 mg, from about 5 mg to about 1,500 mg, from about 5 mg to about 1,400 mg, from about 5 mg to about 1,300 mg, from about 5 mg to about 1,200 mg, from about 5 mg to about 1,100 mg, from about 5 mg to about 1,000 mg, from about 5 mg to about 900 mg, from about 5 mg to about 800 mg, from about 5 mg to about 700 mg, from about 5 mg to about 600 mg, from about 5 mg to about 550 mg, from about 5 mg to about 500 mg, from about 5 mg to about 450 mg, from about 5 mg to about 400 mg, from about 5 mg to about 350 mg, from about 5 mg to about 300 mg, from about 5 mg to about 250 mg, from about 5 mg to about 200 mg, from about 5 mg to about 150 mg, from about 5 mg to about 100 mg, from about 20 mg to about 5,000 mg, from about 20 mg to about 4,500 mg, from about 20 mg to about 4,000 mg, from about 20 mg to about 3,500 mg, from about 20 mg to about 3,000 mg, from about 20 mg to about 2,500 mg, from about 20 mg to about 2,000 mg, from about 20 mg to about 1,500 mg, from about 20 mg to about 1,400 mg, from about 20 mg to about 1,300 mg, from about 20 mg to about 1,200 mg, from about 20 mg to about 1,100 mg, from about 20 mg to about 1,000 mg, from about 20 mg to about 900 mg, from about 20 mg to about 800 mg, from about 20 mg to about 700 mg, from about 20 mg to about 600 mg, from about 20 mg to about 550 mg, from about 20 mg to about 500 mg, from about 20 mg to about 450 mg, from about 20 mg to about 400 mg, from about 20 mg to about 350 mg, from about 20 mg to about 300 mg, from about 20 mg to about 250 mg, from about 20 mg to about 200 mg, from about 20 mg to about 150 mg, from about 20 mg to about 100 mg, 50 mg to about 5,000 mg, from about 50 mg to about 4,500 mg, from about 50 mg to about 4,000 mg, from about 50 mg to about 3,500 mg, from about 50 mg to about 3,000 mg, from about 50 mg to about 2,500 mg, from about 50 mg to about 2,000 mg, from about 50 mg to about 1,500 mg, from about 50 mg to about 1,400 mg, from about 50 mg to about 1,300 mg, from about 50 mg to about 1,200 mg, from about 50 mg to about 1,100 mg, from about 50 mg to about 1,000 mg, from about 50 mg to about 900 mg, from about 50 mg to about 800 mg, from about 50 mg to about 700 mg, from about 50 mg to about 600 mg, from about 50 mg to about 550 mg, from about 50 mg to about 500 mg, from about 50 mg to about 450 mg, from about 50 mg to about 400 mg, from about 50 mg to about 350 mg, from about 50 mg to about 300 mg, from about 50 mg to about 250 mg, from about 50 mg to about 200 mg, from about 50 mg to about 150 mg, from about 50 mg to about 100 mg, from about 75 mg to about 5,000 mg, from about 75 mg to about 4,500 mg, from about 75 mg to about 4,000 mg, from about 75 mg to about 3,500 mg, from about 75 mg to about 3,000 mg, from about 75 mg to about 2,500 mg, from about 75 mg to about 2,000 mg, from about 75 mg to about 1,500 mg, from about 75 mg to about 1,400 mg, from about 75 mg to about 1,300 mg, from about 75 mg to about 1,200 mg, from about 75 mg to about 1,100 mg, from about 75 mg to about 1,000 mg, from about 75 mg to about 900 mg, from about 75 mg to about 800 mg, from about 75 mg to about 700 mg, from about 75 mg to about 600 mg, from about 75 mg to about 550 mg, from about 75 mg to about 500 mg, from about 75 mg to about 450 mg, from about 75 mg to about 400 mg, from about 75 mg to about 350 mg, from about 75 mg to about 300 mg, from about 75 mg to about 250 mg, from about 75 mg to about 200 mg, from about 75 mg to about 150 mg, from about 75 mg to about 100 mg, from about 300 mg to about 5,000 mg, from about 300 mg to about 4,500 mg, from about 300 mg to about 4,000 mg, from about 300 mg to about 3,500 mg, from about 300 mg to about 3,000 mg, from about 300 mg to about 2,500 mg, from about 300 mg to about 2,000 mg, from about 300 mg to about 1,500 mg, from about 300 mg to about 1,400 mg, from about 300 mg to about 1,300 mg, from about 300 mg to about 1,200 mg, from about 300 mg to about 1,100 mg, from about 300 mg to about 1,000 mg, from about 300 mg to about 900 mg, from about 300 mg to about 800 mg, from about 300 mg to about 700 mg, from about 300 mg to about 600 mg, from about 300 mg to about 550 mg, from about 300 mg to about 500 mg, from about 300 mg to about 450 mg, from about 300 mg to about 400 mg, or from about 300 mg to about 350 mg of Compound 1a or a tautomer thereof. In some embodiments, the therapeutically effective amount is a total daily dosage of from about 40 mg to about 3000 mg, from about 40 mg to about 2000 mg, from about 40 mg to about 1000 mg, from about 75 mg to about 1000 mg, from about 75 mg to about 800 mg, or from about 75 mg to about 600 mg of Compound 1a, when the subject is an adult of at least 18 years old or an adolescent of at least 12 years old with a body weight of at least 50 kg.

In some embodiments, the therapeutically effective amount is a total daily dosage of about 75 mg, about 300 mg, or about 600 mg of Compound 1a, when the subject is an adult of at least 18 years old or an adolescent of at least 12 years old with a body weight of at least 50 kg. In some embodiments, the therapeutically effective amount is a total daily dosage of about 75 mg of Compound 1a, when the subject is an adult of at least 18 years old or an adolescent of at least 12 years old with a body weight of at least 50 kg. In some embodiments, the therapeutically effective amount is a total daily dosage of about 300 mg of Compound 1a, when the subject is an adult of at least 18 years old or an adolescent of at least 12 years old with a body weight of at least 50 kg. In some embodiments, the therapeutically effective amount is a total daily dosage of about 600 mg of Compound 1a, when the subject is an adult of at least 18 years old or an adolescent of at least 12 years old with a body weight of at least 50 kg.

A person of skill in the art would recognize that an adjustment of a therapeutically effective amount of Compound 1 or 1a is necessary, when the subject is a child from 6 to 12 years old or having a body weight of less than 50 kg.

When the subject is a child from 6 to 12 years old or having a body weight of less than 50 kg, in some embodiments, the therapeutically effective amount of Compound 1 or 1a is adjusted to about ½, about ⅓, about ¼, or about ⅕ of a total daily dosage as described herein for the subject being an adult of at least 18 years old or an adolescent of at least 12 years old with a body weight of at least 50 kg. In some embodiments, the therapeutically effective amount of Compound 1 or 1a is adjusted to about ½ of a total daily dosage as described herein for the subject being an adult of at least 18 years old or an adolescent of at least 12 years old with a body weight of at least 50 kg. In some embodiments, the therapeutically effective amount of Compound 1 or 1a is adjusted to about ⅓ of a total daily dosage as described herein for the subject being an adult of at least 18 years old or an adolescent of at least 12 years old with a body weight of at least 50 kg. In some embodiments, the therapeutically effective amount of Compound 1 or 1a is adjusted to about ¼ of a total daily dosage as described herein for the subject being an adult of at least 18 years old or an adolescent of at least 12 years old with a body weight of at least 50 kg. In some embodiments, the therapeutically effective amount of Compound 1 or 1a is adjusted to about ⅕ of a total daily dosage as described herein for the subject being an adult of at least 18 years old or an adolescent of at least 12 years old with a body weight of at least 50 kg. Table 1 shows an example of body weight-based dose adjustment scenarios based on a PopPK model.

TABLE 1
Body weight-based dose adjustment
scenarios based on PopPK model

| | | | |
|---|---|---|---|
| | 75 mg adult | 300 mg adult | 600 mg adult |
| | dose | dose | dose |
| | equivalent | equivalent | equivalent |
| Body weight | (mg) | (mg) | (mg) |

| | | | | |
|---|---|---|---|---|
| <30 | kg | 25 | 100 | 200 |
| 30-50 | kg | 50 | 200 | 400 |
| >50 | kg | 75 | 300 | 600 |

In general, Compound 1 or 1a can be administered orally. In some embodiments, Compound 1, a tautomer, a pharmaceutically acceptable salt, a hydrate, a solvate, or a combination thereof is administered orally. In some embodiments, Compound 1, a tautomer, a pharmaceutically acceptable salt, a hydrate, a solvate, or a combination thereof in a tablet formulation is administered orally. In some embodiments, Compound 1a or a tautomer thereof is administered orally. In some embodiments, Compound 1a or a tautomer thereof in a tablet formulation is administered orally.

The compound (e.g., Compound 1, such as Compound 1a), a tautomer, a pharmaceutically acceptable salt, a hydrate, a solvate, or a combination thereof can be administered once (e.g., q.d.), twice (e.g., b.i.d. or q12 h), three (e.g., t.i.d.), or four times (e.g., q.i.d.) a day. In some embodiments, Compound 1, a tautomer, a pharmaceutically acceptable salt, a hydrate, a solvate, or a combination thereof is administered once daily (i.e., QD). In some embodiments, Compound 1, a tautomer, a pharmaceutically acceptable salt, a hydrate, a solvate, or a combination thereof is administered twice daily (i.e., BID), such as in two doses separated by approximately 12 hours (i.e., Q12H). In some embodiments, Compound 1 or a pharmaceutically acceptable salt, hydrate, solvate, or combination thereof is administered three times daily. In some embodiments, Compound 1, a tautomer, a pharmaceutically acceptable salt, a hydrate, a solvate, or a combination thereof is administered four times daily. In some embodiments, Compound 1, a tautomer, a pharmaceutically acceptable salt, a hydrate, a solvate, or a combination thereof is administered once every other day (i.e., QOD).

In some embodiments, Compound 1a or a tautomer thereof is administered once daily (i.e., QD). In some embodiments, Compound 1a or a tautomer thereof is administered twice daily. In some embodiments, Compound 1a or a tautomer thereof is administered three times daily. In some embodiments, Compound 1a or a tautomer thereof is administered four times daily. In some embodiments, Compound 1a or a tautomer thereof is administered once every other day (i.e., QOD).

In some embodiments, about 40 mg, about 50 mg, about 60 mg, about 70 mg, about 75 mg, about 80 mg, about 90 mg, about 100 mg, about 120 mg, about 150 mg, about 180 mg, about 200 mg, about 250 mg, about 300 mg, about 330 mg, about 350 mg, about 360 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2500 mg, or about 3000 mg of Compound 1, a tautomer, a pharmaceutically acceptable salt, a hydrate, a solvate, or a combination thereof is administered once daily. In some embodiments, about 40 mg of Compound 1, a tautomer, a pharmaceutically acceptable salt, a hydrate, a solvate, or a combination thereof is administered once daily. In some embodiments, about 75 mg of Compound 1, a tautomer, a pharmaceutically acceptable salt, a hydrate, a solvate, or a combination thereof is administered once daily. In some embodiments, about 120 mg of Compound 1, a tautomer, a pharmaceutically acceptable salt, a hydrate, a solvate, or a combination thereof is administered once daily. In some embodiments, about 300 mg of Compound 1, a tautomer, a pharmaceutically acceptable salt, a hydrate, a solvate, or a combination thereof is administered once daily. In some embodiments, about 360 mg of Compound 1, a tautomer, a pharmaceutically acceptable salt, a hydrate, a solvate, or a combination thereof is administered once daily. In some embodiments, about 500 mg of Compound 1, a tautomer, a pharmaceutically acceptable salt, a hydrate, a solvate, or a combination thereof is administered once daily. In some embodiments, about 600 mg of Compound 1, a tautomer, a pharmaceutically acceptable salt, a hydrate, a solvate, or a combination thereof is administered once daily. In some embodiments, about 750 mg of Compound 1, a tautomer, a pharmaceutically acceptable salt, a hydrate, a solvate, or a combination thereof is administered once daily. In some embodiments, about 1000 mg of Compound 1, a tautomer, a pharmaceutically acceptable salt, a hydrate, a solvate, or a combination thereof is administered once daily. In some embodiments, about 2000 mg of Compound 1, a tautomer, a pharmaceutically acceptable salt, a hydrate, a solvate, or a combination thereof is administered once daily.

In some embodiments, about 75 mg of Compound 1a or a tautomer thereof is administered once daily. In some embodiments, about 300 mg of Compound 1a or a tautomer thereof is administered once daily. In some embodiments, about 600 mg of Compound 1a or a tautomer thereof is administered once daily.

In some embodiments, about 35 mg of Compound 1, a tautomer, a pharmaceutically acceptable salt, a hydrate, a solvate, or a combination thereof is administered twice daily. In some embodiments, about 125 mg of Compound 1, a tautomer, a pharmaceutically acceptable salt, a hydrate, a solvate, or a combination thereof is administered twice daily. In some embodiments, about 150 mg of Compound 1, a tautomer, a pharmaceutically acceptable salt, a hydrate, a solvate, or a combination thereof is administered twice daily. In some embodiments, about 300 mg of Compound 1, a tautomer, a pharmaceutically acceptable salt, a hydrate, a solvate, or a combination thereof is administered twice daily. In some embodiments, about 500 mg of Compound 1, a tautomer, a pharmaceutically acceptable salt, a hydrate, a solvate, or a combination thereof is administered twice daily. In some embodiments the twice daily dosages are administered about 12 hours apart.

In some embodiments, Compound 1, a tautomer, a pharmaceutically acceptable salt, a hydrate, a solvate, or a combination thereof is administered for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83 84, 85, 86, 87, 88, 89, 90, 91, 92, 93, 94, 95, 96, 97, 98, 99, 100 days, or longer. In some embodiments, Compound 1, a tautomer, a pharmaceutically acceptable salt, a hydrate, a solvate, or a combination thereof is administered for at least 7, 14, 21, 28, 35, 42, 49, or 56 days. In some embodiments, Compound 1, a tautomer, a pharmaceutically acceptable salt, a hydrate, a solvate, or a combination thereof is administered for at least 28 days. In some embodiments, Compound 1, a tautomer, a pharmaceutically acceptable salt, a hydrate, a solvate, or a combination thereof is administered for at least 56 days. In some embodiments, Compound 1, a tautomer, a pharmaceutically acceptable salt, a hydrate, a solvate, or a combination thereof for at least 84 days.

In some embodiments, Compound 1, a tautomer, a pharmaceutically acceptable salt, a hydrate, a solvate, or a combination thereof is administered for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more months. In some embodiments, Compound 1, a tautomer, a pharmaceutically acceptable salt, a hydrate, a solvate, or a combination thereof is administered for at least 10, 15, 20, 25, 30, 35, 40, 45, or 50 months. In some embodiments, Compound 1, a tautomer, a pharmaceutically acceptable salt, a hydrate, a solvate, or a combination thereof is administered for at least 6 months. In some embodiments, Compound 1, a tautomer, a pharmaceutically acceptable salt, a hydrate, a solvate, or a combination thereof is administered for at least 12 months. In some embodiments, Compound 1, a tautomer, a pharmaceutically acceptable salt, a hydrate, a solvate, or a combination thereof is administered for at least 18 months. In some embodiments, Compound 1, a tautomer, a pharmaceutically acceptable salt, a hydrate, a solvate, or a combination thereof is administered for at least 24 months. In some embodiments, Compound 1, a tautomer, a pharmaceutically acceptable salt, a hydrate, a solvate, or a combination thereof is administered for at least 30 months. In some embodiments, Compound 1, a tautomer, a pharmaceutically acceptable salt, a hydrate, a solvate, or a combination thereof is administered for at least 36 months. In some embodiments, Compound 1, a tautomer, a pharmaceutically acceptable salt, a hydrate, a solvate, or a combination thereof is administered for at least 42 months. In some embodiments, Compound 1, a tautomer, a pharmaceutically acceptable salt, a hydrate, a solvate, or a combination thereof is administered for the lifetime of the subject.

In some embodiments, Compound 1a or a tautomer thereof is administered for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83 84, 85, 86, 87, 88, 89, 90, 91, 92, 93, 94, 95, 96, 97, 98, 99, 100 days or longer. In some embodiments, Compound 1a or a tautomer thereof is administered for at least 7, 14, 21, 28, 35, 42, 49, or 56 days. In some embodiments, Compound 1a or a tautomer thereof is administered for at least 28 days.

In some embodiments, Compound 1a or a tautomer thereof is administered for 28 days during each of the single treatment period of Part A, the single treatment period of Part B, and the extension period of Part B, as described in Section III-3. Treatment Period.

In some embodiments, Compound 1a or a tautomer thereof is administered for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more months. In some embodiments, Compound 1a or a tautomer thereof is administered for at least 10, 15, 20, 25, 30, 35, 40, 45, or 50 months. In some embodiments, Compound 1a or a tautomer thereof is administered for at least 6 months. In some embodiments, Compound 1a or a tautomer thereof is administered for at least 12 months. In some embodiments, Compound 1a or a tautomer thereof is administered for at least 18 months. In some embodiments, Compound 1a or a tautomer thereof is administered for at least 24 months. In some embodiments, Compound 1a or a tautomer thereof is administered for at least 30 months. In some embodiments, Compound 1a or a tautomer thereof is administered for at least 36 months. In some embodiments, Compound 1a or a tautomer thereof is administered for at least 42 months. In some embodiments, Compound 1a or a tautomer thereof is administered for the lifetime of the subject.

In some embodiments, Compound 1a or a tautomer thereof is administered for about 12 months during the extension period of Part A treatment as described herein. In some embodiments, Compound 1a or a tautomer thereof is administered for about 36 months during the long-term extension (LTE) period of the treatment as described in Section III-3. Treatment Period.

### III-5: Efficacy

A Two-Part Multinational, Randomized, Placebo-controlled Trial can evaluate the safety and efficacy of Compound 1a for the treatment of children (≥6 years of age) and adults with primary hyperoxaluria type 1 (PH1), as described in Example 2.

Administration of a therapeutically effective amount of Compound 1 or 1a may reduce or substantially eliminate one or more symptoms associated with primary hyperoxaluria type 1 (PH1) in subjects.

In some embodiments, the therapeutically effective amount of Compound 1, a tautomer, a pharmaceutically acceptable salt, a hydrate, a solvate, or a combination thereof reduces the uOxalate excretion to less than about 0.46 mmol/1.73 m² per 24 hours. In some embodiments, the therapeutically effective amount of Compound 1a or a tautomer thereof reduces the uOxalate excretion to less than about 0.46 mmol/1.73 m² per 24 hours.

In some embodiments, the therapeutically effective amount of Compound 1, a tautomer, a pharmaceutically acceptable salt, a hydrate, a solvate, or a combination thereof reduces the uOxalate excretion by at least about 50% relative to a uOxalate excretion prior to the treating. In some embodiments, the therapeutically effective amount of Compound 1a or a tautomer thereof reduces the uOxalate excretion by from about 50% relative to a uOxalate excretion prior to the treating.

In some embodiments, the therapeutically effective amount of Compound 1, a tautomer, a pharmaceutically acceptable salt, a hydrate, a solvate, or a combination thereof brings a ratio of uOxalate to urine creatinine (uCreatinine) to a normal range of less than about 63.7 mg/mg, which is 80 mmol/mmol. In some embodiments, the therapeutically effective amount of Compound 1a or a tautomer thereof brings a ratio of uOxalate to urine creatinine (uCreatinine) to a normal range of less than about 63.7 mg/mg, which is 80 mmol/mmol.

In some embodiments, the therapeutically effective amount of Compound 1, a tautomer, a pharmaceutically acceptable salt, a hydrate, a solvate, or a combination thereof reduces a plasma glycolate (pGlycolate) concentration in an absolute amount and/or in a relative amount compared to a pGlycolate concentration prior to the treating. In some embodiments, the therapeutically effective amount of Compound 1a or a tautomer thereof reduces a plasma glycolate (pGlycolate) concentration in an absolute amount and/or in a relative amount compared to a pGlycolate concentration prior to the treating.

In some embodiments, the therapeutically effective amount of Compound 1, a tautomer, a pharmaceutically acceptable salt, a hydrate, a solvate, or a combination thereof increases an estimated glomerular filtration rate (eGFR) in an absolute amount and/or in a relative amount compared to an eGFR prior to the treating. In some embodiments, the therapeutically effective amount of Compound 1a or a tautomer thereof increases an estimated glomerular filtration rate (eGFR) in an absolute amount and/or in a relative amount compared to an eGFR prior to the treating.

In some embodiments, the therapeutically effective amount of Compound 1, a tautomer, a pharmaceutically acceptable salt, a hydrate, a solvate, or a combination thereof reduces a size of the kidney stones. In some embodiments, the therapeutically effective amount of Compound 1a or a tautomer thereof reduces a size of the kidney stones.

In some embodiments, the therapeutically effective amount of Compound 1, a tautomer, a pharmaceutically acceptable salt, a hydrate, a solvate, or a combination thereof prevents the development, worsening, or recurrence of kidney stones. In some embodiments, the therapeutically effective amount of Compound 1a or a tautomer thereof prevents the development, worsening, or recurrence of kidney stones.

In some embodiments, the subject is further evaluated to by one or more tests (e.g., tests according to Table 4, Table 5, and Table 6 of Example 2) to provide overall assessments including plasma pharmacokinetic and/or pharmacodynamic profiles. Examples of such tests are described in, e.g., Table 4, Table 5, and Table 6 of Example 2.

### IV. Compositions

Compound 1, a tautomer, a pharmaceutically acceptable salt, a hydrate, a solvate, or a combination thereof (such as Compound 1a) can be prepared in various compositions suitable for delivery to a subject. A composition suitable for administration to a subject typically comprises Compound 1, a tautomer, a pharmaceutically acceptable salt, a hydrate, a solvate, or a combination thereof and a pharmaceutically acceptable excipient.

The pharmaceutical compositions can be in the form of a sterile injectable aqueous or oleaginous solutions and suspensions. Sterile injectable preparations can be formulated using non-toxic parenterally-acceptable vehicles including water, Ringer's solution, and isotonic sodium chloride solution, and acceptable solvents such as 1,3-butane diol. In addition, sterile, fixed oils are conventionally employed as a solvent or suspending medium. For this purpose, a bland fixed oil such as a synthetic mono- or diglyceride may be used. In addition, fatty acids such as oleic acid may be useful in the preparation of injectables.

Aqueous suspensions contain the active ingredient in admixture with excipients suitable for the manufacture of aqueous suspensions. Such excipients include, but are not limited to, suspending agents such as sodium carboxymethylcellulose, methylcellulose, oleagino-propylmethylcellulose, sodium alginate, polyvinyl-pyrrolidone, gum tragacanth and gum acacia; dispersing or wetting agents such as lecithin, polyoxyethylene stearate, and polyethylene sorbitan monooleate; and preservatives such as ethyl, n-propyl, and p-hydroxybenzoate.

Oily suspensions can be formulated by suspending the active ingredient in a vegetable oil such as, for example, arachis oil, olive oil, sesame oil or coconut oil, or in a mineral oil such as liquid paraffin. The oily suspensions can contain a thickening agent, such as beeswax, hard paraffin, or cetyl alcohol. These compositions can be preserved by the addition of an antioxidant such as ascorbic acid.

Dispersible powders and granules (suitable for preparation of an aqueous suspension by the addition of water) can contain the active ingredient in admixture with a dispersing agent, wetting agent, suspending agent, or combinations thereof. Additional excipients can also be present.

The pharmaceutical compositions including Compound 1, a tautomer, a pharmaceutically acceptable salt, a hydrate, a solvate, or a combination thereof (e.g., Compound 1a) can also be in the form of oil-in-water emulsions. The oily phase can be a vegetable oil such as, for example olive oil or arachis oil, or a mineral oil such as, for example, liquid paraffin, or mixtures of these. Suitable emulsifying agents include, for example, naturally-occurring gums, such as gum acacia and gum tragacanth; naturally-occurring phospholipids such as soy lecithin; esters or partial esters derived from fatty acids and hexitol anhydrides, such as sorbitan monooleate; and condensation products of said partial esters with ethylene oxide, such as polyoxyethylene sorbitan monooleate.

Pharmaceutical compositions containing including Compound 1, a tautomer, a pharmaceutically acceptable salt, a hydrate, a solvate, or a combination thereof (e.g., Compound 1a) can also be in a form suitable for oral use. Suitable compositions for oral administration include, but are not limited to, tablets, troches, lozenges, aqueous or oily suspensions, dispersible powders or granules, emulsions, hard or soft capsules, syrups, elixirs, solutions, buccal patches, oral gels, chewing gums, chewable tablets, effervescent powders, and effervescent tablets. Compositions for oral administration can be formulated according to any method known to those of skill in the art. Such compositions can contain one or more agents selected from sweetening agents, flavoring agents, coloring agents, antioxidants, and preserving agents in order to provide pharmaceutically elegant and palatable preparations.

Tablets generally contain the active ingredient in admixture with non-toxic pharmaceutically acceptable excipients, including, for example, inert diluents, such as cellulose, silicon dioxide, aluminum oxide, calcium carbonate, sodium carbonate, glucose, mannitol, sorbitol, lactose, calcium phosphate, and sodium phosphate; granulating and disintegrating agents, such as corn starch and alginic acid; binding agents, such as polyvinylpyrrolidone (PVP), cellulose, polyethylene glycol (PEG), starch, gelatin, and acacia; and lubricating agents such as magnesium stearate, stearic acid, and talc. The tablets can be uncoated or coated, enterically or otherwise, by known techniques to delay disintegration and absorption in the gastrointestinal tract and thereby provide a sustained action over a longer period. For example, a time delay material such as glyceryl monostearate or glyceryl distearate can be employed. Tablets can also be coated with a semi-permeable membrane and optional polymeric osmogents according to known techniques to form osmotic pump compositions for controlled release.

Compositions for oral administration can be formulated as hard gelatin capsules wherein the active ingredient is mixed with an inert solid diluent (such as calcium carbonate, calcium phosphate, or kaolin), or as soft gelatin capsules wherein the active ingredient is mixed with water or an oil medium (such as peanut oil, liquid paraffin, or olive oil).

Transdermal delivery of Compound 1, a tautomer, a pharmaceutically acceptable salt, a hydrate, a solvate, or a combination thereof (e.g., Compound 1a) can be accomplished by means of iontophoretic patches and the like. The compound can also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols.

In some embodiments, the pharmaceutical composition includes Compound 1, a tautomer, a pharmaceutically acceptable salt, a hydrate, a solvate, or a combination thereof (e.g., Compound 1a) as described herein and one or more additional active agents for treating kidney stones. Examples of such active agents include, but are not limited to, thiazides (e.g., bendroflumethiazide, chlorothiazide, chlorthalidone, hydrochlorothiazide, indapamide, methyclothiazide, metolazone, polythiazide, and the like); citrate salts (e.g., sodium citrate, potassium citrate, and the like); phosphate salts (e.g., monopotassium phosphate, dipotassium phosphate, and the like); vitamin B₆ compounds (e.g., pyridoxine, pyridoxal, pyridoxamine, and the like); cystine-binding thiol compounds (e.g., α-mercaptopropionylglycine, D-penicillamine, captopril, and the like); purine analog xanthine oxidase inhibitors (e.g., allopurinol, oxypurinol, and the like); and other xanthine oxidase inhibitors (e.g., febuxostate, topiroxostat, and the like).

### V. Kits

The disclosure also encompasses kits comprising pharmaceutical compositions and dosage forms including Compound 1, a tautomer, a pharmaceutically acceptable salt, a hydrate, a solvate, or a combination thereof (e.g., Compound 1a).

In some aspects, the present disclosure provides a kit that includes, a tautomer, a pharmaceutically acceptable salt, a hydrate, a solvate, or a combination thereof (e.g., Compound 1a). Some of the kits described herein include a label describing a method of administering Compound 1, a tautomer, a pharmaceutically acceptable salt, a hydrate, a solvate, or a combination thereof (e.g., Compound 1a). Some of the kits described herein include a label describing a method of treating hyperoxaluria. In some embodiments, the kits described herein include a label describing a method of treating kidney stones. In some embodiments, the kit includes Compound 1a or a tautomer thereof.

The compositions of the present disclosure, including but not limited to, compositions including Compound 1, a tautomer, a pharmaceutically acceptable salt, a hydrate, a solvate, or a combination thereof (e.g., Compound 1a) in a bottle, jar, vial, ampoule, tube, blister pack, or other container-closure system approved by the Food and Drug Administration (FDA) or other regulatory body, which may provide one or more dosages containing the compounds. The package or dispenser may also be accompanied by a notice associated with the container in a form prescribed by a governmental agency regulating the manufacture, use, or sale of pharmaceuticals, the notice indicating approval by the agency. In certain aspects, the kit may include a formulation or composition as described herein, a container closure system including the formulation or a dosage unit form including the formulation, and a notice or instructions describing a method of use as described herein.

### VI. List of Abbreviations

| Abbreviation | |
|---|---|
| or Term | Definition/Explanation |
| ADL | Activities of daily living |
| AE | Adverse event |
| AGT | Alanine glyoxylate aminotransferase (enzyme name) |
| AGXT | Alanine-glyoxylate aminotransferase (gene name) |
| ALP | Alkaline phosphatase |
| ALT | Alanine aminotransferase |
| ANCOVA | Analysis of covariance |
| ANOVA | Analysis of variance |
| AST | Aspartate aminotransferase |
| AUC | Area under the concentration time curve |
| AxMP | Auxiliary medicinal product |
| β-hCG | Beta human chorionic gonadotropin |
| BUN | Blood urea nitrogen |
| CaOx | Calcium oxalate |
| CF | Correction factor |
| CFR | Code of Federal Regulations |
| CIOMS | Council for International Organizations of |
| | Medical Sciences |
| CKD-EPIcr_R | Chronic Kidney Disease Epidemiology Collaboration |
| | creatinine equation refit without the race variable |
| Cₘₐₓ | Maximum concentration |
| CONSORT | Consolidated Standards of Reporting Trials |
| CRF | Case Report Form |
| CSR | Clinical Study Report |
| CTFG | Clinical Trials Facilitation Group |
| CV | Clinic visit |
| DMC | Data Monitoring Committee |
| DS | Drug substance |
| ECG | Electrocardiogram |
| eCRF | Electronic Case Report Form |
| EDC | Electronic data capture |
| eGFR | Estimated glomerular filtration rate |
| EoS | End of study |
| ESRD | End-stage renal disease |
| ET | Early termination |
| FAS | Full analysis set |
| FIH | First in human |
| FSH | Follicle stimulating hormone |
| F-U | Follow-up |
| GCP | Good Clinical Practice |
| GO | Glycolate oxidase |
| HbsAg | Hepatitis B surface antigen |
| Hct | Hematocrit |
| HCV | Hepatitis C virus |
| Hgb | Hemoglobin |
| HH | Home health visit |
| HIV | Human immunodeficiency virus |
| IB | Investigator's Brochure |
| IC₅₀ | Half-maximal Inhibitory concentration |
| IC₉₀ | 90% Inhibitory concentration |
| IC₉₅ | 95% Inhibitory concentration |
| ICF | Informed Consent Form |
| ICH | International Council for Harmonisation |
| IEC | Independent Ethics Committee |
| IMP | Investigational medicinal product |
| INR | International normalized ratio |
| IRB | Institutional Review Board |
| IRT | Interactive response technology |
| IUD | Intrauterine device |
| KDQOL | Kidney disease quality of life questionnaire |
| LTE | Long-term extension |
| NF | National Formulary |
| NIMP | Non-investigational medicinal product |
| OHF | Oxalosis and Hyperoxaluria Foundation |
| PD | Pharmacodynamic(s) |
| PedsQL | Pediatric Quality of Life Inventory |
| pGlycolate | Plasma glycolate |
| PH | Primary hyperoxaluria |
| PH1 | Primary hyperoxaluria type 1 |
| Ph Eur | European Pharmacopoeia |
| PK | Pharmacokinetic(s) |
| PK/PD | Pharmacokinetic/Pharmacodynamic |
| pOxalate | Plasma oxalate |
| PR | Time from atrial depolarization to ventricular |
| | depolarization |
| PT | Thromboplastin time |
| PTT | Partial thromboplastin time |
| QD | Once a day |
| QOL | Quality of life |
| QRS | a combination of the Q wave, R wave and S wave |
| QT interval | The portion of an electrocardiogram between the onset |
| | of the Q wave and the end of the T wave. |
| QTc | Corrected QT interval |
| QTcB | Corrected QT interval using Bazett's formula |
| QTcF | Corrected QT interval using Fridericia's formula |
| QTL | Quality tolerance limit |
| RBC | Red blood cells |
| REB | Research Ethics Board |
| RNA | Ribonucleic acid |
| RNAi | Ribonucleic acid interference |
| RWP | Randomized withdrawal period |
| SAE | Serious adverse event |
| SAP | Statistical analysis plan |
| SARS-CoV-2 | Severe acute respiratory syndrome coronavirus 2 |
| SCr | Standardized serum creatinine |
| SGOT | Serum glutamic-oxaloacetic transaminase |
| SGPT | Serum glutamic pyruvic transaminase |
| SoA | Schedule of assessments |
| TBL | Total bilirubin |
| TC | Telephone call |
| Tₘₐₓ | Time to reach maximum concentration |
| TMF | Trial Master File |
| uCreatinine | Urinary creatinine |
| uGlycolate | Urinary glycolate |
| ULN | Upper limit of normal |
| uOxalate | Urinary oxalate |
| US | United States |
| USP | United States Pharmacopeia |
| WBC | White blood cell |
| WOCBP | Women of childbearing potential |

### VII. Examples

The following examples are offered to illustrate, but not to limit, the claimed invention(s).

### Example 1: Phase 1 Clinical Study

A Phase 1 clinical study is performed to assess the safety and dosing of Compound 1a. This Phase 1 study consists of two parts. In Part A, healthy adults randomized 3:1 (active:placebo) receive a single dose of orally administered Compound 1a or matching placebo in six (6) ascending dose groups. In Part B, healthy adults randomized 3:1 (active:placebo) received multiple doses of orally administered Compound 1a or matching placebo in six (6) ascending dose groups. Safety and tolerability were assessed by the incidence of adverse events, vital signs, electrocardiograms, and clinical laboratory tests. Pharmacokinetics and pharmacodynamics (changes in plasma and urine glycolate) were measured using validated bioanalytical assays.

Study Design: The study was approved by the Advarra Institutional Review Board (Columbia, MD). All participants provided written informed consent. The trial was conducted at a single center (Celerion, Tempe, Arizona) according to the Good Clinical Practice guidelines of the International Conference on Harmonization and the World Health Organization Declaration of Helsinki. The study was a randomized, double-blind, placebo-controlled, single and multiple ascending dose study. The primary objective of the study was to evaluate the safety and tolerability of single and multiple doses of Compound 1a administered to healthy adult volunteers. The secondary objectives were to characterize the pharmacokinetics (PK) of Compound 1a and to describe the pharmacodynamic (PD) properties in healthy adult volunteers. The study also evaluated the effect of food on the PK of Compound 1a.

Participants: Healthy adult volunteers ages 18 to 55 were eligible for the study.

Intervention: Compound 1a was provided as a compounded oral suspension of 10 milligrams per milliliter (mg/mL) and 40 mg/mL strengths. The compounded oral drug product was administered to subjects using polypropylene oral dosing syringes. Both oral suspensions are prepared by dispersing Compound 1a in Purified Water (USP) in a mortar and pestle followed by successive rinses of an oral suspending vehicle sold under the name ORA-Plus® and a sugar-free oral syrup vehicle sold under the name ORA-Sweet® SF to volume to generate the required concentration of Compound 1a. The Compound 1a oral suspensions, 10 mg/mL and 40 mg/mL were prepared at the compounding pharmacy. The vehicles sold under the names ORA-Plus® and ORA-Sweet® SF are mixtures of compendial excipients formulated to assist the compounding pharmacist in the preparation of extemporaneous delivery systems.

Pharmacokinetics: Human K2-ethylenediaminetetraacetic acid (K2-EDTA) plasma samples were analyzed for Compound 1a by a validated method over a range of 1.0 to 1000 ng/mL based on the analysis of 100 μL of sample. Human plasma containing Compound 1a and the internal standard, Compound 1a-D₄, was extracted using liquid-liquid extraction and analyzed by a Sciex API 4000 LC-MS-MS equipped with an HPLC column. The peak area of the m/z 370.2→298.0 Compound 1a product ion was measured against the peak area of the m/z 374.3→302.4 Compound 1a-D₄ internal standard product ion. Quantitation was performed using a weighted 1/x² linear least squares regression analysis generated from calibration standards prepared on the day of extraction.

Acetonitrile fortified human urine samples were analyzed for Compound 1a by a validated method over a range of 0.500 to 500 ng/mL based on the analysis of 100 μL of sample. Human urine containing Compound 1a and the internal standard, Compound 1a-D₄, was extracted using liquid-liquid extraction and analyzed by a Sciex API 4000 LC-MS-MS equipped with an HPLC column. The peak area of the m/z 370.2-298.0 Compound 1a product ion was measured against the peak area of the m/z 374.3-302.4 Compound 1a-D₄ internal standard product ion. Quantitation was performed using a weighted 1/x² linear least squares regression analysis generated from calibration standards prepared on the day of extraction.

Pharmacodynamics: Acidified human K2-EDTA plasma samples were analyzed for oxalic acid and glycolic acid by a validated method over a range of 8.00 to 120 μM for oxalic acid and 5.00 to 625 μM for glycolic acid based on the analysis of 50 μL of sample. Human plasma containing oxalic acid, glycolic acid and internal standards, ¹³C2-oxalic acid and D2-glycolic acid, was derivatized after protein precipitation then extracted using liquid-liquid extraction and analyzed by a Sciex API 4000 LC-MS-MS equipped with an HPLC column. The peak area of the m/z 224.1→152.1 oxalic acid product ion was measured against the peak area of the m/z 226.1→152.1 13C2-oxalic acid internal standard product ion. The peak area of the m/z 210.1→107.0 glycolic acid product ion was measured against the peak area of the m/z 212.1→107.0 D2-glycolic acid internal standard product ion. Quantitation was performed using a weighted 1/x2 linear least squares regression analysis generated from calibration standards prepared on the day of extraction.

Acidified human urine samples were analyzed for oxalic acid and glycolic acid by a validated method over a range of 50.0 to 10,000 μM for oxalic acid and 25.0 to 5000 μM for glycolic acid based on the analysis of 20 μL of sample. Human urine containing oxalic acid, glycolic acid and internal standards, ¹³C2-oxalic acid and D2-glycolic acid, was extracted by creating hydrazide derivatives of the analyte, evaporating to dryness and reconstituting before being analyzed by a Sciex API 4000 LC-MS-MS equipped with an HPLC column. The peak area of the m/z 224.1→152.1 oxalic acid product ion was measured against the peak area of the m/z 226.1→152.1 13C2-oxalic acid internal standard product ion. The peak area of the m/z 210.1→137.0 glycolic acid product ion was measured against the peak area of the m/z 212.1→137.0 D2-glycolic acid internal standard product ion. Quantitation was performed using a weighted 1/x2 linear least squares regression analysis generated from calibration standards prepared on the day of extraction.

Human urine samples were analyzed for creatinine by a validated method over a range of 10 to 2000 μg/mL based on the analysis of 50 μL of sample. Human urine containing creatinine and the internal standard, creatinine-D3, was extracted using direct dilution and analyzed by a Sciex API 4000 LC-MS-MS equipped with an HPLC column. The peak area of the m/z 114.0-86.0 creatinine product ion was measured against the peak area of the m/z 117.0-89.0 creatinine-D3 internal standard product ion. Quantitation was performed using a weighted 1/x2 linear least squares regression analysis generated from calibration standards prepared on the day of extraction.

Pharmacokinetic analysis: Pharmacokinetic calculations based on Compound 1a plasma concentrations were performed using noncompartmental analysis in Phoenix WinNonlin (Certara, Princeton, New Jersey). Pharmacokinetic parameters calculated from plasma Compound 1a concentrations included, but were not limited to, Cmax, time to maximum concentration (Tmax), area under the plasma concentration-time curve from zero to 12 hr (AUC0-12), area under the plasma concentration-time curve from zero to 24 hr (AUC0-24), and AUC0-inf Calculated PK parameters also included apparent terminal half-life (t½), determined by linear regression of log concentration on the terminal portion of the plasma concentration-time curve and calculated as ln(2)/(−β), where β is the slope of the terminal portion of the log concentration-time curve. Accumulation ratio based on Cmax was calculated as (Cmax,day 7)/(Cmax, day 1).

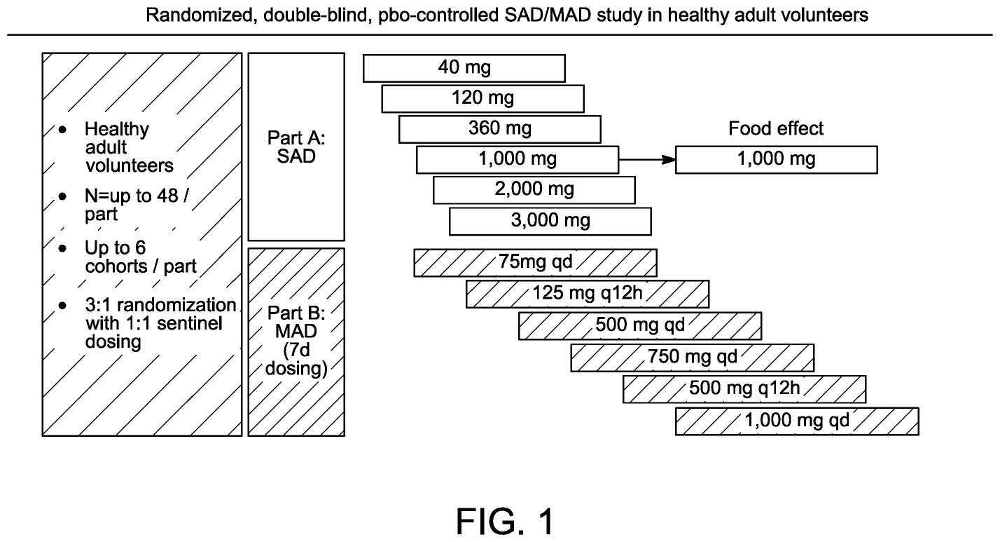

Subject disposition: Six cohorts of 6 to 8 subjects each (N=up to 48) were administered single doses of 40, 120, 360, 1,000, 2,000, and 3,000 mg of Compound 1a in Part A under fasting conditions. Following a 2-week washout, subjects in the 1,000 mg cohort were administered a second 1,000 mg single dose following a high-fat breakfast. Six cohorts of 8 healthy subjects each were administered multiple doses of 75 mg twice daily (qd), 125 mg every 12 hours (q12 h), 500 mg q12 h, 500 mg qd, 750 mg qd, and 1,000 mg qd for 7 days in the MAD Part B (FIG. 1).

Safety: No dose-limiting toxicities were observed in the study. No serious or severe AEs were reported (Table 2). Most AEs were reported by single subjects, and all were mild to moderate in intensity. There were no AEs leading to study discontinuation.

TABLE 2
Adverse Events for Part A and Part B

| | Part A: Single Ascending Dose | | | | |
|---|---|---|---|---|---|
| | 40 mg | 120 mg | 360 mg | 1,000 mg | 2,000 mg |
| Dose | (n = 7) | (n = 8) | (n = 6) | (n = 8) | (n = 8) |
| SAEs | 0 | 0 | 0 | 0 | 0 |
| AEs | 10 | 1 | 0 | 4 | 3 |

| | Part B: Multiple Ascending Dose | | |
|---|---|---|---|
| | 125 mg q12 h | 500 mg q12 h | 500 mg qd |
| Dose | (n = 8) | (n = 8) | (n = 8) |
| SAEs | 0 | 0 | 0 |
| AEs | 6 | 12 | 1 |

Small fluctuations in mean vital sign values were seen in the Compound 1a dose groups and in the pooled placebo group, which were considered expected and clinically insignificant. There were no clinically significant changes or trends observed in safety laboratory tests, nor were there clinically important post dose electrocardiogram findings in the study.

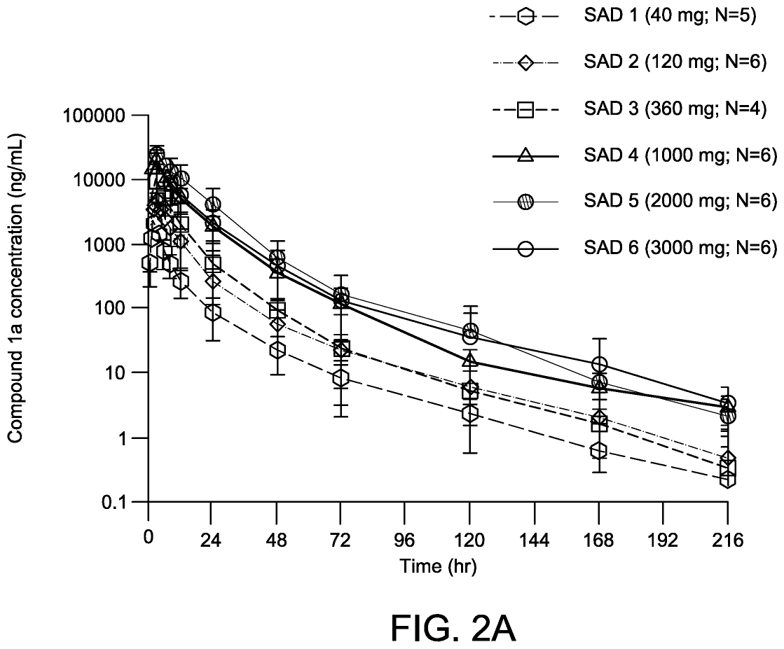

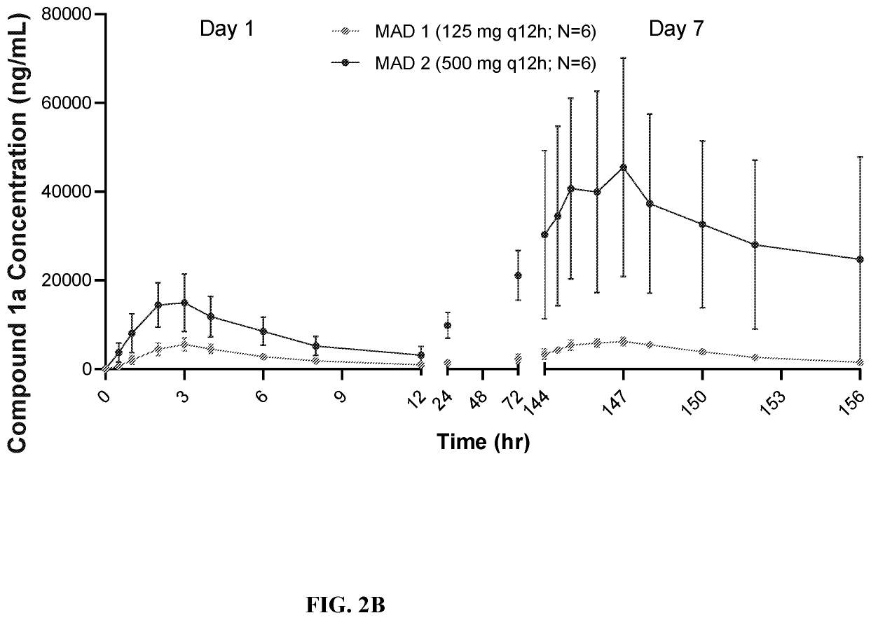

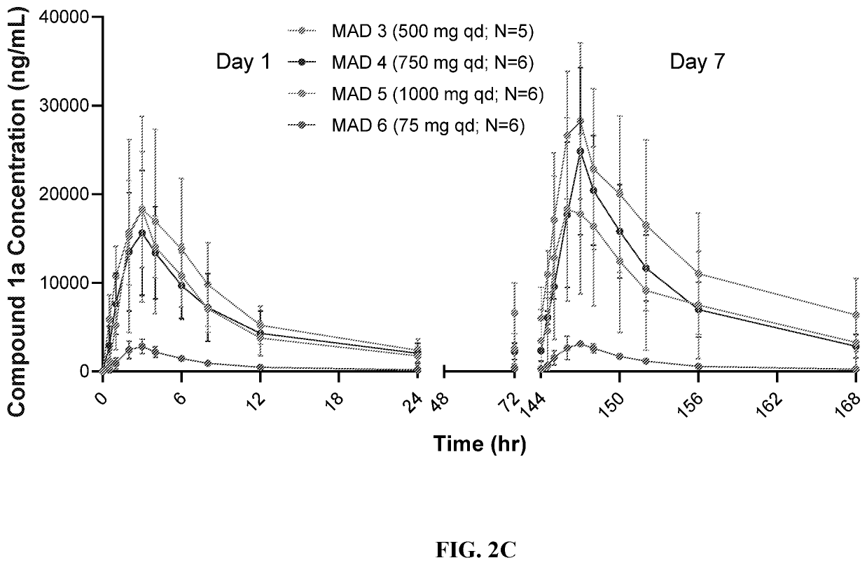

Pharmacokinetics of Compound 1a: Concentration-time profiles for cohorts in the SAD and MAD parts of the study are presented in FIGS. 2A-2C. After single-dose administration of 40 mg to 3,000 mg, average plasma Compound 1a Tₘₐₓ values ranged from 1.9 to 2.9 hours across dose levels. The average t_{1/2} was similar across dose levels, ranging from 21.9 to 33.7 hours.

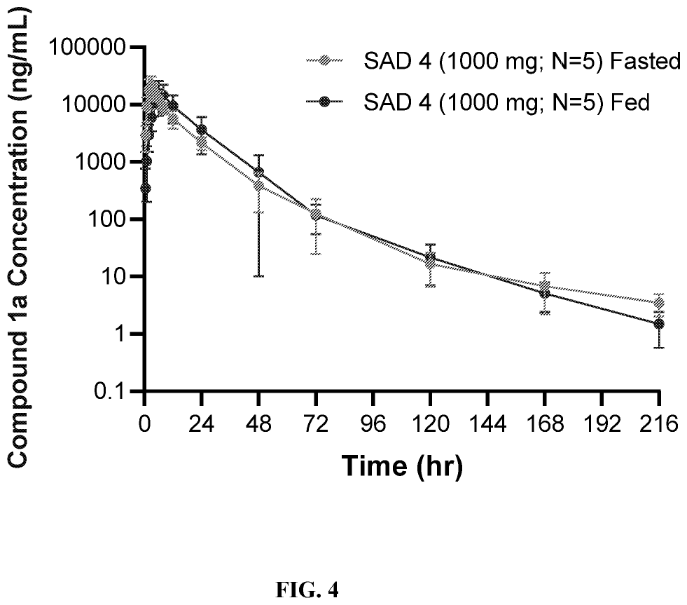

The effect of food intake on the PK of Compound 1a was studied at a single dose of 1,000 mg of Compound a in the same cohort of subjects under fasted and fed conditions sequentially with a 2-week washout between periods. Overall exposure to Compound 1a was slightly lower under fed than under fasted conditions (FIG. 4). There was a delay in Compound 1a Tₘₐₓ under fed in comparison to fasted conditions (average Tₘₐₓ of 5.9 vs 3.0 hours, respectively). The t_{1/2} were approximately similar at 25.2 and 26.8 hours under fed and fasted conditions, respectively. The findings from the fasted vs fed data in the food effect cohort were not considered to be clinically meaningful. Selected PK parameters are summarized in Table 3A and Table 3B.

TABLE 3A
Average Plasma Pharmacokinetic Parameters of Compound 1a (Preliminary Data)

| Parameters | Fasted | Fasted | Fasted | Fasted | Fed | Fasted | Fasted |
|---|---|---|---|---|---|---|---|
| SAD | | | | | | | |
| Dose (mg) | 40 | 120 | 360 | 1,000 | 1,000 | 2,000 | 3,000 |
| N | 5 | 6* | 4 | 6 | 5 | 6 | 6 |
| Cₘₐₓ (ng/mL) | 2040 | 5020 | 10500 | 19500 | 14700 | 25600 | 22500 |
| Tₘₐₓ (hr) | 1.89 | 2.29 | 2.28 | 3.00 | 5.86 | 2.94 | 2.94 |
| t_{1/2} (hr) | 24.6 | 21.9 | 25.9 | 26.8 | 25.2 | 23.4 | 33.7 |
| AUC₀₋₂₄ (ng*hr/mL) | 12100 | 36700 | 74100 | 174000 | 180000 | 253000 | 184000 |
| AUC_{0-inf} (ng*hr/mL) | 14000 | 41400 | 83300 | 214000 | 237000 | 312000 | 226000 |
| MAD | | | | | | | |
| Dose (mg) | 75 | 125 | 500 | 500 | 750 | 1000 | |
| | qd | q12h | q12h | qd | qd | qd | |
| N | 6 | 6 | 6 | 5 | 6 | 6 | |
| Day 7 Cₘₐₓ (ng/mL) | 3420 | 6450 | 43800 | 17000 | 24300 | 29900 | |
| Day 7 Tₘₐₓ (hr) | 2.45 | 2.04 | 1.44 | 2.35 | 2.62 | 2.18 | |
| Day 7 AUC₀₋₁₂ | — | 45600 | 342000 | — | — | — | |
| (ng*hr/mL) | | | | | | | |
| Day 7 AUC₀₋₂₄ | 23900 | — | — | 133000 | 214000 | 303000 | |
| (ng*hr/mL) | | | | | | | |
| AR, Cₘₐₓ | 1.2 | 1.1 | 3.0 | 0.9 | 1.5 | 1.6 | |

*N = 5 for t½

TABLE 3B
Pharmacokinetic Parameters of Compound
1a in Healthy Volunteers

| | | t_{1/2} | Tₘₐₓ | Cₘₐₓ | | AUCₗₐₛₜ | |
|---|---|---|---|---|---|---|---|
| Dose | | (hr) | (hr) | (ng/mL) | | (hr*ng/mL) | |
| 40 | mg | 25 (48%) | 1.9 (41%) | 2,040 | (27%) | 13,900 | (50%) |
| 120 | mg | 22 (72%) | 2.3 (21%) | 5,020 | (38%) | 41,000 | (45%) |
| 360 | mg | 26 (39%) | 2.3 (59%) | 10,500 | (27%) | 83,200 | (41%) |
| 1000 | mg | 28 (38%) | 2.5 (47%) | 17,200 | (62%) | 184,000 | (51%) |
| 2000 | mg | 23 (29%) | 2.9 (42%) | 25,600 | (29%) | 312,000 | (60%) |
| 3000 | mg | 34 (47%) | 2.9 (22%) | 22,500 | (40%) | 225,000 | (39%) |

Geometric mean (geometric CV %)
Accumulation ratios with QD MAD dosing (7 d dosing) were <2

Following both the first dose (day 1) and the final repeat dose (day 7) administration of 125 mg q12 h, 500 mg q12 h, 500 mg qd, mean Compound 1a concentrations peaked at 0.5 to 4 hours after dosing and then declined. Mean accumulation ratios for plasma Compound 1a Cₘₐₓ ranged from 1.1 to 2.9.

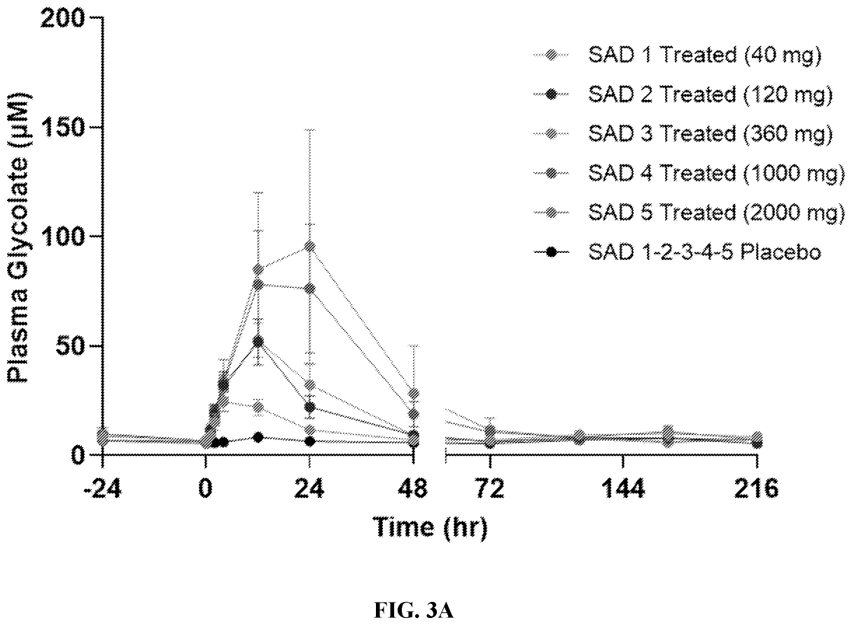

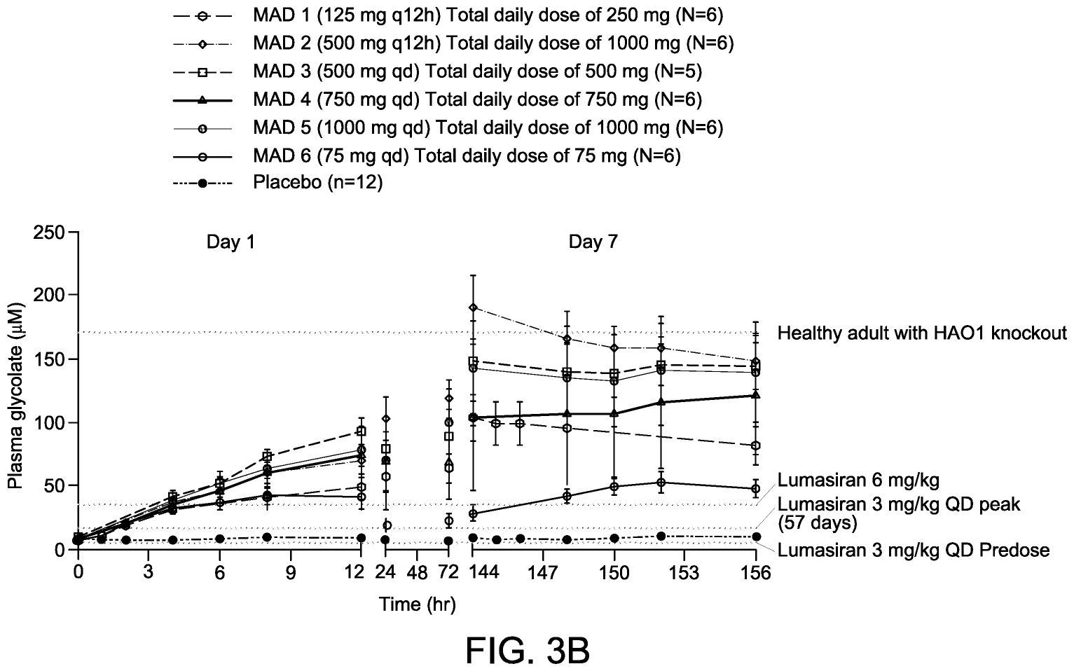

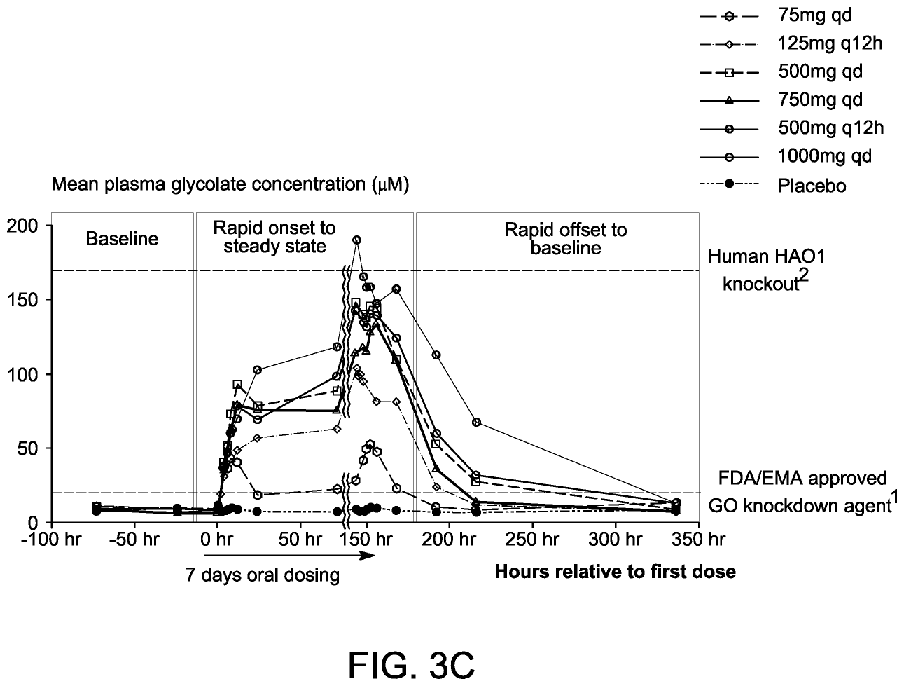

Pharmacodynamics of Compound 1a: As a measure of target engagement, plasma glycolate levels were assessed in Part A and Part B. In Part A, healthy adult volunteers received single doses of Compound 1a at 40, 120, 360, 1,000, 2,000, and 3,000 mg with increases in plasma glycolate observed (FIG. 3A). At all doses, mean maximal plasma glycolate concentrations exceeded the upper limit of normal (ULN) of 14 μM, reaching a mean maximum concentration of >90 μM at doses above 1,000 mg, which was sustained for 24 hours. Median time to recovery (plasma glycolate less than or equal to ULN) after a single dose was less than 72 hours.

In Part B, healthy adult volunteers received multiple doses of Compound 1a at 75 mg qd, 125 mg q12 h, 500 mg q12 h, 500 mg qd, 750 mg qd, and 1000 mg qd. As with Part A, mean maximal plasma glycolate concentrations exceeded the ULN at all doses on day 1 to a range of 50 to 100 μM and further increased on day 7 to a range of 100 to 200 μM. The levels of plasma glycolate observed at all doses were significantly higher than those observed with lumasiran at 3 mg/kg or 6 mg/kg.

Discussion: This study represents the first clinical experience with Compound 1a, a novel, orally available, potent, and highly selective glycolate oxidase inhibitor. Oral administration of single doses of Compound 1a up to 3,000 mg and multiple doses (to steady state) up to 2,000 mg per day (1,000 mg qd) were well tolerated and not associated with clinical or laboratory safety signals of potential clinical concern in healthy adult volunteers. Adverse events were mostly mild and no more than moderate in intensity. The lack of any signals of hepatic stress or injury, electrolyte disturbances, adverse hematological effects, or electrocardiographic changes associated with Compound 1a administration support further development.

Plasma glycolate has been used as a measure of target engagement associated with the level of inhibition of GO in both nonclinical studies and clinical studies in healthy adult volunteers. In non-human primates (NHPs), the level of plasma glycolate was shown to increase in a nonlinear manner with the extent of GO mRNA knockdown by RNA interference, increasing rapidly at reductions in GO protein expression greater than 75%. Similar relationships between reductions in Hao1 mRNA expression and increases in serum glycolate levels were observed in rodents treated with lumasiran, with greater suppression of HOAX1 leading to greater reductions in excreted oxalate.

Plasma glycolate was used as a pharmacodynamic measure of target engagement in healthy adult volunteers receiving single doses of lumasiran. After receiving a single subcutaneous dose of lumasiran of 0.3, 1.0, 3.0, or 6.0 mg/kg, plasma glycolate increased to a measured peak of ˜1.6, ˜2.5, ˜3.1, and ˜7.3-fold relative to baseline levels. Notably, the 3.0 mg/kg dose is the approved dosage used in patients >20 kg despite greater elevations in plasma glycolate observed at the 6.0 mg/kg dose.

In this Phase 1 study, Compound 1a achieved mean maximal increases of plasma glycolate concentration in healthy volunteers of ˜4-fold to ˜10-fold, relative to baseline after single doses of 40 to 3,000 mg. Above single doses of 1,000 mg, mean plasma glycolate concentrations plateau through 24 hours, suggesting Compound 1a is maximally inhibiting GO through the dosing period. The increases in plasma glycolate observed with Compound 1a at all doses are substantially higher than the ˜3-fold increase observed in healthy volunteers after a single 3 mg/kg dose of lumasiran, the approved dose used in patients >20 kg. At steady state, Compound 1a results in plasma glycolate concentrations comparable to those measured in case reports of individuals with germline HAO1 knockout, the gene encoding GO, suggesting complete inhibition.

By maximally inhibiting GO, Compound 1a has the potential to lower oxalate production to a greater extent and enable a greater proportion of patients to achieve normalization of urinary oxalate excretion than other treatments.

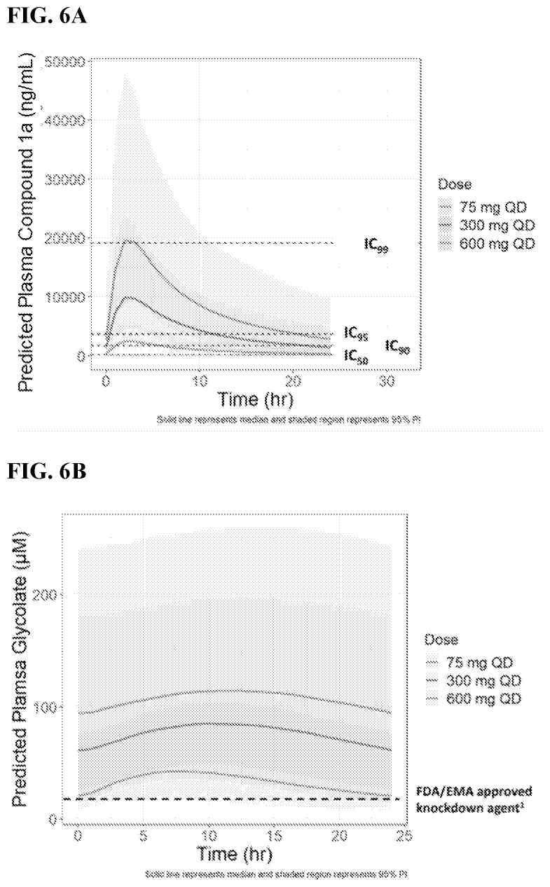

Conclusion: Compound 1a administration was well tolerated up to evaluated single doses of 3,000 mg and multiple doses of 1,000 mg, not associated with safety signals of potential clinical concern, and resulted in the greatest increases in plasma glycolate concentrations of all GO-targeting agents that have been tested in healthy adult volunteers. This study provides dosing and safety information for Compound 1a in patients with PH1 and recurrent stone formers with hyperoxaluria due to hepatic overproduction of oxalate. Pharmacokinetic data supports once daily administration of Compound 1a and rapid onset of action. Pharmacodynamic data (plasma glycolate) suggests Compound 1a rapidly and maximally inhibits GO. Pharmacokinetic-pharmacodynamic (PK/PD) modeling suggests Compound 1a inhibits GO up to 97%. Based on these results, PK/PD modeling was performed to select doses to target the IC50, IC90, and IC95 values for Compound 1a in a Phase 2/3 study (FIGS. 6A-6B). Details of the study are presented in Example 2.

### Example 2: A Two-Part Multinational, Randomized, Placebo-Controlled Trial to Evaluate the Safety and Efficacy of Compound 1a for the Treatment of Children (≥6 Years of Age) and Adults with Primary Hyperoxaluria Type 1 (a Phase 2/3 Clinical Study—Study Synopsis)

A Phase 2/3 Clinical Study is performed to investigate the safety and efficacy of Compound 1a for the treatment of children and adults with primary hyperoxularia type 1 (PH1). Subjects of the study generally have intact renal function. Key inclusion criteria will include age ≥6 years, confirmation of AGXT gene mutation, uOxalate excretion of >0.7 mmol/1.73 m² per 24 hours, eGFR≥30 mL/min/1.73 m², pOxalate <20 μmol/L, and, for subjects taking pyridoxine, stability for 3 months prior to screening. Key exclusion criteria will include prior receipt of a liver transplant, receipt of renal replacement therapy, treatment with lumasiran within 9 months of screening, treatment with experimental RNAi agents (e.g., nedosiran) within 9 months of screening, and female participants that are pregnant, planning to become pregnant, or breastfeeding. Follow on studies may evaluate younger patients and those with impaired renal function. Details of the study described herein are tentative and subject to change.

Part A is a single-blind, placebo-controlled, sequential group design to select a dose of Compound 1a that exhibits maximal urinary oxalate (uOxalate) reduction and an acceptable safety profile in pediatric (≥6 years of age) and adult participants with Primary Hyperoxaluria Type 1 (PH1).

Part B is a parallel group, placebo-controlled study to establish the efficacy and safety of Compound 1a in pediatric (≥6 years of age) and adult participants with PH1.

### 2.1 Objectives, Endpoints, and Estimands:

| Objectives | Endpoints |
|---|---|
| Primary | |
| Part A: | Part A: |
| To select a safe dose of Compound 1a for | Adverse event (AE)/serious adverse event (SAE) |
| administration in Part B. | incidence and severity, clinical chemistry and |
| | hematology laboratory data, electrocardiograms |
| | (ECGs), and vital signs |
| | Percent change from baseline to the end of |
| | treatment period in 24-hour uOxalate excretion |
| Part B - Treatment Period: | Part B - Treatment Period: |
| To establish the efficacy of Compound 1a for | Proportion of participants with response in |
| the treatment of PH1. | 24-hour uOxalate reduction at the end of the |
| | open-label treatment period. Response is defined |
| | as having achieved normal 24-hour excretion |
| | (<0.46 mmol/1.73 m²) or a ≥50% reduction in |
| | 24-hour uOxalate excretion from baseline. |
| Part B - Randomized Withdrawal Period | Part B - Randomized Withdrawal Period: |
| (optional): | Proportion of participants with maintenance of |
| To establish the efficacy of Compound 1a for | response upon Compound 1a withdrawal after |
| the treatment of PH1. | completion of the double-blind, randomized |
| | withdrawal period. |
| Secondary | |
| Part A: | Proportion of participants that achieve normal |
| To support the efficacy of Compound 1a for | 24-hour uOxalate excretion (<0.46 |
| the treatment of PH1 | mmol/1.73 m²) at the end of treatment period |
| To characterize pharmacokinetic parameters | Proportion of participants that achieve a ≥50% |
| in the participants with PH1 | reduction in 24-hour uOxalate from baseline to |
| | the end of the treatment period |
| | Proportion of participants that achieve normal |
| | uOxalate:urine creatinine (uCreatinine) ratio |
| | (normal <63.7 mg/mg [80 mmol/mmol]) |
| | Change (absolute and percent) over time in |
| | 24-hour uOxalate excretion |
| | Change (absolute and percent) in plasma |
| | oxalate (pOxalate) concentration over time |
| | Pharmacokinetics of Compound 1a (steady- |
| | state at Day 28) |
| Part B: | Proportion of participants that achieve normal |
| To support the efficacy and safety of | 24-hour uOxalate excretion (<0.46 |
| Compound 1a for the treatment of PH1 | mmol/1.73 m²) at the end of treatment period |
| | Proportion of participants that achieve a ≥50% |
| | reduction in 24-hour uOxalate from baseline to |
| | the end of the treatment period |
| | Proportion of participants that achieve normal |
| | uOxalate:uCreatinine ratio (normal <63.7 |
| | mg/mg [80 mmol/mmol]) |
| | Change (absolute and percent) over time in |
| | 24-hour uOxalate excretion |
| | Change (absolute and percent) in plasma |
| | oxalate (pOxalate) concentration over time |
| | Change (absolute and percent) from |
| | randomization to end of randomized |
| | withdrawal period in 24-hour uOxalate |
| | excretion |
| | Change (absolute and percent) from |
| | randomization to end of randomized |
| | withdrawal period in plasma oxalate (pOxalate) |
| | concentration over time |
| | AE/SAE incidence and severity, clinical |
| | chemistry and hematology laboratory data, |
| | ECGs, and vital signs |
| Exploratory | |
| Parts A and B: | Plasma glycolate (pGlycolate) concentration |
| To understand the efficacy of Compound 1a | over time |
| on longer-term clinical consequences of | Urinary glycolate (uGlycolate):uCreatinine ratio |
| hyperoxaluria in participants with PH1 | over time |
| | Change from baseline in pGlycolate over time |
| | Change in uGlycolate:uCreatinine ratio over |
| | time |
| | Change in estimated glomerular filtration rate |
| | (eGFR) over time |
| | Incidence of new kidney stones |
| | Change from baseline to end of randomized |
| | withdrawal period in nephrocalcinosis score |
| | based on radiologic assessment of kidney |
| | ultrasound procedure by adjudication/central |
| | reader |
| | Change in quality of life using the Kidney |
| | Disease Quality of Life Questionnaire (KDQOL) |
| | Instrument and the Pediatric Quality of Life |
| | Inventory (PedsQL [generic and end-stage renal |
| | disease [ESRD]) for participants <18 years of |
| | age; and the EQ-5D (EQ-5D-5L for adults and |
| | EQ-5D-Y for participants <18 years of age). |

### 2.2 Overall Design

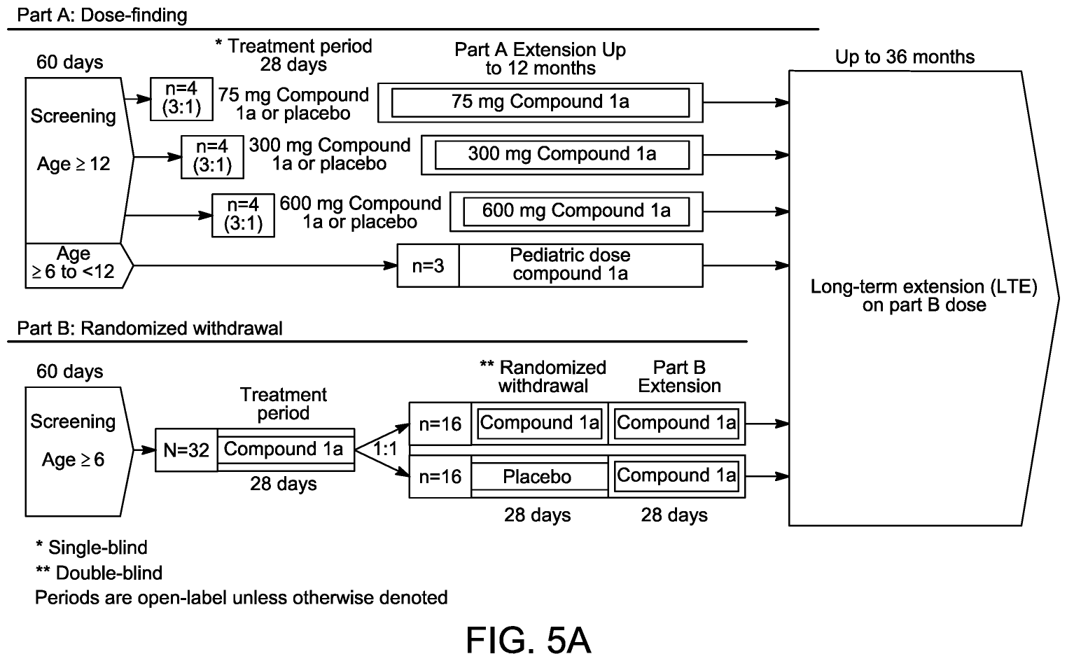

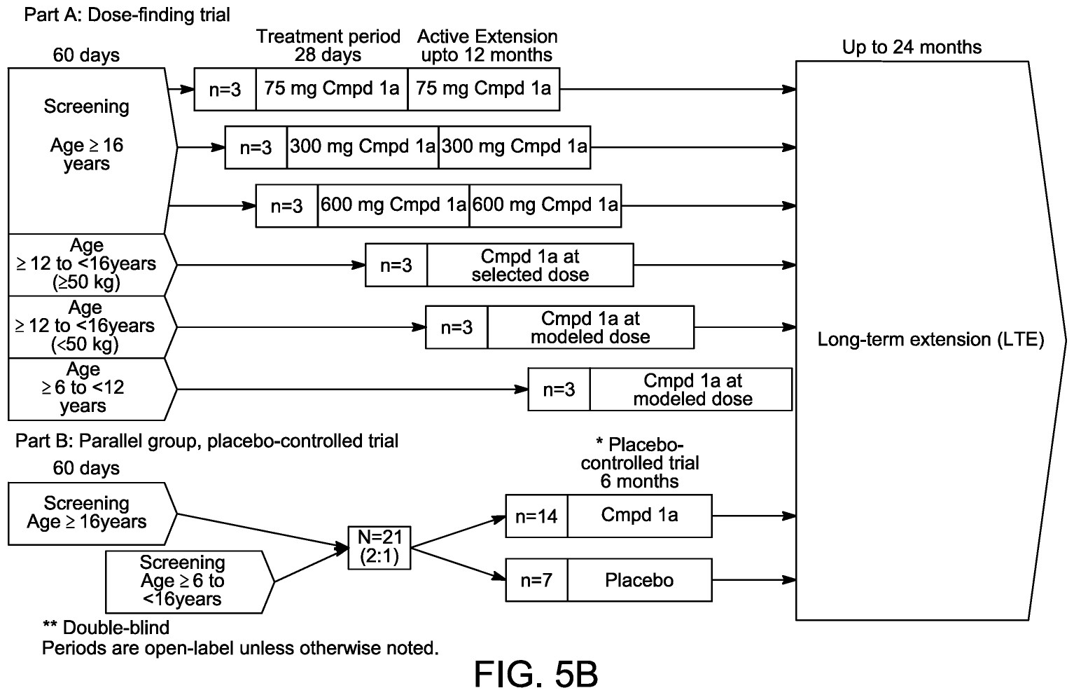

This is an operationally seamless, two-part, Phase 2/3 study. FIG. 5B schematically illustrates details of the study.

Part A is a randomized, single-blind, placebo-controlled, sequential group design evaluating multiple doses of Compound 1a and will consist of two periods:
a 28-day single-blind treatment period, and
an open-label, active extension period (called Part A Extension) lasting up to 12 months

Following a 60-day screening period, eligible adult (≥18 years old) and adolescent participants (≥16 years old) will be allowed to enroll in Part A. Enrollment will start with Group A at 75 mg once a day (QD), wherein 4 participants will be randomized 3:1 active to placebo. After 4 participants have been randomized into Group A, Group B will be immediately open for enrollment at 300 mg QD, followed by Group C at 600 mg QD with each group randomized 3:1 active to placebo. After each dose group has enrolled 4 participants, additional participants may be recruited into dose groups if additional pharmacokinetic (PK) or pharmacodynamic (PD) data are required to select the dose for Part B.

After the last participant in Group C has completed the Part A treatment period, an independent Data Monitoring Committee (DMC) will review all data for the three dose groups from the Part A treatment period to select a dose for Part B.

Based on the selected Part B dose, pharmacokinetic/pharmacodynamic (PK/PD) data will be used to estimate a weight-dependent dose by PK/PD modeling and simulation for pediatric participants (≥12 to <16 years and ≥50 kg). Three pediatric participants will then be enrolled in a separate pediatric dose group and receive Compound 1a (no randomization to placebo). After the pediatric dose group has enrolled 3 participants, additional participants of lower weight and/or age (e.g., (i) ≥12 to <16 years and <50 kg or (ii) ≥6 to <12 years old) may be enrolled at modeled doses.

After completion of the Part A single-blind treatment period, adult, adolescent, and pediatric participants may continue to receive Compound 1a during the Part A Extension lasting up to 24 months. Participants who were randomized to placebo will be converted in a blinded manner to Compound 1a at the dose administered in their respective treatment groups. Treatment assignments during the single-blind treatment period will not be revealed to the participants. Study participants will remain in the Part A Extension until the Part B dose is selected.

Once sufficient data are available to support selection of the Part B dose, the Part A Extension will be closed, and all adult and adolescent participants in Part A Extension will have the option to continue in the optional long-term extension (LTE) and convert to the selected Part B dose. Pediatric participants (≥6 to <12 years old or <50 kg) will have the option to directly enter the LTE period after completion of the Part A treatment period and continue to receive their pediatric dose.

Part B is a pivotal, parallel group, placebo-controlled design consisting of a 6-month placebo-controlled trial and a subsequent long-term extension period of up to 24 months.

Part B will initiate with a 60-day screening period for adults and adolescents (≥16 years old) and pediatric patients (6 to <16 years old). Enrollment of pediatric patients may be dependent upon data from the Part A pediatric dose group.

Following the screening period, eligible participants (N=21 with 2:1 acive:placebo) will enter the treatment period at the dose of Compound 1a selected based on Part A data (ie, the Part B dose).

At the end of the treatment period, participants will have the option to continue in the LTE.

### 2.3 Number of Participants:

The target accrual for Part A is a minimum of 12 participants at three different dose levels (9 participants on Compound 1a, 3 participants on placebo). Part B will enroll approximately 21 participants into the Compound 1a treatment phase to provide primary efficacy data for data analysis in the RWP of Part B.

Note: Enrolled means a participant's, or their legally authorized representative's, agreement to participate in a clinical study following completion of the informed consent process and screening. Potential participants who are screened for the purpose of determining eligibility for the study, but do not participate in the study, are not considered enrolled, unless otherwise specified by the protocol. A participant will be considered enrolled if the informed consent is not withdrawn prior to participating in any study activity after screening.

### 2.4 Intervention Groups and Duration:

Part A will consist of at least four groups:
Group A at 75 mg QD;
Group B at 300 mg QD;
Group C at 600 mg QD; and
Pediatric dose groups: Group D (≥12 to <16 years old and ≥50 kg), Group E ((≥12 to <16 years old and <50 kg), and Group F (≥6 to <12 years old) (doses for Groups D-F to be determined from PK-PD modeling data).

The total duration from Screening through the end of Part A is up to approximately 15 months, with each participant's time in the Part A Extension dependent on timing of enrollment.

Part B will consist of 2 arms of participants randomized (2:1) to either Compound 1a or matching placebo. The total duration from Screening through the end of Part B treatment period is approximately 240 days.

Participants who complete Part A or Part B will have the option to enroll in the 24-month long-term extension period.

### 2.5 Inclusion Criteria:

Eligible participants must have signed and dated an Institutional Review Board (IRB)/Independent Ethics Committee (IEC)-approved Informed Consent Form (ICF) before screening procedures start. Participants must meet the following criteria for inclusion during screening:

### Age and Weight

1. Participants must be ≥6 or ≥12 years of age at the time of signing the informed consent or assent, depending on the progress of the study
   a. Part A Groups A, B, and C participants must be adults or adolescents (≥16 years old).
   b. Part A pediatric dose group participants in Group D (≥12 to <16 years old and ≥50 kg), Group E ((12 to <16 years old and <50 kg), and Group F (≥6 to <12 years old) may enroll once appropriate pediatric dose(s) has been estimated from PK/PD modeling and simulation.
   c. Part B participants must be adults or adolescents (16 years old) until the Part A pediatric dose group data are available to support the use of the pediatric dose in participants ≥6 to <16 years old.
   d. Part B pediatric participants (6 to <16 years old) may enroll once the Part A pediatric dose group data are available to support the use of the pediatric dose in additional participants.

### Type of Participant and Disease Characteristics

1. Participant must have a documented diagnosis of PH1 with an alanine-glyoxylate aminotransferase gene (AGXT) gene mutation
   Note: A genetic analysis report for AGXT will be acceptable. In the absence of any documentation for the AGXT mutation, participants must be willing to undergo genetic analysis. Participants who have undergone an allogeneic bone marrow transplant must provide AGXT variant analysis report performed prior to the bone marrow transplant to be eligible for the study. Participants who have received a blood transfusion must wait 2 weeks following the blood transfusion before undergoing genetic testing.
2. uOxalate excretion: >0.7 mmol/1.73 m² per 24 hours
3. eGFR ≥30 mL/min/1.73 m² eGFR should be estimated using Chronic Kidney Disease Epidemiology Collaboration refit without the race variable equation for participants age ≥18 and the Bedside Schwartz equation for participants age ≥6, but <18
4. pOxalate <20 μmol/L
5. If taking pyridoxine (Vitamin B6), participants must be on a stable dose for 3 months prior to screening and willing to stay on this dose for the duration of the study, and their plasma pyridoxine level must be within the normal range at Screening.

### Sex and Contraceptive/Barrier Requirements

1. Male participants must use a double-barrier method (eg, condoms with spermicidal gel or foam) during vaginal intercourse and should not father a child nor donate sperm while taking Compound 1a and for 30 days after the last dose of Compound 1a. Condoms are not required if the participant is vasectomized or if the participant's partner is not a female of childbearing potential
2. Postmenopausal females and females not of childbearing potential may participate in this study without the use of contraception:
   a. A postmenopausal state is defined as no menses for 12 months without an alternative medical cause and must be confirmed at Screening visit with plasma follicle-stimulating hormone (FSH)
   b. Females are considered not of childbearing potential if they have had surgical bilateral oophorectomy (with or without hysterectomy) at least 6 weeks prior to the start of the study and must be confirmed at Screening visit with plasma FSH.
3. Females of childbearing potential, defined as all females physiologically capable of becoming pregnant, who engage in heterosexual intercourse must use two highly effective methods of contraception starting at Screening and for 30 days following the last dose of Compound 1a.

Contraceptive use by males and females should be consistent with local regulations regarding the methods of contraception for those participating in clinical studies.

### Informed Consent

1. Capable of giving signed informed consent or assent, which includes compliance with the requirements and restrictions listed in the ICF and in this protocol.

### 2.6 Exclusion Criteria:

Participants are excluded from the study if any of the following criteria apply:

### Medical Conditions

1. Participants who are recipients of a liver transplant.

### Prior/Concomitant Therapy

1. Participants who are receiving renal replacement therapy (hemodialysis, peritoneal dialysis, or continuous renal replacement therapies).
2. Participants treated with an approved ribonucleic acid interference (RNAi) agent (eg, lumasiran/Oxlumo®) within 9 months of screening.

### Prior/Concurrent Clinical Study Experience

1. Participants who received any investigational medicinal product within 30 days or 5 half-lives, whichever is longer, prior to the first day on study, or are in follow-up for another interventional clinical study during Screening.
2. Participants treated with an experimental RNAi agent (eg, nedosiran) within 9 months of screening.

### Other Exclusions

1. Female participants who are pregnant, planning a pregnancy, or breastfeeding.
   2.7 Statistical methods:
Part A: Data will be summarized descriptively.
Part B—Treatment Period: The two-sided 95% exact confidence interval for the observed proportion of participants with response in 24-hour uOxalate reduction at the end of the Compound 1a treatment period will be calculated.

### 2.8 Sample Size Determination:

The sample size in Part A is determined on the basis of feasibility considerations, including the rarity of the disease. The target accrual for Part A is a minimum of 12 adult and adolescent participants at three different dose levels (9 participants on Compound 1a, 3 participants on placebo) and a minimum of 3 participants under 16 or 12 years old on Compound 1a at pediatric dose level(s).

Part B will enroll approximately 21 participants into the Compound 1a treatment period.

The primary endpoint of the Compound 1a treatment period in Part B will be evaluated as the proportion of participants with the response in 24-hour uOxalate reduction at the end of the Compound 1a treatment period. For a sample size of 32, the lower bound of the exact 95% confidence interval for the observed proportion of 50% is 32%.

### 2.9 Independent Data Monitoring Committee:

An independent Data Monitoring Committee (DMC) will review all data for the three dose groups from the Part A treatment period prior to dose selection for Part B. Additional data from the Part A Extension may also be reviewed if available. The DMC is a group of independent physicians and scientists who are appointed to monitor the safety and scientific integrity of a human research intervention and to make recommendations to the Sponsor regarding the stopping of a study for efficacy, for harms, or for futility. The composition of the committee is dependent upon the expertise and knowledge required for monitoring this study.

### 2.10 Study Schemes

Study Schemes (Part A and Part B) are shown in FIG. 5B. Details of the study are subject to change and optimization.

### 2.11 Schedule of Assessments

Sample screening schedules for Part A and/or Part B of the study are listed in Table 4, Table 5, and Table 6, each of which is used for study assessments detailed in the clinical protocol.

TABLE 4
Sample Screening Schedule for Part A or Part B

| | Screening |
|---|---|
| Visit type ¹ | CV |
| Visit Day (±Window) | Day −60 to Day −1 |
| Informed consent | X |
| Demographics | X |
| Medical history, including kidney stone history | X |
| Prior/concomitant medications/procedures | X |
| Physical examination ² | X |
| Vital signs ³ | X |
| 12-lead ECG ⁴ | X |
| Blood Collections | |
| Safety laboratory tests ⁵ | X |
| Pyridoxine (if taking pyridoxine) ⁶ | (X) |
| Serum virology ⁷ | X |
| Genetic analysis ⁸ | X |
| FSH (postmenopausal women) | (X) |
| Optional exploratory research samples ⁹ | X |
| Urine Collections | |
| Complete urinalysis ¹⁰ | X |
| Urine pregnancy test (WOCBP) ¹¹ | (X) |
| 24-hour urine for PD analysis ¹² | X |
| Healthcare resource utilization | X |
| Adverse events ¹³ | X |
| Contraception reminder | X |
| Dietary reminder | X |

Abbreviations: ALT = alanine aminotransferase; ALP = alkaline phosphatase; AST = aspartate aminotransferase; AXGT = Alanine-glyoxylate aminotransferase (gene name); BUN = blood urea nitrogen; CV = clinic visit; ECG = electrocardiogram; eGFR = estimated glomerular filtration rate; FSH = follicle-stimulating hormone; HBsAg = hepatitis B virus surface antigen; Hct = hematocrit; HCV = hepatitis C virus; Hgb = hemoglobin; HIV = human immunodeficiency virus; INR = international normalized ratio; PD = pharmacodynamic; PT = thromboplastin time; PTT = partial thromboplastin time; RBC = red blood cells; SARS-CoV-2 = severe acute respiratory syndrome coronavirus 2; SGOT = serum glutamic-oxaloacetic transaminase; SGPT = serum glutamic pyruvic transaminase; WBC = white blood cell; WOCBP = women of childbearing potential.
¹ The Screening visit must be performed in the clinic. Sample collection of genetic analysis may be done via a home health visit after participant has given informed consent.
² A complete physical examination will be performed at Screening and will include height and weight.
³ Vital signs will be measured after 5 minutes rest and will include supine or sitting systolic and diastolic blood pressure, heart rate, respiratory rate, and temperature.
⁴ When ECG and blood sample collection occur at the same time, ECG should be performed before blood samples are drawn, when possible.
⁵ Safety laboratory tests include chemistry (albumin; ALT/SGPT; ALP; AST/SGOT; bilirubin, total; BUN; calcium; carbon dioxide, total; chloride; creatinine; eGFR; glucose, non-fasting; potassium; protein, total; sodium), hematology (RBC, Hgb, Hct, RBC indices, WBC with differentials), and coagulation (PT/PTT/INR).
⁶ Only participants who are taking pyridoxine (Vitamin B6) are required to have a blood sample collected at least 6 hours after their last dose of pyridoxine. Participants should be instructed to take their pyridoxine after blood collection if possible.
⁷ Serologic testing for HIV, Hepatitis B (HbsAg), Hepatitis C (anti-HCV antibody), and polymerase chain reaction for SARS-CoV-2 (or the most appropriate diagnostic test available as the pandemic evolves) will be done at Screening.
⁸ Genetic testing for PHI will be performed (sample may be collected via a home health visit after informed consent) in all participants who cannot provide documentation of prior genetic diagnosis of PHI with a mutation in AGXT. Genetic analysis cannot be performed if a participant has ever had an allogeneic bone marrow transplant or has received a blood transfusion <2 weeks prior to specimen collection.
⁹ If a participant consents, blood will be collected, processed, and stored for future exploratory analyses. If a participant does not consent and provide a sample at Screening, he or she may consent to provide a sample at any time in the study.
¹⁰ Complete urinalysis will be performed (specific gravity, pH, glucose, protein, hemoglobin, leukocyte esterase, nitrite, bilirubin, ketones and occult blood).
¹¹ If the urine pregnancy test is positive, a serum pregnancy test must be performed to confirm the result.
¹² Sample collection will begin after the first morning void and will be collected over 24-hours, including the first void of the next morning. Urine collections will be assessed for validity at the central lab using criteria specified in Section 8.1.1. of the protocol. The 24-hour urine collection should be scheduled as early as possible in the Screening Period to allow time for another collection in the event that the initial collection is deemed invalid, and a repeat collection is required. Details for sample collection and processing are provided in the Laboratory Manual.
¹³ Adverse events will be collected and documented from Screening until the end of the safety reporting period which is 30 days after the final dose of study drug.

TABLE 5
Sample Screening Schedule for Part A

| | Treatment Period | | | | Part A Extension ¹⁸ | |
|---|---|---|---|---|---|---|
| | (Single-blind) | | | | (Open-label) | |
| | Visit Type ¹ | | | | | |
| | CV | HH/TC | HH/TC | CV | HH/TC | CV |
| | Visit Day (±Window) | | | | | |
| | | | | | | Day 84, 140, |
| | | | | | Day 56, 112, | 196, 252, 308, |
| | | Day 7 | Day 14 | Day 28 | 168, 224 | 364 |
| | Day 1 | (±2) | (±2) | (±2) | (±7) | (±7) |
| Physical examination ² | X | | | X | | X |
| Vital signs ³ | X | | | X | X | X |
| 12-lead ECG ⁴ | X | | | X | | |
| Renal ultrasound ⁵ | X | | | | | Day 196 only |
| Blood Collections | | | | | | |
| Safety laboratory tests ⁶ | X | X | X | X | X | X |
| Pyridoxine (if taking | (X) | | | (X) | | (X) |
| pyridoxine) ⁷ | | | | | | |
| PK analysis ⁸ | | | | X | | |
| PD analysis ⁹ | X | | | X | | X |
| Urine Collections | | | | | | |
| Complete urinalysis ¹⁰ | X | | | X | X | X |
| Urine pregnancy test | (X) | | | (X) | (X) | (X) |
| (WOCBP) ¹¹ | | | | | | |
| 24-hour urine for PD | 3X | | | 3X | | X |
| analysis ¹² | | | | | | |
| Random urine for PD | | X | X | | X | |
| analysis ¹³ | | | | | | |
| Participant-Reported | | | | | | |
| Outcomes | | | | | | |
| EQ-5D-5L/EQ-5D-Y ¹⁴ | X | | | X | | Day 196 only |
| KDQOL/PedsQL ¹⁵ | X | | | X | | Day 196 only |
| Patient and caregiver | X | | | X | | Day 196 only |
| survey | | | | | | |
| Healthcare resource | X | X | X | X | X | X |
| utilization collection | | | | | | |
| Kidney stone events | X | X | X | X | X | X |
| Adverse events ¹⁶ | X | X | X | X | X | X |
| Concomitant | X | X | X | X | X | X |
| medications/procedures | | | | | | |
| Contraception reminder | X | X | X | X | X | X |
| Dietary reminder | X | X | X | X | X | X |
| Randomization | X | | | | | |
| Study Drug Administration ¹⁷ | X | X | X | X | X | X |

Abbreviations: ALT = alanine aminotransferase; ALP = alkaline phosphatase; AST = aspartate aminotransferase; BUN = blood urea nitrogen; CV = clinic visit; ECG = electrocardiogram; eGFR = estimated glomerular filtration rate; HH = home health visit; Hct = hematocrit; Hgb = hemoglobin; INR = international normalized ratio; KDQOL = kidney disease quality of life; PD = pharmacodynamic; PedsQL = Pediatric Quality of Life Inventory; PK = pharmacokinetic; PT = thromboplastin time; PTT = partial thromboplastin time; RBC = red blood cell; SGOT = serum glutamic-oxaloacetic transaminase; SGPT = serum glutamic pyruvic transaminase; TC = telephone call; WBC = white blood cell; WOCBP = women of childbearing potential.
¹ Visits designated “CV” must be performed in the clinic. Visits designated “HH” may be done in the clinic or performed via a combination of home health visit and telephone check-in with site staff.
² A complete physical examination will be performed at Day 1 and Day 28 and will include weight, and height will only be measured for participants younger than 18 years of age. A brief physical examination will be performed at all other indicated visits.
³ Vital signs will be measured after 5 minutes rest and will include supine or sitting systolic and diastolic blood pressure, heart rate, respiratory rate, and temperature.
⁴ When ECG and blood sample collection occur at the same time, ECG should be performed before blood samples are drawn, when possible.
⁵ Renal ultrasound for Day 1 may be performed any time in the 14 days preceding and including the Day 1 visit to allow for scheduling flexibility; however, it may only be done after assessment and confirmation of participant eligibility.
⁶ Prior to study drug administration, samples will be collected for safety laboratory tests including chemistry (albumin; ALT/SGPT; ALP; AST/SGOT; bilirubin, total; BUN; calcium; carbon dioxide, total; chloride; creatinine; eGFR calculation; glucose; potassium; protein, total; sodium), hematology (RBC, Hgb, Hct, RBC indices, WBC with differentials), and coagulation (PT/PTT/INR).
⁷ Only participants who are taking pyridoxine (Vitamin B6) are required to have a blood sample collected at least 6 hours after their last dose of pyridoxine. Participants should be instructed to take their pyridoxine after blood collection if possible.
⁸ For purposes of PK sampling on Day 28, participants should administer study drug at the clinic as instructed by research staff. Plasma samples will be collected for PK analysis on Day 28 at pre-dose and post-dose at 2 hours (±15 min), 4 hours (±15 min), and 6 hours (±15 min). It is important to accurately record the following times: the last two administrations of study drug prior to the Day 28 visit, the pre-dose sample collection, the administration of study drug at the Day 28 visit, and the post-dose sample collections.
⁹ For purposes of PD sampling on Day 28, participants should administer study drug at the clinic as instructed by research staff. Plasma samples will be collected for analysis of oxalate and glycolate on Day 28 at pre-dose and post-dose at 2 hours (±15 min), 4 hours (±15 min), and 6 hours (±15 min). At all other visits indicated, a pre-dose sample will be collected.
¹⁰ Complete urinalysis will be performed (specific gravity, pH, glucose, protein, hemoglobin, leukocyte esterase, nitrite, bilirubin, ketones and occult blood).
¹¹ If the urine pregnancy test is positive, a serum pregnancy test must be performed to confirm the result.
¹² Sample collection will begin after the first morning void and will be collected over 24-hours, including the first void of the next morning. For calculation of a Treatment Period Day 1 baseline, three 24-hour urine samples will be collected in the 14 days preceding the Day 1 visit; however, these collections may only be done after assessment and confirmation of participant eligibility. For calculation of the primary endpoint at Day 28, three 24-hour urine samples will be collected between the Day 14 and Day 28 visits. For visits during the Part A Extension, the 24-hour urine collection should be collected within 7 days prior to the scheduled visit. Urine collections will be assessed for validity at the central lab using criteria specified in Section 8.1.1 of the protocol. If a 24-hour urine collection is deemed invalid, the site will instruct the participant to collect another 24-hour urine sample as soon as possible. Details for sample collection and processing are provided in the Laboratory Manual.
¹³ Single void collections for random urine sample for PD analysis must be collected as a first morning void and prior to study drug administration.
¹⁴ In regions where the scale is available, participants 18 years of age and older will be administered the EQ-5D-5L, and participants younger than 18 years of age will be administered the EQ-5D-Y.
¹⁵ In regions where the scale is available, participants 18 years of age and older will be administered the KDQOL, and participants younger than 18 years of age will be administered the PedsQL.
¹⁶ Adverse events will be collected and documented until the end of the safety reporting period which is 30 days after the final dose of study drug.
¹⁷ On Day 1, study drug will be administered in clinic after all other study assessments are completed. On Day 28, study drug will be administered in clinic after the pre-dose plasma PK sample has been collected.
¹⁸ Except for the pediatric dose group participants, all participants will continue in the Part A Extension until a Part B dose is selected, at which point they will have the option to enter the long-term extension period. Pediatric dose group participants will not participate in the Part A Extension and will instead have the option to directly enter the long-term extension period.

TABLE 6
Sample Screening Period for Part B

| | Treatment Period | | | | Part B Extension | | |
|---|---|---|---|---|---|---|---|
| | (Open-label) | | | | (Open-label) | | |
| | Visit Type ¹ | | | | | | |
| | CV | HH/TC | HH/TC | CV | HH/TC | HH/TC | CV |
| | Visit Day (±Window) | | | | | | |
| | | Day 7 | Day 14 | Day 28 | Day 7 | Day 14 | Day 28 |
| | Day 1 | (±2) | (±2) | (±2) | (±2) | (±2) | (±2) |
| Physical examination ² | X | | | X | | | X |
| Vital signs ³ | X | | | X | | | X |
| 12-lead ECG ⁴ | X | | | X | | | X |
| Renal ultrasound ⁵ | X | | | | | | X |
| Blood Collections | | | | | | | |
| Safety laboratory tests ⁶ | X | | | X | | | X |
| Pyridoxine (if taking | (X) | | | (X) | | | (X) |
| pyridoxine) ⁷ | | | | | | | |
| PK analysis ⁸ | | | | X | | | |
| PD analysis ⁹ | X | X | X | X | X | X | X |
| Urine Collections | | | | | | | |
| Complete urinalysis ¹⁰ | X | | | X | | | X |
| Urine pregnancy test | (X) | | | (X) | | | (X) |
| (WOCBP) ¹¹ | | | | | | | |
| 24-hour urine for PD | 3X | | | 3X | | | X |
| analysis ¹² | | | | | | | |
| Random urine for PD | | X | X | | X | X | |
| analysis ¹³ | | | | | | | |
| Participant-Reported | | | | | | | |
| Outcomes | | | | | | | |
| EQ-5D-5L/EQ-5D-Y ¹⁴ | X | | | | | | |
| KDQOL/PedsQL ¹⁵ | X | | | | | | |
| Patient and caregiver | X | | | | | | |
| survey | | | | | | | |
| Healthcare resource | X | X | X | X | X | X | X |
| utilization | | | | | | | |
| Kidney stone events | X | X | X | X | X | X | X |
| Adverse events ¹⁶ | X | X | X | X | X | X | X |
| Concomitant | X | X | X | X | X | X | X |
| medications/procedures | | | | | | | |
| Contraception reminder | X | X | X | X | X | X | X |
| Dietary reminder | X | X | X | X | X | X | X |
| Randomization | | | | X | | | |
| Study Drug Administration | X | X | X | X | X | X | X |

Abbreviations: ALT = alanine aminotransferase; ALP = alkaline phosphatase; AST = aspartate aminotransferase; BUN = blood urea nitrogen; CV = clinic visit; ECG = electrocardiogram; eGFR = estimated glomerular filtration rate; HH = home health visit; Hct = hematocrit; Hgb = hemoglobin; INR = international normalized ratio; KDQOL = kidney disease quality of life; PD = pharmacodynamic; PedsQL = Pediatric Quality of Life Inventory; PK = pharmacokinetic; PT = thromboplastin time; PTT = partial thromboplastin time; RBC = red blood cell; RWP = randomized withdrawal period; SGOT = serum glutamic-oxaloacetic transaminase; SGPT = serum glutamic pyruvic transaminase; TC = telephone call; WBC = white blood cell; WOCBP = women of childbearing potential.
¹ Visits designated “CV” must be performed in the clinic. Visits designated “HH” may be done in the clinic or performed via a combination of home health visit and telephone check-in with site staff.
² A complete physical examination will be performed at Treatment Period Day 1 and RWP Day 28 and will include weight, and height will only be measured for participants younger than 18 years of age. A brief physical examination will be performed at all other indicated visits.
³ Vital signs will be measured after 5 minutes rest and will include supine or sitting systolic and diastolic blood pressure, heart rate, respiratory rate, and temperature.
⁴ When ECG and blood sample collection occur at the same time, ECG should be performed before blood samples are drawn, when possible.
⁵ Renal ultrasound for Day 1 may be performed any time in the 14 days preceding and including the Day 1 visit to allow for scheduling flexibility; however, it should be done after assessment and confirmation of participant eligibility.
⁶ Prior to study drug administration, samples will be collected for safety laboratory tests including chemistry (albumin; ALT/SGPT; ALP; AST/SGOT; bilirubin, total; BUN; calcium; carbon dioxide, total; chloride; creatinine; eGFR calculation; glucose; potassium; protein, total; sodium), hematology (RBC, Hgb, Hct, RBC indices, WBC with differentials), and coagulation (PT/PTT/INR).
⁷ Only participants who are taking pyridoxine (Vitamin B6) are required to have a blood sample collected at least 6 hours after their last dose of pyridoxine. Participants should be instructed to take their pyridoxine after blood collection if possible.
⁸ On the days of PK sampling, participants should administer study drug at the clinic as instructed by research staff. Population plasma PK samples will be collected pre-dose and 2 hours (±15 min) post-dose on both Treatment Period Day 28 and RWP Day 28. On each of these days, it is important to accurately record the following times: the last two administrations of study drug prior to the visit, the pre-dose sample collection, the administration of study drug at the visit, and the post-dose sample collection.
⁹ Plasma samples will be collected for analysis of oxalate and glycolate pre-dose on the days specified.
¹⁰ Complete urinalysis will be performed (specific gravity, pH, glucose, protein, hemoglobin, leukocyte esterase, nitrite, bilirubin, ketones and occult blood).
¹¹ If the urine pregnancy test is positive, a serum pregnancy test must be performed to confirm the result.
¹² Sample collection will begin after the first morning void and will be collected over 24-hours, including the first void of the next morning. For calculation of a Treatment Period Day 1 baseline, three 24-hour urine samples will be collected in the 14 days preceding the Day 1 visit; however, these collections should be done after assessment and confirmation of participant eligibility. For calculation of the primary endpoints at Treatment Period Day 28 and RWP Day 28, three 24-hour urine samples will be collected between the Day 14 and Day 28 visits of each respective period. For the Part B Extension Day 28 visit, the 24-hour urine collection should be collected within 3 days prior to the scheduled visit. Urine collections will be assessed for validity at the central lab using criteria specified in Section 8.1.1 of the protocol. If a 24-hour urine collection is deemed invalid, the site will instruct the participant to collect another 24-hour urine sample as soon as possible. Details for sample collection and processing are provided in the Laboratory Manual.
¹³ Single void collections for random urine sample for PD analysis should be collected as a first morning void and prior to study drug administration.
¹⁴ In regions where the scale is available, participants 18 years of age and older will be administered the EQ-5D-5L, and participants younger than 18 years of age will be administered the EQ-5D-Y.
¹⁵ In regions where the scale is available, participants 18 years of age and older will be administered the KDQOL, and participants younger than 18 years of age will be administered the PedsQL.
¹⁶ Adverse events will be collected from Screening and documented until the end of the safety reporting period which is 30 days after the final dose of study drug

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of treating hyperoxaluria in a subject in need thereof, comprising administering to the subject a therapeutically effective amount of Compound 1, having the formula: 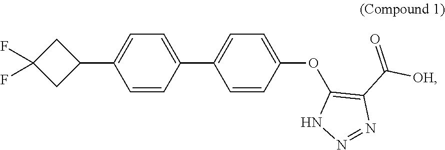 [SMILES: O=C(O)c1nn[nH]c1Oc1ccc(-c2ccc(C3CC(F)(F)C3)cc2)cc1]
a tautomer, a pharmaceutically acceptable salt, a hydrate, a solvate, or a combination thereof, wherein the therapeutically effective amount reduces a maximal urinary oxalate (uOxalate) excretion: i) to less than about 0.46 millimoles per 1.73 square meters per 24 hours (mmol/1.73 m²/24 hr)), and/or ii) by at least about 50% relative to a uOxalate excretion prior to the treating.

2. The method of claim 1, wherein the therapeutically effective amount is a total daily dosage of from about 5 mg to about 3,000 mg.

3. A method of treating hyperoxaluria in a subject in need thereof, comprising administering to the subject a therapeutically effective amount of Compound 1, having the formula: 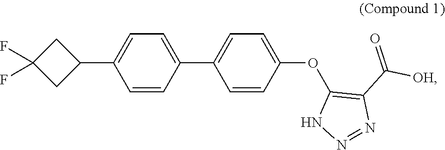 [SMILES: O=C(O)c1nn[nH]c1Oc1ccc(-c2ccc(C3CC(F)(F)C3)cc2)cc1]
a tautomer, a pharmaceutically acceptable salt, a hydrate, a solvate, or a combination thereof, wherein the therapeutically effective amount is a total daily dosage of from about 20 milligrams (mg) to about 3,500 mg.

4. The method of claim 3, wherein the therapeutically effective amount is a total daily dosage of about 20 mg to 2,500 mg.

5. A method of preventing, delaying the onset of, ameliorating, preventing the progression of, alleviating, reducing, improving, eliminating, or curing a symptom and/or complication of hyperoxaluria in a subject in need thereof, comprising administering to the subject a therapeutically effective amount of Compound 1, having the formula: 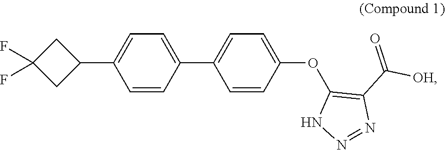 [SMILES: O=C(O)c1nn[nH]c1Oc1ccc(-c2ccc(C3CC(F)(F)C3)cc2)cc1]
a tautomer, a pharmaceutically acceptable salt, a hydrate, a solvate, or a combination thereof, wherein the therapeutically effective amount is a total daily dosage of from about 20 milligrams (mg) to about 3,500 mg.

6. The method of any one of claims 1 to 5, wherein the hyperoxaluria is a primary hyperoxaluria.

7. The method of claim 6, wherein the primary hyperoxaluria is primary hyperoxaluria type 1 (PH1).

8. The method of any one of claims 5 to 7, wherein the symptom and/or complication of hyperoxaluria, primary hyperoxaluria, or PH1 is urolithiasis, nephrolithiasis, nephrocalcinosis, recurrent stone formation, chronic kidney disease, kidney failure, end stage renal disease, failure to gain weight, failure to thrive, lethargy, tiredness, loss of appetite, nausea, vomiting, swelling of hands and feet, pale skin color (e.g., relating to anemia), calcium oxalate deposits in kidneys, stones in bladder or urethra, hematuria, dysuria, reduced urine output, urge to urinate often, abdominal pain, renal colic, blockage of urinary tract, repeated urinary tract infections, bedwetting (enuresis), difficulty controlling urine, systemic oxalosis (oxalate accumulation in various organ systems including bone, skin, retinas, myocardium, blood vessels, and/or central nervous system), bone pain, bone fractures, osteosclerosis, abnormal hardening and density of bone, erythropoietin-resistant anemia, optic atrophy, retinopathy, dental pain, tooth mobility, pulp exposure, root resorption, peripheral neuropathy, heart block, arrhythmias, myocarditis, cardioembolic stroke, vasospasm, arthropathy, hepatosplenomegaly, livedo reticularis, necrosis of hands and feet (peripheral gangrene), or calcinosis cutis metastatica.

9. The method of any one of claims 1 to 8, wherein the hyperoxaluria is caused by overproduction of oxalate.

10. A method of lowering a urinary oxalate level in a subject, comprising administering to the subject in need thereof a therapeutically effective amount of Compound 1, having the formula: 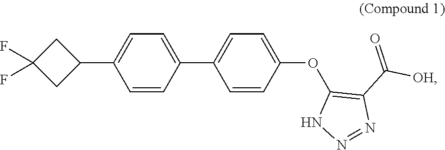 [SMILES: O=C(O)c1nn[nH]c1Oc1ccc(-c2ccc(C3CC(F)(F)C3)cc2)cc1]
a tautomer, a pharmaceutically acceptable salt, a hydrate, a solvate, or a combination thereof, wherein the therapeutically effective amount is a total daily dosage of about 20 milligrams (mg) to about 3,500 mg.

11. A method of lowering a plasma oxalate level in a subject, comprising administering to the subject in need thereof a therapeutically effective amount of Compound 1, having the formula: 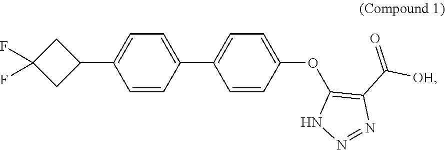 [SMILES: O=C(O)c1nn[nH]c1Oc1ccc(-c2ccc(C3CC(F)(F)C3)cc2)cc1]
a tautomer, a pharmaceutically acceptable salt, a hydrate, a solvate, or a combination thereof, wherein the therapeutically effective amount is a total daily dosage of about 20 milligrams (mg) to about 3,500 mg.

12. The method of any one of claims 1 to 11, wherein the subject has one or more mutations in an alanine-glyoxylate aminotransferase (AGXT) gene.

13. The method of any one of claims 1 to 12, wherein the subject has a uOxalate excretion of more than about 0.46 mmol/1.73 m² per 24 hours.

14. The method of claim 13, wherein the subject has a uOxalate excretion of more than about 0.7 mmol/1.73 m² per 24 hours.

15. The method of any one of claims 1 to 14, wherein the subject has a plasma glycolate (pGlycolate) concentration of at least about 50 micromoles per liter (μmol/L).

16. The method of claim 15, wherein the subject has a pGlycolate concentration of at least about 20 μmol/L.

17. The method of any one of claims 1 to 16, wherein the subject has a plasma glycolate (pGlycolate) concentration of less than about 20 μmol/L.

18. The method of claim 17, wherein the subject has a pGlycolate concentration of less than about 10 μmol/L.

19. The method of any one of claims 1 to 18, wherein the subject has an estimated glomerular filtration rate (eGFR) of at least about 30 mL/min/1.73 m².

20. The method of claim 19, wherein the subject has an eGFR of at least about 90 mL/min/1.73 m².

21. The method of any one of claims 1 to 20, wherein the subject receives pyridoxine (Vitamin B6) at a stable dose for at least three (3) months prior to the treating and maintains the same dose during the treating.

22. The method of claim 21, wherein the subject has a plasma pyridoxine level within a normal range.

23. The method of any one of claims 1 to 22, wherein:
1) the subject is not a recipient of a liver transplant;
2) the subject does not receive a renal replacement therapy selected from the group consisting of hemodialysis, peritoneal dialysis, a continuous renal replacement therapy, or a combination thereof, and/or
3) the subject is not treated with a ribonucleic acid interference (RNAi) agent for at least nine (9) months prior to the treating.

24. The method of claim 23, wherein the RNAi agent is lumasiran.

25. The method of any one of claims 1 to 24, wherein the subject is human.

26. The method of claim 25, wherein the human is at least 18 years of age.

27. The method of claim 25, wherein the human is an adult of at least 18 years old or an adolescent of at least 12 years old with a body weight of at least 50 kg.

28. The method of claim 25, wherein the human is less than 18 years old.

29. The method of claim 25, wherein the human has an age of 13-18, 12-18, 10-18, 6-18, 10-13, 6-13, 2-13, 10-12, 6-12, 2-12, 6-10, 2-10, or 2-6 years, or any range therein.

30. The method of claim 25, wherein the subject is a child from 6 to 12 years old or having a body weight of less than 50 kg.

31. The method of claim 25, wherein the subject is a child of less than 2 years old.

32. The method of claim 6, wherein the primary hyperoxaluria is primary hyperoxaluria type 2 (PH2) or primary hyperoxaluria type 3 (PH3).

33. The method of any one of claims 1 to 5, wherein the hyperoxaluria is not caused by a primary hyperoxaluria.

34. The method of any one of claims 1 to 33, wherein the therapeutically effective amount reduces the uOxalate excretion to less than about 0.46 mmol/1.73 m² per 24 hours.

35. The method of any one of claims 1 to 34, wherein the therapeutically effective amount reduces the uOxalate excretion by at least about 50% relative to a uOxalate excretion prior to the treating.

36. The method of any one of claims 1 to 35, wherein the therapeutically effective amount brings a ratio of uOxalate to urine creatinine (uCreatinine) to a normal range of less than about 63.7 mg/mg, which is 80 mmol/mmol.

37. The method of any one of claims 1 to 36, wherein the therapeutically effective amount reduces a plasma glycolate (pGlycolate) concentration in an absolute amount and/or in a relative amount compared to a pGlycolate concentration prior to the treating.

38. The method of any one of claims 1 to 37, wherein the therapeutically effective amount increases an estimated glomerular filtration rate (eGFR) in an absolute amount and/or in a relative amount compared to an eGFR prior to the treating.

39. A method of treating kidney stones in a subject in need thereof, the method comprising administering to the subject a therapeutically effective amount of Compound 1, having the formula: 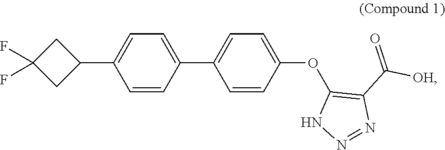 [SMILES: O=C(O)c1nn[nH]c1Oc1ccc(-c2ccc(C3CC(F)(F)C3)cc2)cc1]
a tautomer, a pharmaceutically acceptable salt, a hydrate, a solvate, or a combination thereof, wherein the therapeutically effective amount is a total daily dosage of about 20 milligrams (mg) to 3,500 mg.

40. The method of claim 39, wherein the therapeutically effective amount is a total daily dosage of about 20 mg to 2,500 mg.

41. The method of claim 39 or 40, wherein the treating reduces a size of the kidney stones.

42. A method of preventing the development, worsening, or recurrence of kidney stones in a subject in need thereof, the method comprising administering to the subject a therapeutically effective amount of Compound 1, having the formula: 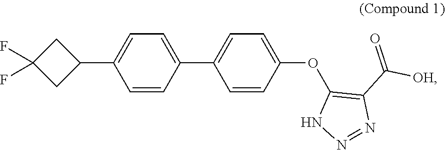 [SMILES: O=C(O)c1nn[nH]c1Oc1ccc(-c2ccc(C3CC(F)(F)C3)cc2)cc1]
a tautomer, a pharmaceutically acceptable salt, a hydrate, a solvate, or a combination thereof, wherein the therapeutically effective amount is a total daily dosage of about 20 milligrams (mg) to 3,500 mg.

43. The method of claim 42, wherein the therapeutically effective amount is a total daily dosage of about 20 mg to 2,500 mg.

44. The method of any one of claims 1 to 43, wherein Compound 1 is Compound 1a: 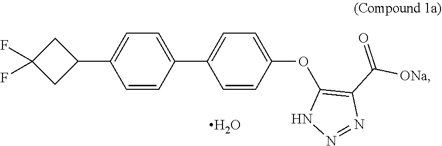
or a tautomer thereof.

45. The method of any one of claims 1 to 43, wherein the therapeutically effective amount is a total daily dosage of from about 20 mg to about 5,000 mg, from about 20 mg to about 4,500 mg, from about 20 mg to about 4,000 mg, from about 20 mg to about 3,500 mg, from about 20 mg to about 3,000 mg, from about 20 mg to about 2,500 mg, from about 20 mg to about 2,000 mg, from about 20 mg to about 1,500 mg, from about 20 mg to about 1,400 mg, from about 20 mg to about 1,300 mg, from about 20 mg to about 1,200 mg, from about 20 mg to about 1,100 mg, from about 20 mg to about 1,000 mg, from about 20 mg to about 900 mg, from about 20 mg to about 800 mg, from about 20 mg to about 700 mg, from about 20 mg to about 600 mg, from about 20 mg to about 550 mg, from about 20 mg to about 500 mg, from about 20 mg to about 450 mg, from about 20 mg to about 400 mg, from about 20 mg to about 350 mg, from about 20 mg to about 300 mg, from about 20 mg to about 250 mg, from about 20 mg to about 200 mg, from about 20 mg to about 150 mg, from about 20 mg to about 100 mg, 50 mg to about 5,000 mg, from about 50 mg to about 4,500 mg, from about 50 mg to about 4,000 mg, from about 50 mg to about 3,500 mg, from about 50 mg to about 3,000 mg, from about 50 mg to about 2,500 mg, from about 50 mg to about 2,000 mg, from about 50 mg to about 1,500 mg, from about 50 mg to about 1,400 mg, from about 50 mg to about 1,300 mg, from about 50 mg to about 1,200 mg, from about 50 mg to about 1,100 mg, from about 50 mg to about 1,000 mg, from about 50 mg to about 900 mg, from about 50 mg to about 800 mg, from about 50 mg to about 700 mg, from about 50 mg to about 600 mg, from about 50 mg to about 550 mg, from about 50 mg to about 500 mg, from about 50 mg to about 450 mg, from about 50 mg to about 400 mg, from about 50 mg to about 350 mg, from about 50 mg to about 300 mg, from about 50 mg to about 250 mg, from about 50 mg to about 200 mg, from about 50 mg to about 150 mg, from about 50 mg to about 100 mg, from about 75 mg to about 5,000 mg, from about 75 mg to about 4,500 mg, from about 75 mg to about 4,000 mg, from about 75 mg to about 3,500 mg, from about 75 mg to about 3,000 mg, from about 75 mg to about 2,500 mg, from about 75 mg to about 2,000 mg, from about 75 mg to about 1,500 mg, from about 75 mg to about 1,400 mg, from about 75 mg to about 1,300 mg, from about 75 mg to about 1,200 mg, from about 75 mg to about 1,100 mg, from about 75 mg to about 1,000 mg, from about 75 mg to about 900 mg, from about 75 mg to about 800 mg, from about 75 mg to about 700 mg, from about 75 mg to about 600 mg, from about 75 mg to about 550 mg, from about 75 mg to about 500 mg, from about 75 mg to about 450 mg, from about 75 mg to about 400 mg, from about 75 mg to about 350 mg, from about 75 mg to about 300 mg, from about 75 mg to about 250 mg, from about 75 mg to about 200 mg, from about 75 mg to about 150 mg, from about 75 mg to about 100 mg, from about 300 mg to about 5,000 mg, from about 300 mg to about 4,500 mg, from about 300 mg to about 4,000 mg, from about 300 mg to about 3,500 mg, from about 300 mg to about 3,000 mg, from about 300 mg to about 2,500 mg, from about 300 mg to about 2,000 mg, from about 300 mg to about 1,500 mg, from about 300 mg to about 1,400 mg, from about 300 mg to about 1,300 mg, from about 300 mg to about 1,200 mg, from about 300 mg to about 1,100 mg, from about 300 mg to about 1,000 mg, from about 300 mg to about 900 mg, from about 300 mg to about 800 mg, from about 300 mg to about 700 mg, from about 300 mg to about 600 mg, from about 300 mg to about 550 mg, from about 300 mg to about 500 mg, from about 300 mg to about 450 mg, from about 300 mg to about 400 mg, or from about 300 mg to about 350 mg of Compound 1, a tautomer, a pharmaceutically acceptable salt, a hydrate, a solvate, or a combination thereof.

46. The method of any one of claims 1 to 44, wherein the therapeutically effective amount is a total daily dosage of from about 20 mg to about 5,000 mg, from about 20 mg to about 4,500 mg, from about 20 mg to about 4,000 mg, from about 20 mg to about 3,500 mg, from about 20 mg to about 3,000 mg, from about 20 mg to about 2,500 mg, from about 20 mg to about 2,000 mg, from about 20 mg to about 1,500 mg, from about 20 mg to about 1,400 mg, from about 20 mg to about 1,300 mg, from about 20 mg to about 1,200 mg, from about 20 mg to about 1,100 mg, from about 20 mg to about 1,000 mg, from about 20 mg to about 900 mg, from about 20 mg to about 800 mg, from about 20 mg to about 700 mg, from about 20 mg to about 600 mg, from about 20 mg to about 550 mg, from about 20 mg to about 500 mg, from about 20 mg to about 450 mg, from about 20 mg to about 400 mg, from about 20 mg to about 350 mg, from about 20 mg to about 300 mg, from about 20 mg to about 250 mg, from about 20 mg to about 200 mg, from about 20 mg to about 150 mg, from about 20 mg to about 100 mg, 50 mg to about 5,000 mg, from about 50 mg to about 4,500 mg, from about 50 mg to about 4,000 mg, from about 50 mg to about 3,500 mg, from about 50 mg to about 3,000 mg, from about 50 mg to about 2,500 mg, from about 50 mg to about 2,000 mg, from about 50 mg to about 1,500 mg, from about 50 mg to about 1,400 mg, from about 50 mg to about 1,300 mg, from about 50 mg to about 1,200 mg, from about 50 mg to about 1,100 mg, from about 50 mg to about 1,000 mg, from about 50 mg to about 900 mg, from about 50 mg to about 800 mg, from about 50 mg to about 700 mg, from about 50 mg to about 600 mg, from about 50 mg to about 550 mg, from about 50 mg to about 500 mg, from about 50 mg to about 450 mg, from about 50 mg to about 400 mg, from about 50 mg to about 350 mg, from about 50 mg to about 300 mg, from about 50 mg to about 250 mg, from about 50 mg to about 200 mg, from about 50 mg to about 150 mg, from about 50 mg to about 100 mg, from about 75 mg to about 5,000 mg, from about 75 mg to about 4,500 mg, from about 75 mg to about 4,000 mg, from about 75 mg to about 3,500 mg, from about 75 mg to about 3,000 mg, from about 75 mg to about 2,500 mg, from about 75 mg to about 2,000 mg, from about 75 mg to about 1,500 mg, from about 75 mg to about 1,400 mg, from about 75 mg to about 1,300 mg, from about 75 mg to about 1,200 mg, from about 75 mg to about 1,100 mg, from about 75 mg to about 1,000 mg, from about 75 mg to about 900 mg, from about 75 mg to about 800 mg, from about 75 mg to about 700 mg, from about 75 mg to about 600 mg, from about 75 mg to about 550 mg, from about 75 mg to about 500 mg, from about 75 mg to about 450 mg, from about 75 mg to about 400 mg, from about 75 mg to about 350 mg, from about 75 mg to about 300 mg, from about 75 mg to about 250 mg, from about 75 mg to about 200 mg, from about 75 mg to about 150 mg, from about 75 mg to about 100 mg, from about 300 mg to about 5,000 mg, from about 300 mg to about 4,500 mg, from about 300 mg to about 4,000 mg, from about 300 mg to about 3,500 mg, from about 300 mg to about 3,000 mg, from about 300 mg to about 2,500 mg, from about 300 mg to about 2,000 mg, from about 300 mg to about 1,500 mg, from about 300 mg to about 1,400 mg, from about 300 mg to about 1,300 mg, from about 300 mg to about 1,200 mg, from about 300 mg to about 1,100 mg, from about 300 mg to about 1,000 mg, from about 300 mg to about 900 mg, from about 300 mg to about 800 mg, from about 300 mg to about 700 mg, from about 300 mg to about 600 mg, from about 300 mg to about 550 mg, from about 300 mg to about 500 mg, from about 300 mg to about 450 mg, from about 300 mg to about 400 mg, or from about 300 mg to about 350 mg of Compound 1a or a tautomer thereof.

47. The method of any one of claims 1 to 30, 32 to 44, and 46, wherein the therapeutically effective amount is a total daily dosage of from about 50 mg to about 1000 mg, from about 75 mg to about 1000 mg, from about 75 mg to about 800 mg, or from about 75 mg to about 600 mg of Compound 1a, when the subject is an adult of at least 18 years old or an adolescent of at least 12 years old with a body weight of at least 50 kg.

48. The method of claim 46 or 47, wherein the therapeutically effective amount is a total daily dosage of about 75 mg, about 300 mg, or about 600 mg of Compound 1a.

49. The method of claim 48, wherein the therapeutically effective amount is a total daily dosage of about 75 mg of Compound 1a.

50. The method of claim 48, wherein the therapeutically effective amount is a total daily dosage of about 300 mg of Compound 1a.

51. The method of claim 48, wherein the therapeutically effective amount is a total daily dosage of about 600 mg of Compound 1a.

52. The method of any one of claims 1 to 46, wherein, when the subject is a child from 6 to 12 years old or having a body weight of less than 50 kg, the therapeutically effective amount is adjusted to about ½, about ⅓, about ¼, or about ⅕ of a total daily dosage for the subject being an adult of at least 18 years old or an adolescent of at least 12 years old with a body weight of at least 50 kg.

53. The method of any one of claims 1 to 52, wherein Compound 1 or 1a is administered orally.

54. The method of any one of claims 1 to 53, wherein Compound 1 or 1a is administered once, twice, three times, or four times daily.

55. The method of claim 54, wherein Compound 1 or 1a is administered once daily.

56. The compounds of any one of claims 1 to 55, wherein Compound 1 or 1a is administered chronically.

57. The compounds of any one of claims 1 to 55, wherein Compound 1 or 1a is administered for at least 28 days.

58. The compounds of any one of claims 1 to 55, wherein Compound 1 or 1a is administered for at least 12 months.

59. The compounds of any one of claims 1 to 55, wherein Compound 1 or 1a is administered for at least 36 months.
